(19) United States
(12) Patent Application Publication
(10) Pub. No.: US 2023/0043128 A1
(43) Pub. Date: Feb. 9, 2023

(54) MULTIVALENT INFLUENZA VACCINES

(71) Applicant: SANOFI, Paris (FR)

(72) Inventors: Tim ALEFANTIS, Cambridge, MA (US); Sudha CHIVUKULA, Cambridge, MA (US)

(21) Appl. No.: 17/843,445

(22) Filed: Jun. 17, 2022

Related U.S. Application Data
(60) Provisional application No. 63/276,243, filed on Nov. 5, 2021.
(60) Provisional application No. 63/212,523, filed on Jun. 18, 2021.

(30) Foreign Application Priority Data
Oct. 13, 2021 (EP) 21315198.8
Nov. 5, 2021 (US) PCT/US2021/058250

(51) Int. Cl. A61K 39/145; A61K 31/7115; A61K 47/54; A61K 47/69

(52) U.S. Cl. CPC A61K 39/145; A61K 31/7115; A61K 47/543; A61K 47/6929; A61K 2039/51

(57) ABSTRACT

Provided are octavalent influenza vaccine compositions comprising eight mRNA, each mRNA comprising an open reading frame encoding a different influenza antigen. Also provided are lipid nanoparticles (LNPs) for delivering said mRNA.

### RELATED APPLICATIONS

This application claims the benefit of U.S. Provisional Application No. 63/212,523, filed on Jun. 18, 2021; U.S. Provisional Application No. 63/276,243, filed Nov. 5, 2021; PCT International Application No. PCT/US2021/058250, filed Nov. 5, 2021; and European Patent Application No. 21315198.8, filed Oct. 13, 2021; which are incorporated by reference in their entirety for all purposes.

### BACKGROUND OF THE INVENTION

Messenger RNA (mRNA) based vaccines provide a promising alternative to traditional subunit vaccines, which contain antigenic proteins derived from a pathogen. Antigen proteins are usually recombinantly made and require bacterial fermentation and/or cell culture, as well as complex purification. Vaccines based on mRNA allow de novo expression of complex antigens in the vaccinated subject, which in turn allows proper post-translational modification and presentation of the antigens in its natural conformation. Unlike traditional technologies, the manufacture of mRNA vaccines does not require complex and costly bacterial fermentation, tissue culture, and purification processes. Moreover, once established, the manufacturing process for mRNA vaccines can be used for a variety of antigens, enabling rapid development and deployment of mRNA vaccines. Further, mRNA vaccines are inherently safe delivery vectors as they express the antigens only transiently and do not integrate into the host genome. Because antigens encoded by mRNAs are produced in vivo in the vaccinated individual, mRNA vaccines are especially effective in eliciting both humoral and T cell mediated immunity.

Current approved influenza vaccines are either live attenuated influenza vaccines or inactivated influenza vaccines, which are often produced in cell culture or eggs. Moreover, multiple strains of influenza may circulate within populations each year, making it difficult for a single influenza vaccine to offer robust protection against multiple strains. Accordingly, there exists a need for mRNA-based influenza vaccines, including multivalent mRNA-based influenza vaccines that target multiple influenza strains.

### SUMMARY OF THE INVENTION

The present disclosure provides an influenza vaccine composition, comprising eight messenger RNA (mRNA), each mRNA comprising an open reading frame (ORF) encoding a different influenza antigen.

In certain embodiments, the composition comprises eight mRNA encoding (i) one or more hemagglutinin (HA) antigens, (ii) one or more neuraminidase (NA) antigens, or (iii) at least one HA antigen and at least one NA antigen.

In certain embodiments, the composition comprises one or more mRNA encoding antigens of influenza A, B and/or C viruses.

In certain embodiments, the antigens are HA and/or NA antigens of influenza A and influenza B viruses.

In certain embodiments, the NA antigens of influenza A viruses are selected from subtypes N1, N2, N3, N4, N5, N6, N7, N8, N9, N10, and N11.

In certain embodiments, the HA and NA antigens of influenza B viruses are from the influenza B/Yamagata lineage or the influenza B/Victoria lineage.

In certain embodiments, the HA antigen and NA antigen is selected from the group consisting of H1N1, H3N2, H2N2, H5N1, H7N9, H7N7, H1N2, H9N2, H7N2, H7N3, H5N2, and H10N7 subtypes and/or B/Yamagata and B/Victoria lineages.

In certain embodiments, the composition comprises one mRNA encoding an H3 HA antigen, one mRNA encoding an H1 HA antigen, one mRNA encoding an HA antigen from the Influenza B/Yamagata lineage, and one mRNA encoding an HA antigen from the Influenza B/Victoria lineage.

In certain embodiments, the composition comprises one mRNA encoding an H3 HA antigen, one mRNA encoding an N2 NA antigen, one mRNA encoding an H1 HA antigen, one mRNA encoding an N1 NA antigen, one mRNA encoding an HA antigen from the Influenza B/Yamagata lineage, one mRNA encoding an NA antigen from the Influenza B/Yamagata lineage, one mRNA encoding an HA antigen from the Influenza B/Victoria lineage, and one mRNA encoding an NA antigen from the Influenza B/Victoria lineage.

In certain embodiments, the ORF is codon optimized.

In certain embodiments, the mRNA molecule comprises at least one 5′ untranslated region (5′ UTR), at least one 3′ untranslated region (3′ UTR), and at least one polyadenylation (poly(A)) sequence.

In certain embodiments, the mRNA comprises at least one chemical modification.

In certain embodiments, at least 20%, at least 30%, at least 40%, at least 50%, at least 60%, at least 70%, at least 80%, at least 85%, at least 90%, at least 95%, or 100% of the uracil nucleotides in the mRNA are chemically modified.

In certain embodiments, at least 20%, at least 30%, at least 40%, at least 50%, at least 60%, at least 70%, at least 80%, at least 85%, at least 90%, at least 95%, or 100% of the uracil nucleotides in the ORF are chemically modified.

In certain embodiments, the chemical modification is selected from the group consisting of pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine.

In certain embodiments, the chemical modification is selected from the group consisting of pseudouridine, N1-methylpseudouridine, 5-methylcytosine, 5-methoxyuridine, and a combination thereof.

In certain embodiments, the chemical modification is N1-methylpseudouridine.

In certain embodiments, the mRNA is formulated in a lipid nanoparticle (LNP).

In certain embodiments, the LNP comprises at least one cationic lipid.

In certain embodiments, the cationic lipid is biodegradable. In certain embodiments, the cationic lipid is not biodegradable.

In certain embodiments, the cationic lipid is cleavable. In certain embodiments, the cationic lipid is not cleavable.

In certain embodiments, the cationic lipid is selected from the group consisting of OF-02, cKK-E10, GL-HEPES-E3-E10-DS-3-E18-1, GL-HEPES-E3-E12-DS-4-E10, and GL-HEPES-E3-E12-DS-3-E14.

In certain embodiments, the LNP further comprises a polyethylene glycol (PEG) conjugated (PEGylated) lipid, a cholesterol-based lipid, and a helper lipid.

In certain embodiments, the LNP comprises:

a cationic lipid at a molar ratio of 35% to 55%;

a polyethylene glycol (PEG) conjugated (PEGylated) lipid at a molar ratio of 0.25% to 2.75%;

a cholesterol-based lipid at a molar ratio of 20% to 45%; and

a helper lipid at a molar ratio of 5% to 35%, wherein all of the molar ratios are relative to the total lipid content of the LNP.

In certain embodiments, the LNP comprises:

a cationic lipid at a molar ratio of 40%;

a PEGylated lipid at a molar ratio of 1.5%;

a cholesterol-based lipid at a molar ratio of 28.5%; and a helper lipid at a molar ratio of 30%.

In certain embodiments, the PEGylated lipid is dimyristoyl-PEG2000 (DMG-PEG2000) or 2-[(polyethylene glycol)-2000]-N,N-ditetradecylacetamide (ALC-0159).

In certain embodiments, the cholesterol-based lipid is cholesterol.

In certain embodiments, the helper lipid is 1,2-dioleoyl-SN-glycero-3-phosphoethanolamine (DOPE) or 1,2-distearoyl-sn-glycero-3-phosphocholine (DSPC).

In certain embodiments, the LNP comprises:

a cationic lipid is selected from the group consisting of OF-02, cKK-E10, GL-HEPES-E3-E10-DS-3-E18-1, GL-HEPES-E3-E12-DS-4-E10, and GL-HEPES-E3-E12-DS-3-E14, at a molar ratio of 40%;

DMG-PEG2000 at a molar ratio of 1.5%;

cholesterol at a molar ratio of 28.5%; and

DOPE at a molar ratio of 30%.

In certain embodiments, the LNP has an average diameter of 30 nm to 200 nm. In certain embodiments, the LNP has an average diameter of 80 nm to 150 nm.

In certain embodiments, the influenza vaccine composition comprises between 1 mg/mL to 10 mg/mL of the LNP.

In certain embodiments, the LNP comprises between 1 and 20 mRNA molecules. In certain embodiments, the LNP comprises 5-10 or 6-8 mRNA molecules.

In certain embodiments, the LNP comprises two or more mRNA, wherein each mRNA encodes a different influenza antigen.

In certain embodiments, the composition comprises eight LNPs, wherein each LNP comprises an mRNA encoding a different influenza antigen.

In certain embodiments, the composition is formulated for intramuscular injection.

In certain embodiments, the composition comprises a phosphate-buffer saline.

In one aspect, the disclosure provides a method of eliciting an immune response in a subject in need thereof, comprising administering to the subject, optionally intramuscularly, intranasally, intravenously, subcutaneously, or intradermally, a prophylactically effective amount of the influenza vaccine composition described above.

In one aspect, the disclosure provides a method of preventing influenza infections or reducing one or more symptoms of influenza infections, comprising administering to the subject, optionally intramuscularly, intranasally, intravenously, subcutaneously, or intradermally, a prophylactically effective amount of the influenza vaccine composition described above.

In certain embodiments, the influenza vaccine composition elicits an immune response against one or more seasonal and/or pandemic influenza strains.

In certain embodiments, the method comprises administering to the subject one or more doses of the influenza vaccine composition, each dose comprising about 1 μg to about 250 μg of mRNA.

In certain embodiments, the method comprises administering to the subject one or more doses of the influenza vaccine composition, each dose comprising about 2.5, 5, 15, 45, or 135 μg of mRNA.

In certain embodiments, the method comprises administering to the subject two doses of the influenza vaccine composition with an interval of 2-6, optionally 4, weeks.

In another aspect, the disclosure provides for the use of the influenza vaccine composition described above for the manufacture of a medicament for use in treating a subject in need thereof.

In certain embodiments, the influenza vaccine composition is for use in treating a subject in need thereof.

In another aspect, the disclosure provides a kit comprising a container comprising a single-use or multi-use dosage of the composition described above, optionally wherein the container is a vial or a pre-filled syringe or injector.

In certain embodiments, the influenza antigens comprise an influenza virus HA antigen and/or an influenza virus NA antigen having a molecular sequence identified or designed from a machine learning model.

### BRIEF DESCRIPTION OF THE DRAWINGS

FIG. 1A is a pair of graphs showing the expression of human erythropoietin (hEPO) in mice treated with various LNP formulations of hEPO mRNA. Panel a): LNP formulations “Lipid A” and “Lipid B” compared to MC3. Bars represent means and standard deviations. Panel b): Formulation made with cationic lipid OF-02. PEG: DMG-PEG2000. Cholest: cholesterol. “Lipid A”: LNP composition containing OF-02, DMG-PEG2000, cholesterol, and DOPE, in this order, at a molar ratio of 40:1.5:28.5:30, unless otherwise indicated. “Lipid B”: LNP composition containing cKK-E10, DMG-PEG2000, cholesterol, and DOPE, in this order, at a molar ratio of 40:1.5:28.5:30.

FIG. 1B is a pair of graphs showing expression of hEPO in mice and non-human primates (NHPs) using LNP formulations Lipid A and Lipid B.

FIGS. 2A and 2B are a pair of graphs showing that Lipid A and Lipid B LNP formulations with mRNA encoding hemagglutinin (HA) of strain A/California/7/2009 (H1N1) (CA09) induced robust functional antibodies (FIG. 2A) and protected mice against death or severe weight loss (more than 20%) when challenged with a pandemic strain of influenza virus (FIG. 2B). Hemagglutinin inhibition (HAI) titers are reported as log 10 for serum samples taken at study days 0, 14, 28, 42, 56, 92, and 107. Bars are geometric means and geometric standard deviations. Daily weights were measured after intranasal challenge (day 93) with 4LD₅₀ of A/Belgium/2009 (H1N1) (Belgium09). Weights are presented as the percentage of weight lost from the day of challenge. Euthanasia occurred for mice losing more than 20% of their starting body weight and for all mice 14 days post-infection (day 107). rHA: recombinant hemagglutinin. AF03: an oil-in-water emulsion adjuvant. Diluent=PBS. LLOQ=lower limit of quantitation. 1/40=1/40 minimum target, which refers to HAI antibody titers associated with 50% reduction in the risk of influenza infection or disease in healthy adults (Coudeville et al., BMC Med Res Methodol. (2010) 10:18). Dashed line in FIG. 2B=20% weight loss cut off with respect to weight on the day of challenge.

FIGS. 3A and 3B are a pair of graphs showing that A/Michigan/45/2015 (Mich15) neuraminidase (NA) mRNA formulated with Lipid A LNP induced robust functional antibodies (FIG. 3A) and protected mice against weight loss and death when challenged with a pandemic strain of influenza virus (FIG. 3B). Neuraminidase inhibition (NAI) titers are reported as log 10 for serum samples taken at study days 14, 28, 42, 56, 88, and 114. Daily weights were observed after intranasal challenge (day 89 for the one-dose groups or day 117 for the two-dose groups) with 4LD₅₀ of Belgium09. Weights are presented as the percentage of weight lost from the day of challenge. Euthanasia occurred for mice losing more than 20% of their starting body weight and for all mice 14 days post-infection (day 103 for the 1 dose groups or day 131 for the 2 dose groups). Bars are means and standard deviations. Upper dashed line in FIG. 3A=upper limit of quantitation. Lower dashed line in FIG. 3A=lower limit of quantitation. Dashed line in FIG. 3B=20% weight loss cut off with respect to weight on the day of challenge. mRNA dosed: 0.4 or 0.016 μg mRNA encoding Mich15 NA. Control: 0.6 μg mRNA encoding hEPO or diluent (PBS).

FIG. 4 is a graph showing that Lipid A and Lipid B LNP formulations with CA09 HA mRNA (10 μg) induced robust functional antibodies in cynomolgus macaque monkeys. HAI titers are reported as log 2 for serum samples taken at study days 0, 14, 28, 42, and 56.

FIGS. 5A-C show the MRT1400 mRNA encoding for influenza virus A/Singapore/INFIMH160019/2016 (Sing16; H3N2) HA hemagglutinin. FIG. 5A: an alignment of the wildtype (WT) gene and a codon-optimized gene (MRT10279) for the HA antigen. FIG. 5B: the structure of the mRNA. FIG. 5C: the sequence of the mRNA.

FIG. 6 is a pair of graphs showing that Lipid A and Lipid B LNP formulations with MRT1400 or NA mRNA induced robust functional antibodies in mice. First injection was given at study day 0 and second injection was given at study day 28. Left Panel: HAI titers are reported as log 10 for serum samples taken at study days 14, 28, 42, and 56. Right Panel: NAI titers are reported as log 10 for serum samples taken at study days 14, 28, 42, and 56. Bars are geometric means and geometric standard deviations. Dashed line=lower limit of quantitation.

FIG. 7A is a graph showing that Lipid A and Lipid B LNP formulations with MRT 1400 induced robust functional antibodies in NHPs. HAI titers are reported as log 2 for serum samples taken at study days 0, 14, 28, 42, and 56. First injection was given at study day 0 and second injection was given at study day 28. Bars are means and standard deviation. Upper dashed line=1/40 minimum target. Lower dashed line=lower limit of detection.

FIGS. 7B and 7C are a pair of graphs showing that a Lipid A LNP formulation (MRT5400) containing MRT1400 mRNA induced functional antibodies (FIG. 7B) and robust ELISA titers (FIG. 7C) in cynomolgus macaque monkeys at four dose levels: 15, 45, 135 and 250 μg of mRNA. HAI and ELISA titers are reported as log 2 for serum samples taken at study days 0, 14, 28, 42, and 56. First injection was given at study day 0 and second injection given at study day 28. Bars are means and standard deviations. Dash line=1/40 minimum target.

FIGS. 8A and 8B are panels of graphs showing the T cell cytokine response of cynomolgus macaques after a second vaccination with Lipid A LNP formulation MRT5400 in three dose level groups (250 μg, 135 μg, and 45 μg of mRNA). IFN-γ and IL-13 induced by re-stimulation with either the recombinant HA (rHA) protein (left panel) or the pooled peptides (right panel) were assessed in peripheral blood mononuclear cells (PMBC) on day 42 by ELISPOT assays. The frequencies of PBMC secreting IFN-γ (FIG. 8A) or IL-13 (FIG. 8B) were calculated as spots forming cells (SFC) per million PBMC. Each symbol represents an individual sample, and the bar represents the standard deviation.

FIG. 9A is a pair of graphs showing that Lipid A LNP formulations containing modified and unmodified CA09 HA mRNA were comparable as indicated by HAI titers in vaccinated mice. HAI titers are reported as log 2 for serum samples taken at study days 14, 28, 42, and 56. First injection was given at study day 0 and second injection was given at study day 28. Bars are means and standard deviation. Upper dashed line=1/40 minimum target. Lower dashed line=lower limit of quantitation.

FIG. 9B is a pair of graphs showing that Lipid A LNP formulations containing modified and unmodified CA09 HA mRNA were comparable as indicated by ELISA titers in mice. Total IgG ELISA titers are reported as log 10 for serum samples taken at study days 14, 28, 42, and 56. First injection was given at study day 0 and second injection was given at study day 28. Dashed line=lower limit of quantitation.

FIGS. 10A and 10B are a pair of graphs showing that bivalent Lipid A LNP formulations with CA09 HA mRNA and Sing16 HA mRNA induced robust functional antibodies as assessed by HAI titers (CA09 (FIG. 10A) and Sing16 (FIG. 10B)) in Balb/c mice at a dose of 0.4 μg of total mRNA. 0.4 μg mRNA was dosed as a co-encapsulated mRNA-LNP formulation, or each HA mRNA was separately administered with 0.2 μg going into each leg. Each HA mRNA was also co-encapsulated into a formulation with non-coding mRNA to control for total mRNA packing into the LNP. The diluent group received mRNA-LNP diluent buffer. HAI titers are reported for serum samples taken at study days −2 (baseline), 14, 28, and 42. FIG. 10B only shows study days −2 (baseline from pooled sera) and 42. First injection was given at study day 0 and second injection given at study day 28. Bars are geometric means and geometric standard deviations. Dashed line=lower limit of quantitation.

FIG. 11 shows the functional verification of mRNA-LNP Formulations. Panel (a) is a graph showing the expression of firefly (FF) luciferase in BALB/c mice: a single dose of Luciferase FF mRNA-LNP (5, 1, 0.1, 0.05 μg) was injected in mice (n=4) by IM route. Luciferin (3 mg) was injected at the time of whole animal imaging, using IVIS Spectrum, Perkin Elmer recording bioluminescence intensity. Images of whole animal average radiance at 6, 24, 48 and 72 h after injection were taken. Radiance recorded for 1, 0.5, 0.1 and 0.05 μg dose administrations of Luc mRNA-LNP are shown in the graph. Panel (b) shows whole animal images indicating total flux of luminescence, at 6 to 72 hours. Total flux of luminescence in groups of mice (n=4) receiving 0.1 μg dose of FF-LNP are shown. Panel (c) shows the expression of hEPO in BALB/c mice. A single dose of hEPO mRNA-LNP (0.1 μg) was injected in BALB/c mice by IM route. hEPO expression was quantified in serum at 6 hours and 24 hours after administration using ELISA. Bars represent means and standard deviations. Panel (d) shows the expression of hEPO in NHP. A single dose of hEPO mRNA-LNP (10 μg) was injected in Cynomolgus macaques by IM route. hEPO expression was quantified in serum at 6, 24, 48, 72, and 96 hours after administration, using ELISA. Bars represent means and standard deviations.

FIG. 12 shows the serological evaluation of HA mRNA-LNP vaccine in mice. BALB/c mice (n=8 per group) were immunized twice IM, 4 weeks apart with 2, 0.4, 0.08, and 0.016 μg of either Cal09 HA mRNA-LNP or Sing16 HA mRNA-LNP. ELISA titers recorded for sera collected at days 14, 28, 42, 56 against CA09 (Cal09) H1N1 influenza virus recombinant HA (left panel) and Sing16 H3N2 influenza virus recombinant HA (right panel) are shown.

FIG. 13 shows the serological evaluation of HA mRNA-LNP vaccine in mice. BALB/c mice (n=8 per group) were immunized twice IM, 4 weeks apart with 2, 0.4, 0.08 and 0.016 μg of either CA09 HA mRNA-LNP or Sing16 HA mRNA-LNP. Log₁₀ HAI titers recorded against CA09 H1N1 influenza virus (left panel) and Sing16 H3N2 influenza virus (right panel) are shown.

FIG. 14 shows the serological evaluation of NA mRNA-LNP vaccine in mice. BALB/c mice (n=8 per group) were immunized twice IM 4 weeks apart with 2, 0.4, 0.08, and 0.016 μg of either Mich15 NA mRNA-LNP or Sing16 NA mRNA-LNP. Total IgG titers recorded for sera collected at days 0, 14, 28, 42, 56 against Mich15 N1 influenza virus recombinant NA (left panel) and Sing16 N2 virus recombinant NA (right panel) are shown.

FIG. 15 shows the serological evaluation of NA mRNA-LNP vaccine in mice. BALB/c mice (n=8 per group) were immunized twice IM 4 weeks apart with 2, 0.4, 0.08 and 0.016 μg of either Mich15 NA mRNA-LNP or Sing16 NA mRNA-LNP. Log₁₀ NAI (ELLA) titers recorded for sera against Mich2015 (N1): A/Mallard/Sweden/2002 (H6) chimeric influenza virus (left panel) and Sing16 (N2): A/Mallard/Sweden/2002 (H6) chimeric virus (right panel) are shown.

FIGS. 16A and 16B show the protective efficacy of CA09 HA mRNA-LNP vaccine in mice after lethal A/Belgium/2009 H1N1 virus challenge. Mice (n=8) received two IM doses of CA09 HA mRNA-LNP (0.4 μg each) on day 0 and day 28. Control animals received two IM doses of diluent on day 0 and day 28. FIG. 16A shows the HAI titers reported as Log₁₀ for serum samples taken at study days 0, 14, 28, 42, 56, 92, and 107. FIG. 16B shows daily weights after intranasal challenge on day 93 with 4LD₅₀ of A/Belgium/2009 H1N1 strain. Weights are presented as the percentage of weight lost from the day of challenge. Individual lines represent each animal.

FIGS. 17A-B show the protective efficacy of a single dose of unmodified Mich15 NA mRNA-LNP in mice after lethal A/Belgium/2009 H1N1 virus challenge. Mice (n=16) were injected by the IM route with 0.4 μg or 0.016 μg of Mich15 NA mRNA-LNP. Half of the mice only received one injection (1 dose) on study day 0, while the other half (2 doses) received two injections given at study day 0 and day 28. Control animals received two IM doses of hEPO mRNA-LNP (0.6 μg) on day 0 and day 28. FIG. 17A shows the NAI titers are reported as Log₁₀ for serum samples taken at study days 0, 14, 28, 42, 56, 88, and 114. FIG. 17B shows the daily weight change after intranasal challenge on day 89 for single dose group and day 117 (89 days after second dose) for two dose group with 4LD50 of Belgium09 H1N1. Weights are presented as the percentage of weight lost from the day of challenge. Individual lines represent each animal.

FIG. 18 shows the serological evaluation of HA Sing16 HA mRNA-LNP vaccine in NHP. Cynomolgus macaques (n=6 per group) were injected twice, 4 weeks apart by IM route, with 15, 45 or 135 μg of Sing16 HA mRNA-LNP. Serum samples were collected at days −6, 14, 28, 42, and 56. Log₁₀ IgG titers against recombinant HA protein of Sing16 virus are shown.

FIGS. 19A and 19B show the serological evaluation of HA Sing16 HA mRNA-LNP vaccine in NHP. Cynomolgus macaques (n=6 per group) were injected twice, 4 weeks apart by IM route, with 15, 45 or 135 μg of Sing16 HA mRNA-LNP. Serum samples were collected at days 0, 14, 28, 42, and 56. Log₁₀ HAI titers (FIG. 19A) and Log₁₀ micro-neutralization (MN) titers (FIG. 19B) against Sing2016 virus are shown.

FIGS. 20A and 20B show T cell responses in NHP vaccinated with Sing16 HA mRNA-LNP vaccine. Cynomolgus macaques (n=6 per group) were injected twice, 4 weeks apart by IM route, with 45, 135, or 250 μg of Sing16 HA mRNA-LNP. T cells were determined by ELISPOT on day 42 in PBMC stimulated in vitro with peptide pools to represent the entire HA open reading frame. The responses of PBMC secreting IFN-γ (FIG. 20A) or IL-13 (FIG. 20B) calculated as spots forming cells (SFC) per million PBMC are shown. Each symbol represents an individual sample, and the bar represent the geometric mean for the group.

FIG. 21 shows the secretion of Sing16 H3-specific IgG by memory B cells on day 180 in NHP vaccinated with Sing16 HA mRNA-LNP vaccine. Cynomolgus macaques (n=6 per group) were injected twice, 4 weeks apart by IM route, with 15 or 45 μg of Sing16 HA mRNA-LNP. The Human IgG single-color memory B cell ELISPOT kit (CAT #NC1911372, CTL) was used to measure Sing16/H3-specific and total IgG⁺ antibody-secreting cells (ASCs). Differentiation of MBCs into ASCs was performed in PBMC collected at day 180 by using a stimulation cocktail provided by the kit. The number of IgG⁺ and number of Sing16/H3-specific ASCs was calculated per million of PBMCs for each animal and the frequency of antigen-specific ASCs is shown.

FIG. 22 shows the delivery of bivalent combinations of influenza vaccine in mice. BALB/c mice (n=8 per group) were immunized twice IM, 4 weeks apart with a total 0.4 μg of bivalent combinations co-encapsulated mRNA transcripts (1:1 wt/wt, half volume per each leg) or 0.2 μg each monovalent which was separately formulated and immunized different legs. H1H3 combo constituting CA09 HA mRNA-LNP, Sing16 HA mRNA-LNP; H3N2 combo of Sing16 HA mRNA-LNP and Sing16 NA mRNA-LNP and N1N2 combo of Mich15 NA mRNA-LNP and Perth09 NA mRNA-LNP were tested in sera collected a day 0, 14, 28, 42, against corresponding virus. Panel (a) shows HAI titers recorded against CA09 H1N1 influenza virus and Sing2016 H3N2. Panel (b) shows the HAI and NAI titers recorded against Sing2016 H3N2 and A/Mallard/Sweden/2002 (H6) chimeric influenza virus and H6N2 A/Perth/09 virus F1919D (N2) virus, respectfully. Panel (c) shows NAI titers recorded against Mich15 (N1): A/Mallard/Sweden/2002 (H6) chimeric influenza virus and H6N2 A/Perth/09 virus F1919D (N2) virus.

FIG. 23 shows the delivery of quadrivalent combinations of influenza vaccines in NHP. Cynomolgus macaques (n=6 per group) were immunized twice IM, 4 weeks apart with a total 10 μg of quadrivalent combinations of co-encapsulated mRNA transcripts (1:1:1:1 wt/wt). H2H3N1N2 combo consisting of CA09 HA mRNA, Sing16 HA mRNA, Mich15 NA mRNA, and Perth09 NA mRNA. HIH3 combo constituting CA09 HA mRNA, Sing16 HA mRNA and 2× non-coding mRNA (ncmRNA); H3N2 combo of Sing16 HA mRNA and Perth09 NA mRNA and 2× non-coding mRNA. N1N2 combo of Mich15 NA mRNA, Perth09 NA mRNA-LNP, and 2× non-coding mRNA. H1 consisting of CA09 HA mRNA and 3× non-coding mRNA. H3 consisting of Sing16 HA mRNA and 3× non-coding mRNA. N1 consisting of Mich15 NA mRNA and 3× non-coding mRNA. N2 consisting of Perth09 NA mRNA and 3× non-coding mRNA. Inhibitory titers were tested in sera collected a day 0, 14, 28, 42, against corresponding virus. Panel (a) shows the HAI titers recorded against CA09 H1N1 influenza virus and Sing16 H3N2. Panel (b) shows the NAI titers recorded against Mich15 (N1): A/Mallard/Sweden/2002 (H6) chimeric influenza virus and H6N2 Perth/09 virus F1919D (N2) virus.

FIG. 24 depicts a graph showing the expression of human erythropoietin (hEPO) in mice treated with various LNP formulations of hEPO mRNA. LNP formulations “Lipid A,” “Lipid B,” “Lipid C,” “Lipid D,” and “Lipid E” are shown. Bars represent means and standard deviations. The LNP compositions contain the cationic lipid, DMG-PEG2000, cholesterol, and DOPE, in this order, at a molar ratio of 40:1.5:28.5:30.

FIG. 25 depicts a graph showing the expression of hEPO in non-human primates (NHPs) treated with various LNP formulations of hEPO mRNA. LNP formulations “Lipid A,” “Lipid B,” “Lipid C,” “Lipid D,” and “Lipid E” are shown. Bars represent means and standard deviations. The LNP compositions contain the cationic lipid, DMG-PEG2000, cholesterol, and DOPE, in this order, at a molar ratio of 40:1.5:28.5:30.

FIG. 26 depicts a graph showing HAI titers at day 28 and day 42 post injection with various LNP formulations of HA mRNA. LNP formulations “Lipid A,” “Lipid B,” “Lipid C,” “Lipid D,” and “Lipid E” are shown. Bars represent means and standard deviations. The LNP compositions contain the cationic lipid, DMG-PEG2000, cholesterol, and DOPE, in this order, at a molar ratio of 40:1.5:28.5:30.

FIG. 27 depicts a graph showing Cal09 H1 HAI titers at day 28 and day 42 post injection with various LNP formulations of HA mRNA. LNP formulations “Lipid A,” “Lipid B,” “Lipid C,” “Lipid D,” and “Lipid E” are shown. Bars represent means and standard deviations. The LNP compositions contain the cationic lipid, DMG-PEG2000, cholesterol, and DOPE, in this order, at a molar ratio of 40:1.5:28.5:30.

FIG. 28 depicts a graph showing Sing16 H3 HAI titers at day 28 and day 42 post injection with various LNP formulations of HA mRNA. LNP formulations “Lipid A,” “Lipid B,” “Lipid C,” “Lipid D,” and “Lipid E” are shown. Bars represent means and standard deviations. The LNP compositions contain the cationic lipid, DMG-PEG2000, cholesterol, and DOPE, in this order, at a molar ratio of 40:1.5:28.5:30.

FIG. 29 depicts HAI titers for quadrivalent and octavalent mRNA-LNP vaccines administered to mice for 4 different influenza strains.

FIG. 30 depicts HINT values for quadrivalent and octavalent mRNA-LNP vaccines, administered to ferrets for 4 different influenza strains.

FIG. 31 depicts NAI titers for quadrivalent and octavalent mRNA-LNP vaccines, administered to mice for 4 different influenza strains.

FIG. 32 depicts NAI titers for quadrivalent and octavalent mRNA-LNP vaccines, administered to ferrets for 4 different influenza strains. Samples were obtained on day 20 (D20) after the second dose of vaccine.

FIG. 33 depicts NAI titers for quadrivalent and octavalent mRNA-LNP vaccines, administered to ferrets for 4 different influenza strains. Samples were obtained on day 42 (D42) after the second dose of vaccine.

### DETAILED DESCRIPTION OF THE INVENTION

The present disclosure provides novel lipid nanoparticle (LNP) formulations for delivering mRNA vaccines in vivo and methods of making the vaccines. The LNPs are made of a mixture of four lipids: a cationic lipid, a polyethylene glycol (PEG)-conjugated lipid, a cholesterol-based lipid, and a helper lipid. The LNPs encapsulate mRNA molecules. The encapsulated mRNA molecules can be comprised of naturally-occurring ribonucleotides, chemically modified nucleotides, or a combination thereof, and can each or collectively code for one or more proteins.

The inventors have discovered the present formulations through screening combinatorial libraries of lipid components. The present LNPs encapsulate and protect the mRNA payload from degradation and facilitate cellular uptake of the encapsulated mRNA. The LNPs described herein have enhanced transfection efficiency, promote endosomal escape of the mRNA, and consequently have improved potency as demonstrated by enhanced expression in vivo and in vitro when compared to industrial formulations described in literature. For example, the LNPs disclosed herein have superior stability and/or potency profiles compared to known LNPs, e.g., heptatriaconta-6,9,28,31-tetraen-19-yl 4-(dimethylamino)butanoate (aka. DLin-MC3-DMA or MC3; Semple et al., Nat Biotechnol. (2010) 28:172-6) or di((Z)-non-2-en-1-yl) 9-((4-(dimethylamino)butanoyl)oxy) heptadecanedioate (aka. L319; Maier et al., Mol Ther. (2013) 21(8):1570-8). As further described below, the present formulations encapsulating an mRNA encoding hEPO, when delivered in vivo, led to high levels of erythropoietin circulating in blood at 6 hours and 24 hours, with an up to 12-fold increase, relative to the industrial standard, the MC3 LNP formulation. Similarly, high potency has been found with other mRNAs, such as those encoding influenza antigens, in both murine and non-human primate models.

The mRNA vaccines as formulated herein can be used to induce a balanced immune response comprising both cellular and humoral immunity. Because the advantages of the present LNP formulations are not sequence-specific, these formulations can be used to deliver mRNAs that encode a variety of antigens, allowing rapid deployment in epidemic or pandemic situations. Further, the present LNP-formulated mRNA vaccines are highly immunogenic and therefore provide significant dose sparing possibility.

### I. Lipid Nanoparticle (LNP)

The LNPs of the disclosure comprise four categories of lipids: (i) an ionizable lipid (e.g., a cationic lipid); (ii) a PEGylated lipid; (iii) a cholesterol-based lipid, and (iv) a helper lipid.

A. Ionizable Lipids

An ionizable lipid facilitates mRNA encapsulation and may be a cationic lipid. A cationic lipid affords a positively charged environment at low pH to facilitate efficient encapsulation of the negatively charged mRNA drug substance.

In some embodiments, the cationic lipid is OF-02:

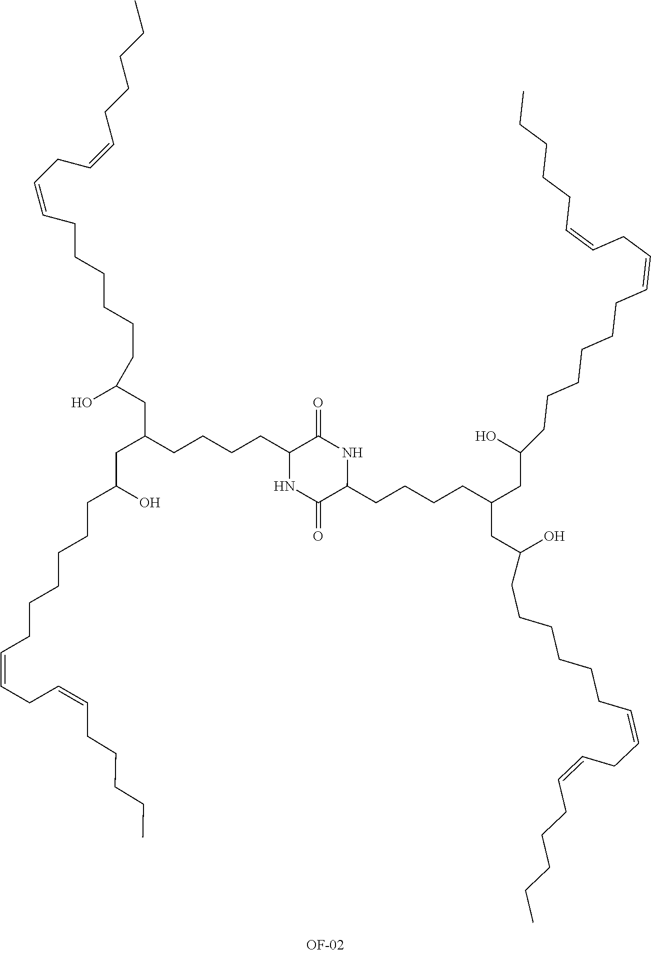 [SMILES: CCCCC/C=C\C/C=C\CCCCCCC(O)CC(CCCCC1NC(=O)C(CCCCC(CC(O)CCCCCC/C=C\C/C=C\CCCCC)CC(O)CCCCCC/C=C\C/C=C\CCCCC)NC1=O)CC(O)CCCCCC/C=C\C/C=C\CCCCC]

OF-02 is a non-degradable structural analog of OF-Deg-Lin. OF-Deg-Lin contains degradable ester linkages to attach the diketopiperazine core and the doubly-unsaturated tails, whereas OF-02 contains non-degradable 1,2-amino-alcohol linkages to attach the same diketopiperazine core and the doubly-unsaturated tails (Fenton et al., Adv Mater. (2016) 28:2939; U.S. Pat. No. 10,201,618). An exemplary LNP formulation herein, Lipid A, contains OF-2.

In some embodiments, the cationic lipid is cKK-E10 (Dong et al., PNAS (2014) 111(11):3955-60; U.S. Pat. No. 9,512,073):

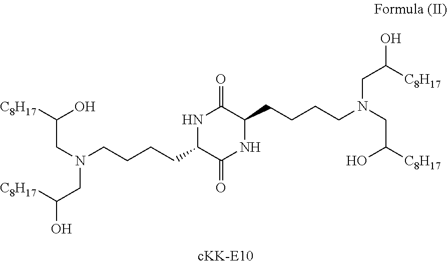

An exemplary LNP formulation herein, Lipid B, contains cKK-E10.

In some embodiments, the cationic lipid is GL-HEPES-E3-E10-DS-3-E18-1 (2-(4-(2-((3-(Bis((Z)-2-hydroxyoctadec-9-en-1-yl)amino)propyl)disulfaneyl)ethyl)piperazin-1-yl)ethyl 4-(bis(2-hydroxydecyl)amino)butanoate), which is a HEPES-based disulfide cationic lipid with a piperazine core, having the Formula III:

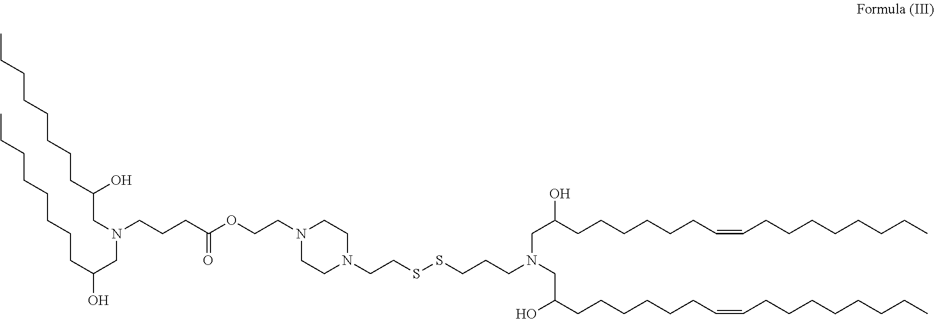 [SMILES: CCCCCCCC/C=C\CCCCCCC(O)CN(CCCSSCCN1CCN(CCOC(=O)CCCN(CC(O)CCCCCCCC)CC(O)CCCCCCCC)CC1)CC(O)CCCCCC/C=C\CCCCCCCC]

An exemplary LNP formulation herein, Lipid C, contains GL-HEPES-E3-E10-DS-3-E18-1. Lipid C has the same composition as Lipid A or Lipid B but for the difference in the cationic lipid.

In some embodiments, the cationic lipid is GL-HEPES-E3-E12-DS-4-E10 (2-(4-(2-((4-(bis(2-hydroxydecyl)amino)butyl)disulfaneyl)ethyl)piperazin-1-yl)ethyl 4-(bis(2-hydroxydodecyl)amino)butanoate), which is a HEPES-based disulfide cationic lipid with a piperazine core, having the Formula IV:

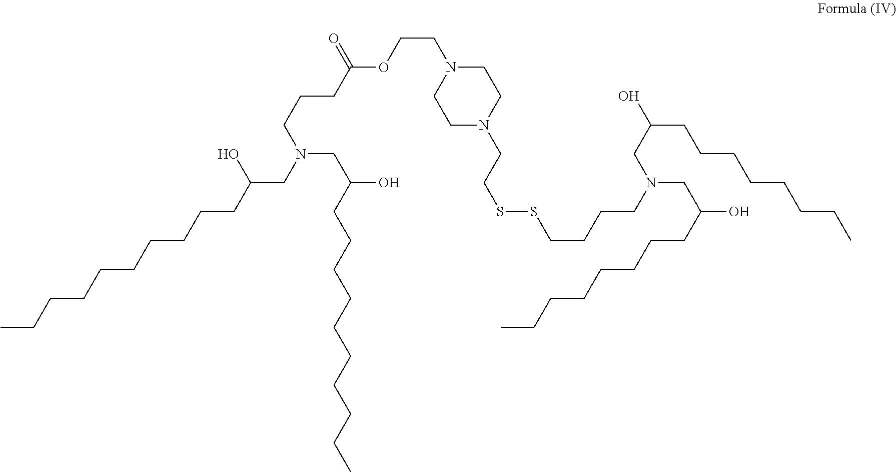 [SMILES: CCCCCCCCCCC(O)CN(CCCC(=O)OCCN1CCN(CCSSCCCCN(CC(O)CCCCCCCC)CC(O)CCCCCCCC)CC1)CC(O)CCCCCCCCCC]

An exemplary LNP formulation herein, Lipid D, contains GL-HEPES-E3-E12-DS-4-E10. Lipid D has the same composition as Lipid A or Lipid B but for the difference in the cationic lipid.

In some embodiments, the cationic lipid is GL-HEPES-E3-E12-DS-3-E14 (2-(4-(2-((3-(Bis(2-hydroxytetradecyl)amino)propyl)disulfaneyl)ethyl)piperazin-1-yl)ethyl 4-(bis(2-hydroxydodecyl)amino)butanoate), which is a HEPES-based disulfide cationic lipid with a piperazine core, having the Formula V:

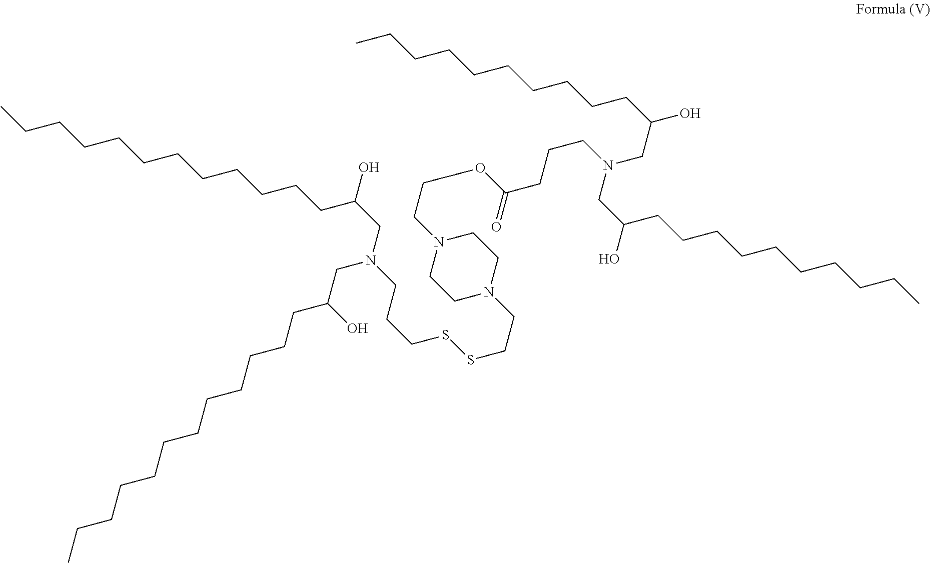 [SMILES: CCCCCCCCCCCCC(O)CN(CCCSSCCN1CCN(CCOC(=O)CCCN(CC(O)CCCCCCCCCC)CC(O)CCCCCCCCCC)CC1)CC(O)CCCCCCCCCCCC]

An exemplary LNP formulation herein, Lipid E, contains GL-HEPES-E3-E12-DS-3-E14. Lipid E has the same composition as Lipid A or Lipid B but for the difference in the cationic lipid.

The cationic lipids GL-HEPES-E3-E10-DS-3-E18-1 (III), GL-HEPES-E3-E12-DS-4-E10 (IV), and GL-HEPES-E3-E12-DS-3-E14 (V) can be synthesized according to the general procedure set out in Scheme 1:

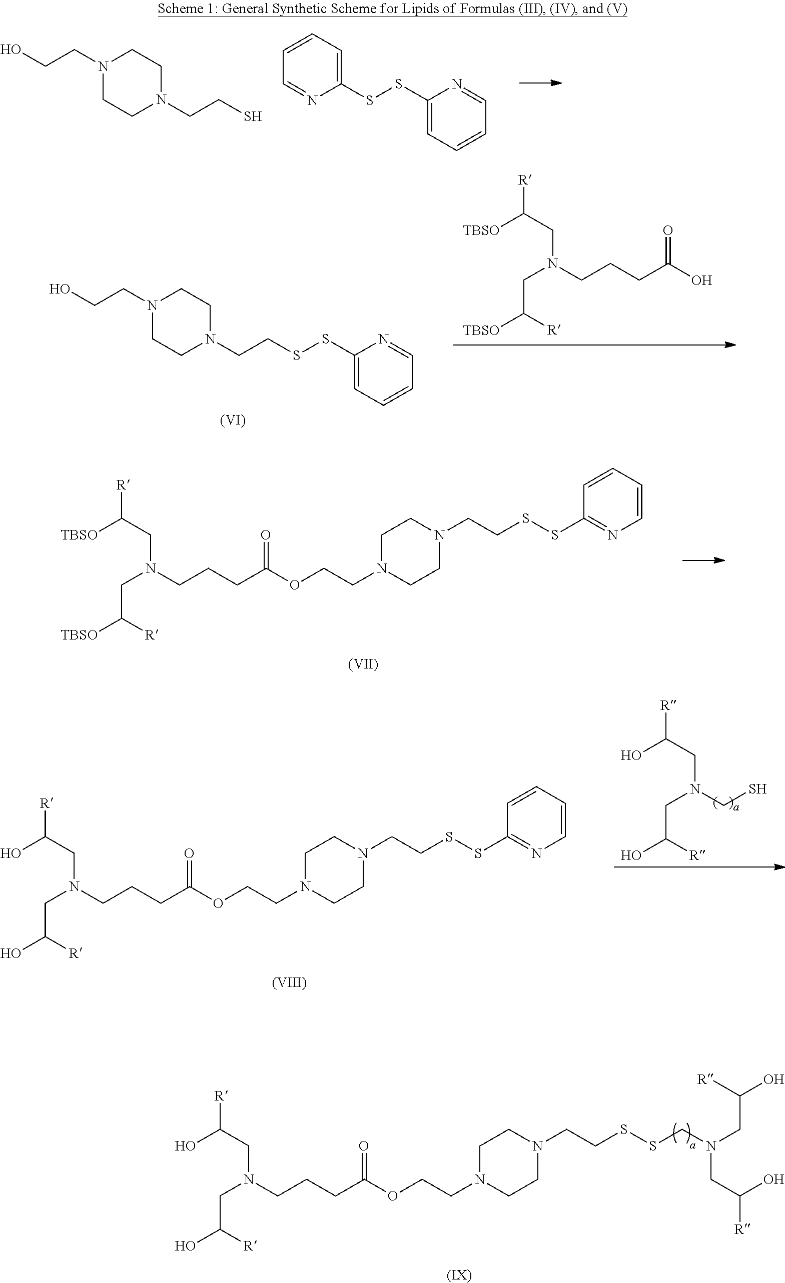

In some embodiments, the cationic lipid is MC3, having the Formula VI:

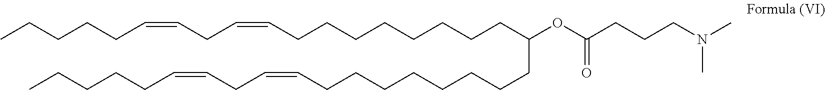 [SMILES: CCCCC/C=C\C/C=C\CCCCCCCCC(CCCCCCCC/C=C\C/C=C\CCCCC)OC(=O)CCCN(C)C]

In some embodiments, the cationic lipid is SM-102 (9-heptadecanyl 8-{(2-hydroxyethyl)[6-oxo-6-(undecyloxy)hexyl]amino}octanoate), having the Formula VII:

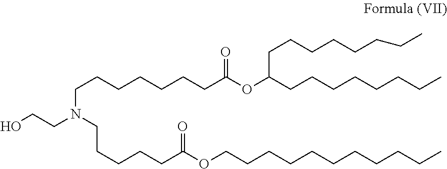 [SMILES: CCCCCCCCCCCOC(=O)CCCCCN(CCO)CCCCCCCC(=O)OC(CCCCCCCC)CCCCCCCC]

In some embodiments, the cationic lipid is ALC-0315 [(4-hydroxybutyl)azanediyl]di(hexane-6,1-diyl) bis(2-hexyldecanoate), having the Formula VIII:

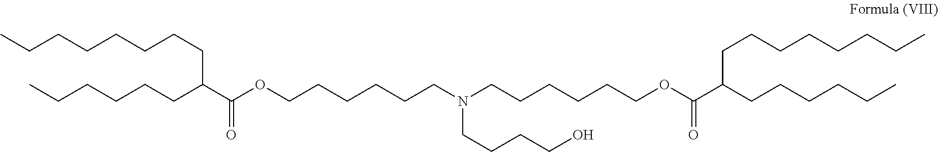 [SMILES: CCCCCCCCC(CCCCCC)C(=O)OCCCCCCN(CCCCO)CCCCCCOC(=O)C(CCCCCC)CCCCCCCC]

In some embodiments, the cationic lipid may be selected from the group comprising [ckkE10]/[OF-02], [(6Z,9Z,28Z,31Z)-heptatriaconta-6,9,28,31-tetraen-19-yl] 4-(dimethylamino)butanoate (D-Lin-MC3-DMA); 2,2-dilinoleyl-4-dimethylaminoethyl-[1,3]-dioxolane (DLin-KC2-DMA); 1,2-dilinoleyloxy-N,N-dimethyl-3-aminopropane (DLin-DMA); di((Z)-non-2-en-1-yl) 9-((4-(dimethylamino)butanoyl)oxy)heptadecanedioate (L319); 9-heptadecanyl 8-{(2-hydroxyethyl)[6-oxo-6-(undecyloxy)hexyl]amino}octanoate (SM-102); [(4-hydroxybutyl)azanediyl]di(hexane-6,1-diyl) bis(2-hexyldecanoate) (ALC-0315); [3-(dimethylamino)-2-[(Z)-octadec-9-enoyl]oxypropyl] (Z)-octadec-9-enoate (DODAP); 2,5-bis(3-aminopropylamino)-N-[2-[di(heptadecyl)amino]-2-oxoethyl]pentanamide (DOGS); [(3S,8S,9S,10R,13R,14S,17R)-10,13-dimethyl-17-[(2R)-6-methylheptan-2-yl]-2,3,4,7,8,9,11,12,14,15,16,17-dodecahydro-1H-cyclopenta[a]phenanthren-3-yl] N-[2-(dimethylamino)ethyl]carbamate (DC-Chol); tetrakis(8-methylnonyl) 3,3′,3″,3″′-(((methylazanediyl) bis(propane-3,1 diyl))bis (azanetriyl))tetrapropionate (3060110); decyl (2-(dioctylammonio)ethyl) phosphate (9A1P9); ethyl 5,5-di((Z)-heptadec-8-en-1-yl)-1-(3-(pyrrolidin-1-yl)propyl)-2,5-dihydro-1H-imidazole-2-carboxylate (A2-Iso5-2DC18); bis(2-(dodecyldisulfanyl)ethyl) 3,3′-((3-methyl-9-oxo-10-oxa-13,14-dithia-3,6-diazahexacosyl)azanediyl)dipropionate (BAME-016B); 1,1′-((2-(4-(2-((2-(bis(2-hydroxydodecyl)amino)ethyl) (2-hydroxydodecyl)amino)ethyl) piperazin-1-yl)ethyl)azanediyl) bis(dodecan-2-ol) (C12-200); 3,6-bis(4-(bis(2-hydroxydodecyl)amino)butyl)piperazine-2,5-dione (cKK-E12); hexa(octan-3-yl) 9,9′,9″,9″′,9″″,9″″′-((((benzene-1,3,5-tricarbonyl)yris(azanediyl)) tris (propane-3,1-diyl)) tris(azanetriyl))hexanonanoate (FTT5); (((3,6-dioxopiperazine-2,5-diyl)bis(butane-4, 1-diyl))bis(azanetriyl))tetrakis(ethane-2,1-diyl) (9Z,9′Z,9″Z,9″′Z,12Z,12′Z,12″Z,12″′Z)-tetrakis (octadeca-9,12-dienoate) (OF-Deg-Lin); TT3; N¹,N³,N⁵-tris(3-(didodecylamino)propyl)benzene-1,3,5-tricarboxamide; N1-[2-((1S)-1-[(3-aminopropyl)amino]-4-[di(3-aminopropyl)amino]butylcarboxamido)ethyl]-3,4-di[oleyloxy]-benzamide (MVL5); heptadecan-9-yl 8-((2-hydroxyethyl)(8-(nonyloxy)-8-oxooctyl)amino)octanoate (Lipid 5); and combinations thereof.

In some embodiments, the cationic lipid is biodegradable.

In some embodiments, the cationic lipid is not biodegradable.

In some embodiments, the cationic lipid is cleavable.

In some embodiments, the cationic lipid is not cleavable.

Cationic lipids are described in further detail in Dong et al. (PNAS. 111(11):3955-60. 2014); Fenton et al. (Adv Mater. 28:2939. 2016); U.S. Pat. Nos. 9,512,073; and 10,201,618, each of which is incorporated herein by reference.

B. PEGylated Lipids

The PEGylated lipid component provides control over particle size and stability of the nanoparticle. The addition of such components may prevent complex aggregation and provide a means for increasing circulation lifetime and increasing the delivery of the lipid-nucleic acid pharmaceutical composition to target tissues (Klibanov et al. FEBS Letters 268(1):235-7. 1990). These components may be selected to rapidly exchange out of the pharmaceutical composition in vivo (see, e.g., U.S. Pat. No. 5,885,613).

Contemplated PEGylated lipids include, but are not limited to, a polyethylene glycol (PEG) chain of up to 5 kDa in length covalently attached to a lipid with alkyl chain(s) of C₆-C₂₀ (e.g., C₈, C₁₀, C₁₂, C₁₄, C₁₆, or Cis) length, such as a derivatized ceramide (e.g., N-octanoyl-sphingosine-1-[succinyl(methoxypolyethylene glycol)] (C8 PEG ceramide)). In some embodiments, the PEGylated lipid is 1,2-dimyristoyl-rac-glycero-3-methoxypolyethylene glycol (DMG-PEG); 1,2-distearoyl-sn-glycero-3-phosphoethanolamine-polyethylene glycol (DSPE-PEG); 1,2-dilauroyl-sn-glycero-3-phosphoethanolamine-polyethylene glycol (DLPE-PEG); or 1,2-distearoyl-rac-glycero-polyethelene glycol (DSG-PEG), PEG-DAG; PEG-PE; PEG-S-DAG; PEG-S-DMG; PEG-cer; a PEG-dialkyoxypropylcarbamate; 2-[(polyethylene glycol)-2000]-N,N-ditetradecylacetamide (ALC-0159); and combinations thereof.

In certain embodiments, the PEG has a high molecular weight, e.g., 2000-2400 g/mol. In certain embodiments, the PEG is PEG2000 (or PEG-2K). In certain embodiments, the PEGylated lipid herein is DMG-PEG2000, DSPE-PEG2000, DLPE-PEG2000, DSG-PEG2000, C8 PEG2000, or ALC-0159 (2-[(polyethylene glycol)-2000]-N,N-ditetradecylacetamide). In certain embodiments, the PEGylated lipid herein is DMG-PEG2000.

C. Cholesterol-Based Lipids

The cholesterol component provides stability to the lipid bilayer structure within the nanoparticle. In some embodiments, the LNPs comprise one or more cholesterol-based lipids. Suitable cholesterol-based lipids include, for example: DC-Choi (N,N-dimethyl-N-ethylcarboxamidocholesterol), 1,4-bis(3-N-oleylamino-propyl)piperazine (Gao et al., Biochem Biophys Res Comm. (1991) 179:280; Wolf et al., BioTechniques (1997) 23:139; U.S. Pat. No. 5,744,335), imidazole cholesterol ester (“ICE”; WO2011/068810), sitosterol (22,23-dihydrostigmasterol), β-sitosterol, sitostanol, fucosterol, stigmasterol (stigmasta-5,22-dien-3-ol), ergosterol; desmosterol (3β-hydroxy-5,24-cholestadiene); lanosterol (8,24-lanostadien-3b-ol); 7-dehydrocholesterol (Δ5,7-cholesterol); dihydrolanosterol (24,25-dihydrolanosterol); zymosterol (5α-cholesta-8,24-dien-3β-ol); lathosterol (5α-cholest-7-en-3β-ol); diosgenin ((3β,25R)-spirost-5-en-3-ol); campesterol (campest-5-en-3β-ol); campestanol (5a-campestan-3b-ol); 24-methylene cholesterol (5,24(28)-cholestadien-24-methylen-3β-ol); cholesteryl margarate (cholest-5-en-3β-yl heptadecanoate); cholesteryl oleate; cholesteryl stearate and other modified forms of cholesterol. In some embodiments, the cholesterol-based lipid used in the LNPs is cholesterol.

D. Helper Lipids

A helper lipid enhances the structural stability of the LNP and helps the LNP in endosome escape. It improves uptake and release of the mRNA drug payload. In some embodiments, the helper lipid is a zwitterionic lipid, which has fusogenic properties for enhancing uptake and release of the drug payload. Examples of helper lipids are 1,2-dioleoyl-SN-glycero-3-phosphoethanolamine (DOPE); 1,2-distearoyl-sn-glycero-3-phosphocholine (DSPC); 1,2-dioleoyl-sn-glycero-3-phospho-L-serine (DOPS); 1,2-dielaidoyl-sn-glycero-3-phosphoethanolamine (DEPE); and 1,2-dioleoyl-sn-glycero-3-phosphocholine (DPOC), dipalmitoylphosphatidylcholine (DPPC), DMPC, 1,2-dilauroyl-sn-glycero-3-phosphocholine (DLPC), 1,2-Distearoylphosphatidylethanolamine (DSPE), and 1,2-dilauroyl-sn-glycero-3-phosphoethanolamine (DLPE).

Other exemplary helper lipids are dioleoylphosphatidylcholine (DOPC), dioleoylphosphatidylglycerol (DOPG), dipalmitoylphosphatidylglycerol (DPPG), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), phosphatidylserine, sphingolipids, sphingomyelins, ceramides, cerebrosides, gangliosides, 16-O-monomethyl PE, 16-O-dimethyl PE, 18-1-trans PE, 1-stearoyl-2-oleoyl-phosphatidyethanolamine (SOPE), or a combination thereof. In certain embodiments, the helper lipid is DOPE. In certain embodiments, the helper lipid is DSPC.

In various embodiments, the present LNPs comprise (i) a cationic lipid selected from OF-02, cKK-E10, GL-HEPES-E3-E10-DS-3-E18-1, GL-HEPES-E3-E12-DS-4-E10, or GL-HEPES-E3-E12-DS-3-E14; (ii) DMG-PEG2000; (iii) cholesterol; and (iv) DOPE.

E. Molar Ratios of the Lipid Components

The molar ratios of the above components are important for the LNPs' effectiveness in delivering mRNA. The molar ratio of the cationic lipid, the PEGylated lipid, the cholesterol-based lipid, and the helper lipid is A: B: C: D, where A+B+C+D=100%. In some embodiments, the molar ratio of the cationic lipid in the LNPs relative to the total lipids (i.e., A) is 35-55%, such as 35-50% (e.g., 38-42% such as 40%, or 45-50%). In some embodiments, the molar ratio of the PEGylated lipid component relative to the total lipids (i.e., B) is 0.25-2.75% (e.g., 1-2% such as 1.5%). In some embodiments, the molar ratio of the cholesterol-based lipid relative to the total lipids (i.e., C) is 20-50% (e.g., 27-30% such as 28.5%, or 38-43%). In some embodiments, the molar ratio of the helper lipid relative to the total lipids (i.e., D) is 5-35% (e.g., 28-32% such as 30%, or 8-12%, such as 10%). In some embodiments, the (PEGylated lipid+cholesterol) components have the same molar amount as the helper lipid. In some embodiments, the LNPs contain a molar ratio of the cationic lipid to the helper lipid that is more than 1.

In certain embodiments, the LNP of the disclosure comprises:

a cationic lipid at a molar ratio of 35% to 55% or 40% to 50% (e.g., a cationic lipid at a molar ratio of 35%, 36%, 37%, 38%, 39%, 40%, 41% 42%, 43%, 44%, 45%, 46%, 47%, 48%, 49%, 50%, 51%, 52%, 53%, 54%, or 55%);

a polyethylene glycol (PEG) conjugated (PEGylated) lipid at a molar ratio of 0.25% to 2.75% or 1.00% to 2.00% (e.g., a PEGylated lipid at a molar ratio of 0.25%, 0.50%, 0.75%, 1.00%, 1.25%, 1.50%, 1.75%, 2.00%, 2.25%, 2.50%, or 2.75%);

a cholesterol-based lipid at a molar ratio of 20% to 50%, 25% to 45%, or 28.5% to 43% (e.g., a cholesterol-based lipid at a molar ratio of 20%, 21%, 22%, 23%, 24%, 25%, 26%, 27%, 28%, 29%, 30%, 31%, 32%, 33%, 34%, 35%, 36%, 37%, 38%, 39%, 40%, 41% 42%, 43%, 44%, 45%, 46%, 47%, 48%, 49%, or 50%); and

a helper lipid at a molar ratio of 5% to 35%, 8% to 30%, or 10% to 30% (e.g., a helper lipid at a molar ratio of 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, or 35%),

wherein all of the molar ratios are relative to the total lipid content of the LNP.

In certain embodiments, the LNP comprises: a cationic lipid at a molar ratio of 40%; a PEGylated lipid at a molar ratio of 1.5%; a cholesterol-based lipid at a molar ratio of 28.5%; and a helper lipid at a molar ratio of 30%.

In certain embodiments, the PEGylated lipid is dimyristoyl-PEG2000 (DMG-PEG2000).

In various embodiments, the cholesterol-based lipid is cholesterol.

In some embodiments, the helper lipid is 1,2-dioleoyl-SN-glycero-3-phosphoethanolamine (DOPE).

In certain embodiments, the LNP comprises: OF-02 at a molar ratio of 35% to 55%; DMG-PEG2000 at a molar ratio of 0.25% to 2.75%; cholesterol at a molar ratio of 20% to 50%; and DOPE at a molar ratio of 5% to 35%.

In certain embodiments, the LNP comprises: cKK-E10 at a molar ratio of 35% to 55%; DMG-PEG2000 at a molar ratio of 0.25% to 20.75%; cholesterol at a molar ratio of 20% to 50%; and DOPE at a molar ratio of 5% to 35%.

In certain embodiments, the LNP comprises: GL-HEPES-E3-E10-DS-3-E18-1 at a molar ratio of 35% to 55%; DMG-PEG2000 at a molar ratio of 0.25% to 20.75%; cholesterol at a molar ratio of 20% to 50%; and DOPE at a molar ratio of 5% to 35%.

In certain embodiments, the LNP comprises: GL-HEPES-E3-E12-DS-4-E10 at a molar ratio of 35% to 55%; DMG-PEG2000 at a molar ratio of 0.25% to 20.75%; cholesterol at a molar ratio of 20% to 50%; and DOPE at a molar ratio of 5% to 35%.

In certain embodiments, the LNP comprises: GL-HEPES-E3-E12-DS-3-E14 at a molar ratio of 35% to 55%; DMG-PEG2000 at a molar ratio of 0.25% to 2.75%; cholesterol at a molar ratio of 20% to 50%; and DOPE at a molar ratio of 5% to 35%.

In certain embodiments, the LNP comprises: SM-102 at a molar ratio of 35% to 55%; DMG-PEG2000 at a molar ratio of 0.25% to 2.75%; cholesterol at a molar ratio of 20% to 50%; and DSPC at a molar ratio of 5% to 35%.

In certain embodiments, the LNP comprises: ALC-0315 at a molar ratio of 35% to 55%; ALC-0159 at a molar ratio of 0.25% to 2.75%; cholesterol at a molar ratio of 20% to 50%; and DSPC at a molar ratio of 5% to 35%.

In certain embodiments, the LNP comprises: OF-02 at a molar ratio of 40%; DMG-PEG2000 at a molar ratio of 1.5%; cholesterol at a molar ratio of 28.5%; and DOPE at a molar ratio of 30%. This LNP formulation is designated “Lipid A” herein.

In certain embodiments, the LNP comprises: cKK-E10 at a molar ratio of 40%; DMG-PEG2000 at a molar ratio of 1.5%; cholesterol at a molar ratio of 28.5%; and DOPE at a molar ratio of 30%. This LNP formulation is designated “Lipid B” herein.

In certain embodiments, the LNP comprises: GL-HEPES-E3-E10-DS-3-E18-1 at a molar ratio of 40%; DMG-PEG2000 at a molar ratio of 1.5%; cholesterol at a molar ratio of 28.5%; and DOPE at a molar ratio of 30%. This LNP formulation is designated “Lipid C” herein.

In certain embodiments, the LNP comprises: GL-HEPES-E3-E12-DS-4-E10 (at a molar ratio of 40%; DMG-PEG2000 at a molar ratio of 1.5%; cholesterol at a molar ratio of 28.5%; and DOPE at a molar ratio of 30%. This LNP formulation is designated “Lipid D” herein.

In certain embodiments, the LNP comprises: GL-HEPES-E3-E12-DS-3-E14 at a molar ratio of 40%; DMG-PEG2000 at a molar ratio of 1.5%; cholesterol at a molar ratio of 28.5%; and DOPE at a molar ratio of 30%. This LNP formulation is designated “Lipid E” herein.

In certain embodiments, the LNP comprises: 9-heptadecanyl 8-{(2-hydroxyethyl)[6-oxo-6-(undecyloxy)hexyl]amino}octanoate (SM-102) at a molar ratio of 50%; 1,2-distearoyl-sn-glycero-3-phosphocholine (DSPC) at a molar ratio of 10%; cholesterol at a molar ratio of 38.5%; and 1,2-dimyristoyl-rac-glycero-3-methoxypolyethylene glycol-2000 (DMG-PEG2000) at a molar ratio of 1.5%.

In certain embodiments, the LNP comprises: (4-hydroxybutyl)azanediyl]di(hexane-6,1-diyl) bis(2-hexyldecanoate) (ALC-0315) at a molar ratio of 46.3%; 1,2-distearoyl-sn-glycero-3-phosphocholine (DSPC) at a molar ratio of 9.4%; cholesterol at a molar ratio of 42.7%; and 2-[(polyethylene glycol)-2000]-N,N-ditetradecylacetamide (ALC-0159) at a molar ratio of 1.6%.

In certain embodiments, the LNP comprises: (4-hydroxybutyl)azanediyl]di(hexane-6,1-diyl) bis(2-hexyldecanoate) (ALC-0315) at a molar ratio of 47.4%; 1,2-distearoyl-sn-glycero-3-phosphocholine (DSPC) at a molar ratio of 10%; cholesterol at a molar ratio of 40.9%; and 2-[(polyethylene glycol)-2000]-N,N-ditetradecylacetamide (ALC-0159) at a molar ratio of 1.7%.

To calculate the actual amount of each lipid to be put into an LNP formulation, the molar amount of the cationic lipid is first determined based on a desired N/P ratio, where N is the number of nitrogen atoms in the cationic lipid and P is the number of phosphate groups in the mRNA to be transported by the LNP. Next, the molar amount of each of the other lipids is calculated based on the molar amount of the cationic lipid and the molar ratio selected. These molar amounts are then converted to weights using the molecular weight of each lipid

F. Active Ingredients of the LNPs

The active ingredient of the present LNP vaccine composition is an mRNA that encodes an influenza antigen.

Where desired, the LNP may be multi-valent. In some embodiments, the LNP may carry mRNAs that encode more than one influenza antigen, such as two, three, four, five, six, seven, or eight antigens. For example, the LNP may carry multiple mRNA, each encoding a different influenza antigen; or carry a polycistronic mRNA that can be translated into more than one influenza antigen (e.g., each antigen-coding sequence is separated by a nucleotide linker encoding a self-cleaving peptide such as a 2A peptide). An LNP carrying different mRNA typically comprises (encapsulate) multiple copies of each mRNA. For example, an LNP carrying or encapsulating two different mRNA typically carries multiple copies of each of the two different mRNA.

In some embodiments, a single LNP formulation may comprise multiple kinds (e.g., two, three, four, five, six, seven, eight, nine, ten, or more) of LNPs, each kind carrying a different mRNA.

In some embodiments, the multi-valent LNP vaccines contain mRNA molecules encoding polypeptides derived from eight influenza viral proteins selected from hemagglutinin (e.g., hemagglutinin 1 (HA1) and hemagglutinin 2 (HA2)), neuraminidase (NA), nucleoprotein (NP), matrix protein 1 (M1), matrix protein 2 (M2), nonstructural protein 1 (NS1), and non-structural protein 2 (NS2). In further embodiments, the multi-valent LNP vaccines containing eight mRNA encoding antigenic polypeptides derived from an HA protein, from an NA protein, and from both HA and NA proteins. In some embodiments, the mRNA encoding antigenic polypeptides are derived from different influenza strains.

In certain embodiments, the composition may comprise one or more mRNA encoding antigens of influenza A, B and C viruses. In one embodiment, the composition may comprise one or more mRNA encoding HA and/or NA antigens of influenza A and influenza B viruses. In one embodiment, the HA antigens of influenza A viruses are selected from subtypes H1, H2, H3, H4, H5, H6, H7, H8, H9, H10, H11, H12, H13, H14, H15, H16, H17, and H18. In one embodiment, the NA antigens of influenza A viruses are selected from subtypes N1, N2, N3, N4, N5, N6, N7, N8, N9, N10, and N11. In one embodiment, the HA and NA antigens of influenza B viruses are from the influenza B/Yamagata lineage. In one embodiment, the HA and NA antigens of influenza B viruses are from the influenza B/Victoria lineage. In some embodiments, the one or more HA and NA antigens are from influenza virus strains recommended by the World Health Organization (WHO) in their annual recommendation for influenza vaccine formulations.

In certain embodiments, at least one of the one or more influenza virus proteins comprises an influenza virus HA protein and/or an influenza virus NA protein having a molecular sequence identified or designed from a machine learning model, and in certain embodiments, at least one of the one or more ribonucleic acid molecules encode one or more influenza virus proteins having a molecular sequence identified or designed from a machine learning model.

In one embodiment, the composition comprises one mRNA encoding an H3 HA antigen, one mRNA encoding an H1 HA antigen, one mRNA encoding an HA antigen from the influenza B/Yamagata lineage, and one mRNA encoding an HA antigen from the influenza B/Victoria lineage.

In one embodiment, the composition comprises one mRNA encoding an H3 HA antigen, one mRNA encoding an N2 NA antigen, one mRNA encoding an H1 HA antigen, one mRNA encoding an N1 NA antigen, one mRNA encoding an HA antigen from the influenza B/Yamagata lineage, one mRNA encoding an NA antigen from the influenza B/Yamagata lineage, one mRNA encoding an HA antigen from the influenza B/Victoria lineage, and one mRNA encoding an NA antigen from the influenza B/Victoria lineage.

In an embodiment, the composition comprises further comprise one or more mRNA encoding a machine learning influenza virus HA having a molecular sequence identified or designed from a machine learning model, wherein the one or more machine learning influenza virus HA may be selected from an H1 HA, an H3 HA, an HA from a B/Victoria lineage, an HA from a B/Yamagata lineage, or a combination thereof.

When selecting one or more machine learning influenza virus HAs, any machine learning algorithm may be used. For example, envisioned herein are any of the machine learning algorithms and methods disclosed in PCT Application Nos. WO 2021/080990 A1, entitled Systems and Methods for Designing Vaccines, and WO 2021/080999 A1, entitled Systems and Methods for Predicting Biological Responses, both of which are incorporated by reference in their entireties herein.

The mRNA may be unmodified (i.e., containing only natural ribonucleotides A, U, C, and/or G linked by phosphodiester bonds), or chemically modified (e.g., including nucleotide analogs such as pseudouridines (e.g., N-1-methyl pseudouridine), 2′-fluoro ribonucleotides, and 2′-methoxy ribonucleotides, and/or phosphorothioate bonds). The mRNA molecule may comprise a 5′ cap and a polyA tail.

G. Buffer and Other Components

To stabilize the nucleic acid and/or LNPs (e.g., to prolong the shelf-life of the vaccine product), to facilitate administration of the LNP pharmaceutical composition, and/or to enhance in vivo expression of the nucleic acid, the nucleic acid and/or LNP can be formulated in combination with one or more carriers, targeting ligands, stabilizing reagents (e.g., preservatives and antioxidants), and/or other pharmaceutically acceptable excipients. Examples of such excipients are parabens, thimerosal, thiomersal, chlorobutanol, benzalkonium chloride, and chelators (e.g., EDTA).

The LNP compositions of the present disclosure can be provided as a frozen liquid form or a lyophilized form. A variety of cryoprotectants may be used, including, without limitations, sucrose, trehalose, glucose, mannitol, mannose, dextrose, and the like. The cryoprotectant may constitute 5-30% (w/v) of the LNP composition. In some embodiments, the LNP composition comprises trehalose, e.g., at 5-30% (e.g., 10%) (w/v). Once formulated with the cryoprotectant, the LNP compositions may be frozen (or lyophilized and cryopreserved) at −20° C. to −80° C.

The LNP compositions may be provided to a patient in an aqueous buffered solution—thawed if previously frozen, or if previously lyophilized, reconstituted in an aqueous buffered solution at bedside. The buffered solution preferably is isotonic and suitable for e.g., intramuscular or intradermal injection. In some embodiments, the buffered solution is a phosphate-buffered saline (PBS).

### II. RNA

The present LNP vaccine compositions of the disclosure may comprise an RNA molecule (e.g., mRNA) that encodes an antigen of interest. The RNA molecule of the present disclosure may comprise at least one ribonucleic acid (RNA) comprising an ORF encoding an antigen of interest. In certain embodiments, the RNA is a messenger RNA (mRNA) comprising an ORF encoding an antigen of interest. In certain embodiments, the RNA (e.g., mRNA) further comprises at least one 5′ UTR, 3′ UTR, a poly(A) tail, and/or a 5′ cap.

II. A. 5′ Cap

An mRNA 5′ cap can provide resistance to nucleases found in most eukaryotic cells and promote translation efficiency. Several types of 5′ caps are known. A 7-methylguanosine cap (also referred to as “m⁷G” or “Cap-0”), comprises a guanosine that is linked through a 5′-5′-triphosphate bond to the first transcribed nucleotide.

A 5′ cap is typically added as follows: first, an RNA terminal phosphatase removes one of the terminal phosphate groups from the 5′ nucleotide, leaving two terminal phosphates; guanosine triphosphate (GTP) is then added to the terminal phosphates via a guanylyl transferase, producing a 5′5′5 triphosphate linkage; and the 7-nitrogen of guanine is then methylated by a methyltransferase. Examples of cap structures include, but are not limited to, m7G(5′)ppp, (5′(A,G(5′)ppp(5′)A, and G(5′)ppp(5′)G. Additional cap structures are described in U.S. Publication No. US 2016/0032356 and U.S. Publication No. US 2018/0125989, which are incorporated herein by reference.

5′-capping of polynucleotides may be completed concomitantly during the in vitro-transcription reaction using the following chemical RNA cap analogs to generate the 5′-guanosine cap structure according to manufacturer protocols: 3′-O-Me-m7G(5′)ppp(5′)G (the ARCA cap); G(5′)ppp(5′)A; G(5′)ppp(5′)G; m7G(5′)ppp(5′)A; m7G(5′)ppp(5′)G; m7G(5′)ppp(5′)(2′OMeA)pG; m7G(5′)ppp(5′)(2′OMeA)pU; m7G(5′)ppp(5′)(2′OMeG)pG (New England BioLabs, Ipswich, Mass.; TriLink Biotechnologies). 5′-capping of modified RNA may be completed post-transcriptionally using a vaccinia virus capping enzyme to generate the Cap 0 structure: m7G(5′)ppp(5′)G. Cap 1 structure may be generated using both vaccinia virus capping enzyme and a 2′-O methyl-transferase to generate: m7G(5′)ppp(5′)G-2′-O-methyl. Cap 2 structure may be generated from the Cap 1 structure followed by the 2′-O-methylation of the 5′-antepenultimate nucleotide using a 2′-O methyl-transferase. Cap 3 structure may be generated from the Cap 2 structure followed by the 2′-O-methylation of the 5′-preantepenultimate nucleotide using a 2′-O methyl-transferase.

In certain embodiments, the mRNA of the disclosure comprises a 5′ cap selected from the group consisting of 3′-O-Me-m7G(5′)ppp(5′)G (the ARCA cap), G(5′)ppp(5′)A, G(5′)ppp(5′)G, m7G(5′)ppp(5′)A, m7G(5′)ppp(5′)G, m7G(5′)ppp(5′)(2′OMeA)pG, m7G(5′)ppp(5′)(2′OMeA)pU, and m7G(5′)ppp(5′)(2′OMeG)pG.

In certain embodiments, the mRNA of the disclosure comprises a 5′ cap of:

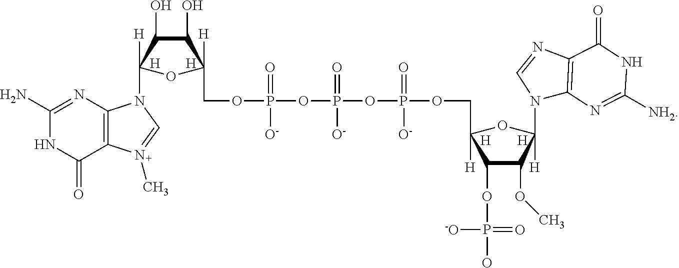

II. B. Untranslated Region (UTR)

In some embodiments, the mRNA of the disclosure includes a 5′ and/or 3′ untranslated region (UTR). In mRNA, the 5′ UTR starts at the transcription start site and continues to the start codon but does not include the start codon. The 3′ UTR starts immediately following the stop codon and continues until the transcriptional termination signal.

In some embodiments, the mRNA disclosed herein may comprise a 5′ UTR that includes one or more elements that affect an mRNA's stability or translation. In some embodiments, a 5′ UTR may be about 10 to 5,000 nucleotides in length. In some embodiments, a 5′ UTR may be about 50 to 500 nucleotides in length. In some embodiments, the 5′ UTR is at least about 10 nucleotides in length, about 20 nucleotides in length, about 30 nucleotides in length, about 40 nucleotides in length, about 50 nucleotides in length, about 100 nucleotides in length, about 150 nucleotides in length, about 200 nucleotides in length, about 250 nucleotides in length, about 300 nucleotides in length, about 350 nucleotides in length, about 400 nucleotides in length, about 450 nucleotides in length, about 500 nucleotides in length, about 550 nucleotides in length, about 600 nucleotides in length, about 650 nucleotides in length, about 700 nucleotides in length, about 750 nucleotides in length, about 800 nucleotides in length, about 850 nucleotides in length, about 900 nucleotides in length, about 950 nucleotides in length, about 1,000 nucleotides in length, about 1,500 nucleotides in length, about 2,000 nucleotides in length, about 2,500 nucleotides in length, about 3,000 nucleotides in length, about 3,500 nucleotides in length, about 4,000 nucleotides in length, about 4,500 nucleotides in length or about 5,000 nucleotides in length.

In some embodiments, the mRNA disclosed herein may comprise a 3′ UTR comprising one or more of a polyadenylation signal, a binding site for proteins that affect an mRNA's stability of location in a cell, or one or more binding sites for miRNAs. In some embodiments, a 3′ UTR may be 50 to 5,000 nucleotides in length or longer. In some embodiments, a 3′ UTR may be 50 to 1,000 nucleotides in length or longer. In some embodiments, the 3′ UTR is at least about 50 nucleotides in length, about 100 nucleotides in length, about 150 nucleotides in length, about 200 nucleotides in length, about 250 nucleotides in length, about 300 nucleotides in length, about 350 nucleotides in length, about 400 nucleotides in length, about 450 nucleotides in length, about 500 nucleotides in length, about 550 nucleotides in length, about 600 nucleotides in length, about 650 nucleotides in length, about 700 nucleotides in length, about 750 nucleotides in length, about 800 nucleotides in length, about 850 nucleotides in length, about 900 nucleotides in length, about 950 nucleotides in length, about 1,000 nucleotides in length, about 1,500 nucleotides in length, about 2,000 nucleotides in length, about 2,500 nucleotides in length, about 3,000 nucleotides in length, about 3,500 nucleotides in length, about 4,000 nucleotides in length, about 4,500 nucleotides in length, or about 5,000 nucleotides in length.

In some embodiments, the mRNA disclosed herein may comprise a 5′ or 3′ UTR that is derived from a gene distinct from the one encoded by the mRNA transcript (i.e., the UTR is a heterologous UTR).

In certain embodiments, the 5′ and/or 3′ UTR sequences can be derived from mRNA which are stable (e.g., globin, actin, GAPDH, tubulin, histone, or citric acid cycle enzymes) to increase the stability of the mRNA. For example, a 5′ UTR sequence may include a partial sequence of a CMV immediate-early 1 (IE1) gene, or a fragment thereof, to improve the nuclease resistance and/or improve the half-life of the mRNA. Also contemplated is the inclusion of a sequence encoding human growth hormone (hGH), or a fragment thereof, to the 3′ end or untranslated region of the mRNA. Generally, these modifications improve the stability and/or pharmacokinetic properties (e.g., half-life) of the mRNA relative to their unmodified counterparts, and include, for example, modifications made to improve such mRNA resistance to in vivo nuclease digestion.

Exemplary 5′ UTRs include a sequence derived from a CMV immediate-early 1 (IE1) gene (U.S. Publication Nos. 2014/0206753 and 2015/0157565, each of which is incorporated herein by reference), or the sequence GGGAUCCUACC (SEQ ID NO: 22) (U.S. Publication No. 2016/0151409, incorporated herein by reference).

In various embodiments, the 5′ UTR may be derived from the 5′ UTR of a TOP gene. TOP genes are typically characterized by the presence of a 5′-terminal oligopyrimidine (TOP) tract. Furthermore, most TOP genes are characterized by growth-associated translational regulation. However, TOP genes with a tissue specific translational regulation are also known. In certain embodiments, the 5′ UTR derived from the 5′ UTR of a TOP gene lacks the 5′ TOP motif (the oligopyrimidine tract) (e.g., U.S. Publication Nos. 2017/0029847, 2016/0304883, 2016/0235864, and 2016/0166710, each of which is incorporated herein by reference).

In certain embodiments, the 5′ UTR is derived from a ribosomal protein Large 32 (L32) gene (U.S. Publication No. 2017/0029847, supra).

In certain embodiments, the 5′ UTR is derived from the 5′ UTR of an hydroxysteroid (17-b) dehydrogenase 4 gene (HSD17B4) (U.S. Publication No. 2016/0166710, supra).

In certain embodiments, the 5′ UTR is derived from the 5′ UTR of an ATP5A1 gene (U.S. Publication No. 2016/0166710, supra).

In some embodiments, an internal ribosome entry site (IRES) is used instead of a 5′ UTR.

In some embodiments, the 5′UTR comprises a nucleic acid sequence set forth in SEQ ID NO: 19 and reproduced below:

(SEQ ID NO: 19)
GGACAGAUCGCCUGGAGACGCCAUCCACGCUGUUUUGACCUCCAUAG
AAGACACCGGGACCGAUCCAGCCUCCGCGGCCGGGAACGGUGCAUUG
GAACGCGGAUUCCCCGUGCCAAGAGUGACUCACCGUCCUUGACACG.

In some embodiments, the 3′UTR comprises a nucleic acid sequence set forth in SEQ ID NO: 20 and reproduced below:

(SEQ ID NO: 20)
CGGGUGGCAUCCCUGUGACCCCUCCCCAGUGCCUCUCCUGGCCCUGG
AAGUUGCCACUCCAGUGCCCACCAGCCUUGUCCUAAUAAAAUUAAGU
UGCAUC.

The 5′ UTR and 3′UTR are described in further detail in WO2012/075040, incorporated herein by reference.

II. C. Polyadenylated Tail

As used herein, the terms “poly(A) sequence,” “poly(A) tail,” and “poly(A) region” refer to a sequence of adenosine nucleotides at the 3′ end of the mRNA molecule. The poly(A) tail may confer stability to the mRNA and protect it from exonuclease degradation. The poly(A) tail may enhance translation. In some embodiments, the poly(A) tail is essentially homopolymeric. For example, a poly(A) tail of 100 adenosine nucleotides may have essentially a length of 100 nucleotides. In certain embodiments, the poly(A) tail may be interrupted by at least one nucleotide different from an adenosine nucleotide (e.g., a nucleotide that is not an adenosine nucleotide). For example, a poly(A) tail of 100 adenosine nucleotides may have a length of more than 100 nucleotides (comprising 100 adenosine nucleotides and at least one nucleotide, or a stretch of nucleotides, that are different from an adenosine nucleotide). In certain embodiments, the poly(A) tail comprises the sequence

(SEQ ID NO: 23)
AAAAAAAAAAAAAAAAAAAAAAAAAAAAAAGCAUAUGACUAAAAAAAAAA
AAAAAAAAAAAAAAAAAAAAAAAAAAAAAAAAAAAAAAAAAAAAAAAAAA
AAAAAAAAAA.

The “poly(A) tail,” as used herein, typically relates to RNA. However, in the context of the disclosure, the term likewise relates to corresponding sequences in a DNA molecule (e.g., a “poly(T) sequence”).

The poly(A) tail may comprise about 10 to about 500 adenosine nucleotides, about 10 to about 200 adenosine nucleotides, about 40 to about 200 adenosine nucleotides, or about 40 to about 150 adenosine nucleotides. The length of the poly(A) tail may be at least about 10, 50, 75, 100, 150, 200, 250, 300, 350, 400, 450, or 500 adenosine nucleotides.

In some embodiments where the nucleic acid is an RNA, the poly(A) tail of the nucleic acid is obtained from a DNA template during RNA in vitro transcription. In certain embodiments, the poly(A) tail is obtained in vitro by common methods of chemical synthesis without being transcribed from a DNA template. In various embodiments, poly(A) tails are generated by enzymatic polyadenylation of the RNA (after RNA in vitro transcription) using commercially available polyadenylation kits and corresponding protocols, or alternatively, by using immobilized poly(A)polymerases, e.g., using methods and means as described in WO2016/174271.

The nucleic acid may comprise a poly(A) tail obtained by enzymatic polyadenylation, wherein the majority of nucleic acid molecules comprise about 100 (+/−20) to about 500 (+/−50) or about 250 (+/−20) adenosine nucleotides.

In some embodiments, the nucleic acid may comprise a poly(A) tail derived from a template DNA and may additionally comprise at least one additional poly(A) tail generated by enzymatic polyadenylation, e.g., as described in WO2016/091391.

In certain embodiments, the nucleic acid comprises at least one polyadenylation signal.

In various embodiments, the nucleic acid may comprise at least one poly(C) sequence.

The term “poly(C) sequence,” as used herein, is intended to be a sequence of cytosine nucleotides of up to about 200 cytosine nucleotides. In some embodiments, the poly(C) sequence comprises about 10 to about 200 cytosine nucleotides, about 10 to about 100 cytosine nucleotides, about 20 to about 70 cytosine nucleotides, about 20 to about 60 cytosine nucleotides, or about 10 to about 40 cytosine nucleotides. In some embodiments, the poly(C) sequence comprises about 30 cytosine nucleotides.

II. D. Chemical Modification

The mRNA disclosed herein may be modified or unmodified. In some embodiments, the mRNA may comprise at least one chemical modification. In some embodiments, the mRNA disclosed herein may contain one or more modifications that typically enhance RNA stability. Exemplary modifications can include backbone modifications, sugar modifications, or base modifications. In some embodiments, the disclosed mRNA may be synthesized from naturally occurring nucleotides and/or nucleotide analogues (modified nucleotides) including, but not limited to, purines (adenine (A) and guanine (G)) or pyrimidines (thymine (T), cytosine (C), and uracil (U)). In certain embodiments, the disclosed mRNA may be synthesized from modified nucleotide analogues or derivatives of purines and pyrimidines, such as, e.g., 1-methyl-adenine, 2-methyl-adenine, 2-methylthio-N-6-isopentenyl-adenine, N6-methyl-adenine, N6-isopentenyl-adenine, 2-thio-cytosine, 3-methyl-cytosine, 4-acetyl-cytosine, 5-methyl-cytosine, 2,6-diaminopurine, 1-methyl-guanine, 2-methyl-guanine, 2,2-dimethyl-guanine, 7-methyl-guanine, inosine, 1-methyl-inosine, pseudouracil (5-uracil), dihydro-uracil, 2-thio-uracil, 4-thio-uracil, 5-carboxymethylaminomethyl-2-thio-uracil, 5-(carboxyhydroxymethyl)-uracil, 5-fluoro-uracil, 5-bromo-uracil, 5-carboxymethylaminomethyl-uracil, 5-methyl-2-thio-uracil, 5-methyl-uracil, N-uracil-5-oxy acetic acid methyl ester, 5-methylaminomethyl-uracil, 5-methoxyaminomethyl-2-thio-uracil, 5′-methoxycarbonylmethyl-uracil, 5-methoxy-uracil, uracil-5-oxyacetic acid methyl ester, uracil-5-oxyacetic acid (v), 1-methyl-pseudouracil, queosine, β-D-mannosyl-queosine, phosphoramidates, phosphorothioates, peptide nucleotides, methylphosphonates, 7-deazaguanosine, 5-methylcytosine, and inosine.

In some embodiments, the disclosed mRNA may comprise at least one chemical modification including, but not limited to, pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine.

In some embodiments, the chemical modification is selected from the group consisting of pseudouridine, N1-methylpseudouridine, 5-methylcytosine, 5-methoxyuridine, and a combination thereof.

In some embodiments, the chemical modification comprises N1-methylpseudouridine.

In some embodiments, at least 20%, at least 30%, at least 40%, at least 50%, at least 60%, at least 70%, at least 80%, at least 85%, at least 90%, at least 95%, or 100% of the uracil nucleotides in the mRNA are chemically modified.

In some embodiments, at least 20%, at least 30%, at least 40%, at least 50%, at least 60%, at least 70%, at least 80%, at least 85%, at least 90%, at least 95%, or 100% of the uracil nucleotides in the ORF are chemically modified.

The preparation of such analogues is described, e.g., in U.S. Pat. Nos. 4,373,071, 4,401,796, 4,415,732, 4,458,066, 4,500,707, 4,668,777, 4,973,679, 5,047,524, 5,132,418, 5,153,319, 5,262,530, and 5,700,642.

II. E. mRNA Synthesis

The mRNAs disclosed herein may be synthesized according to any of a variety of methods. For example, mRNAs according to the present disclosure may be synthesized via in vitro transcription (IVT). Some methods for in vitro transcription are described, e.g., in Geall et al. (2013) Semin. Immunol. 25(2): 152-159; Brunelle et al. (2013) Methods Enzymol. 530:101-14. Briefly, IVT is typically performed with a linear or circular DNA template containing a promoter, a pool of ribonucleotide triphosphates, a buffer system that may include DTT and magnesium ions, an appropriate RNA polymerase (e.g., T3, T7, or SP6 RNA polymerase), DNase I, pyrophosphatase, and/or RNase inhibitor. The exact conditions may vary according to the specific application. The presence of these reagents is generally undesirable in a final mRNA product and these reagents can be considered impurities or contaminants which can be purified or removed to provide a clean and/or homogeneous mRNA that is suitable for therapeutic use. While mRNA provided from in vitro transcription reactions may be desirable in some embodiments, other sources of mRNA can be used according to the instant disclosure including wild-type mRNA produced from bacteria, fungi, plants, and/or animals.

### III. Processes for Making the Present LNP Vaccines

The present LNPs can be prepared by various techniques presently known in the art. For example, multilamellar vesicles (MLV) may be prepared according to conventional techniques, such as by depositing a selected lipid on the inside wall of a suitable container or vessel by dissolving the lipid in an appropriate solvent, and then evaporating the solvent to leave a thin film on the inside of the vessel or by spray drying. An aqueous phase may then be added to the vessel with a vortexing motion that results in the formation of MLVs. Unilamellar vesicles (ULV) can then be formed by homogenization, sonication or extrusion of the multilamellar vesicles. In addition, unilamellar vesicles can be formed by detergent removal techniques.

Various methods are described in US 2011/0244026, US 2016/0038432, US 2018/0153822, US 2018/0125989, and PCT/US2020/043223 (filed Jul. 23, 2020) and can be used to practice the present invention. One exemplary process entails encapsulating mRNA by mixing it with a mixture of lipids, without first pre-forming the lipids into lipid nanoparticles, as described in US 2016/0038432. Another exemplary process entails encapsulating mRNA by mixing pre-formed LNPs with mRNA, as described in US 2018/0153822.

In some embodiments, the process of preparing mRNA-loaded LNPs includes a step of heating one or more of the solutions to a temperature greater than ambient temperature, the one or more solutions being the solution comprising the pre-formed lipid nanoparticles, the solution comprising the mRNA and the mixed solution comprising the LNP-encapsulated mRNA. In some embodiments, the process includes the step of heating one or both of the mRNA solution and the pre-formed LNP solution, prior to the mixing step. In some embodiments, the process includes heating one or more of the solutions comprising the pre-formed LNPs, the solution comprising the mRNA and the solution comprising the LNP-encapsulated mRNA, during the mixing step. In some embodiments, the process includes the step of heating the LNP-encapsulated mRNA, after the mixing step. In some embodiments, the temperature to which one or more of the solutions is heated is or is greater than about 30° C., 37° C., 40° C., 45° C., 50° C., 55° C., 60° C., 65° C., or 70° C. In some embodiments, the temperature to which one or more of the solutions is heated ranges from about 25-70° C., about 30-70° C., about 35-70° C., about 40-70° C., about 45-70° C., about 50-70° C., or about 60-70° C. In some embodiments, the temperature is about 65° C.

Various methods may be used to prepare an mRNA solution suitable for the present invention. In some embodiments, mRNA may be directly dissolved in a buffer solution described herein. In some embodiments, an mRNA solution may be generated by mixing an mRNA stock solution with a buffer solution prior to mixing with a lipid solution for encapsulation. In some embodiments, an mRNA solution may be generated by mixing an mRNA stock solution with a buffer solution immediately before mixing with a lipid solution for encapsulation. In some embodiments, a suitable mRNA stock solution may contain mRNA in water or a buffer at a concentration at or greater than about 0.2 mg/ml, 0.4 mg/ml, 0.5 mg/ml, 0.6 mg/ml, 0.8 mg/ml, 1.0 mg/ml, 1.2 mg/ml, 1.4 mg/ml, 1.5 mg/ml, or 1.6 mg/ml, 2.0 mg/ml, 2.5 mg/ml, 3.0 mg/ml, 3.5 mg/ml, 4.0 mg/ml, 4.5 mg/ml, or 5.0 mg/ml.

In some embodiments, an mRNA stock solution is mixed with a buffer solution using a pump. Exemplary pumps include but are not limited to gear pumps, peristaltic pumps and centrifugal pumps. Typically, the buffer solution is mixed at a rate greater than that of the mRNA stock solution. For example, the buffer solution may be mixed at a rate at least 1×, 2×, 3×, 4×, 5×, 6×, 7×, 8×, 9×, 10×, 15×, or 20× greater than the rate of the mRNA stock solution. In some embodiments, a buffer solution is mixed at a flow rate ranging between about 100-6000 ml/minute (e.g., about 100-300 ml/minute, 300-600 ml/minute, 600-1200 ml/minute, 1200-2400 ml/minute, 2400-3600 ml/minute, 3600-4800 ml/minute, 4800-6000 ml/minute, or 60-420 ml/minute). In some embodiments, a buffer solution is mixed at a flow rate of, or greater than, about 60 ml/minute, 100 ml/minute, 140 ml/minute, 180 ml/minute, 220 ml/minute, 260 ml/minute, 300 ml/minute, 340 ml/minute, 380 ml/minute, 420 ml/minute, 480 ml/minute, 540 ml/minute, 600 ml/minute, 1200 ml/minute, 2400 ml/minute, 3600 ml/minute, 4800 ml/minute, or 6000 ml/minute.

In some embodiments, an mRNA stock solution is mixed at a flow rate ranging between about 10-600 ml/minute (e.g., about 5-50 ml/minute, about 10-30 ml/minute, about 30-60 ml/minute, about 60-120 ml/minute, about 120-240 ml/minute, about 240-360 ml/minute, about 360-480 ml/minute, or about 480-600 ml/minute). In some embodiments, an mRNA stock solution is mixed at a flow rate of or greater than about 5 ml/minute, 10 ml/minute, 15 ml/minute, 20 ml/minute, 25 ml/minute, 30 ml/minute, 35 ml/minute, 40 ml/minute, 45 ml/minute, 50 ml/minute, 60 ml/minute, 80 ml/minute, 100 ml/minute, 200 ml/minute, 300 ml/minute, 400 ml/minute, 500 ml/minute, or 600 ml/minute.

The process of incorporation of a desired mRNA into a lipid nanoparticle is referred to as “loading.” Exemplary methods are described in Lasic et al., FEBS Lett. (1992) 312:255-8. The LNP-incorporated nucleic acids may be completely or partially located in the interior space of the lipid nanoparticle, within the bilayer membrane of the lipid nanoparticle, or associated with the exterior surface of the lipid nanoparticle membrane. The incorporation of an mRNA into lipid nanoparticles is also referred to herein as “encapsulation” wherein the nucleic acid is entirely or substantially contained within the interior space of the lipid nanoparticle.

Suitable LNPs may be made in various sizes. In some embodiments, decreased size of lipid nanoparticles is associated with more efficient delivery of an mRNA. Selection of an appropriate LNP size may take into consideration the site of the target cell or tissue and to some extent the application for which the lipid nanoparticle is being made.

A variety of methods known in the art are available for sizing of a population of lipid nanoparticles. Preferred methods herein utilize Zetasizer Nano ZS (Malvern Panalytical) to measure LNP particle size. In one protocol, 10 μl of an LNP sample are mixed with 990 μl of 10% trehalose. This solution is loaded into a cuvette and then put into the Zetasizer machine. The z-average diameter (nm), or cumulants mean, is regarded as the average size for the LNPs in the sample. The Zetasizer machine can also be used to measure the polydispersity index (PDI) by using dynamic light scattering (DLS) and cumulant analysis of the autocorrelation function. Average LNP diameter may be reduced by sonication of formed LNP. Intermittent sonication cycles may be alternated with quasi-elastic light scattering (QELS) assessment to guide efficient lipid nanoparticle synthesis.

In some embodiments, the majority of purified LNPs, i.e., greater than about 50%, 55%, 60%, 65%, 70%, 75%, 80%, 85%, 90%, 95%, 96%, 97%, 98%, or 99% of the LNPs, have a size of about 70-150 nm (e.g., about 145 nm, about 140 nm, about 135 nm, about 130 nm, about 125 nm, about 120 nm, about 115 nm, about 110 nm, about 105 nm, about 100 nm, about 95 nm, about 90 nm, about 85 nm, or about 80 nm). In some embodiments, substantially all (e.g., greater than 80 or 90%) of the purified lipid nanoparticles have a size of about 70-150 nm (e.g., about 145 nm, about 140 nm, about 135 nm, about 130 nm, about 125 nm, about 120 nm, about 115 nm, about 110 nm, about 105 nm, about 100 nm, about 95 nm, about 90 nm, about 85 nm, or about 80 nm).

In some embodiments, the LNPs in the present composition have an average size of less than 150 nm, less than 120 nm, less than 100 nm, less than 90 nm, less than 80 nm, less than 70 nm, less than 60 nm, less than 50 nm, less than 30 nm, or less than 20 nm.

In some embodiments, greater than about 70%, 75%, 80%, 85%, 90%, 95%, 96%, 97%, 98%, 99% of the LNPs in the present composition have a size ranging from about 40-90 nm (e.g., about 45-85 nm, about 50-80 nm, about 55-75 nm, about 60-70 nm) or about 50-70 nm (e.g., 55-65 nm) are particular suitable for pulmonary delivery via nebulization.

In some embodiments, the dispersity, or measure of heterogeneity in size of molecules (PDI), of LNPs in a pharmaceutical composition provided by the present invention is less than about 0.5. In some embodiments, an LNP has a PDI of less than about 0.5, less than about 0.4, less than about 0.3, less than about 0.28, less than about 0.25, less than about 0.23, less than about 0.20, less than about 0.18, less than about 0.16, less than about 0.14, less than about 0.12, less than about 0.10, or less than about 0.08. The PDI may be measured by a Zetasizer machine as described above.

In some embodiments, greater than about 75%, 80%, 85%, 90%, 95%, 96%, 97%, 98%, or 99% of the purified LNPs in a pharmaceutical composition provided herein encapsulate an mRNA within each individual particle. In some embodiments, substantially all (e.g., greater than 80% or 90%) of the purified lipid nanoparticles in a pharmaceutical composition encapsulate an mRNA within each individual particle. In some embodiments, a lipid nanoparticle has an encapsulation efficiency of between 50% and 99%; or greater than about 60, 65, 70, 75, 80, 85, 90, 92, 95, 98, or 99%. Typically, lipid nanoparticles for use herein have an encapsulation efficiency of at least 90% (e.g., at least 91, 92, 93, 94, or 95%).

In some embodiments, an LNP has a N/P ratio of between 1 and 10. In some embodiments, a lipid nanoparticle has a N/P ratio above 1, about 1, about 2, about 3, about 4, about 5, about 6, about 7, or about 8. In further embodiments, a typical LNP herein has an N/P ratio of 4.

In some embodiments, a pharmaceutical composition according to the present invention contains at least about 0.5 μg, 1 μg, 5 μg, 10 μg, 100 μg, 500 μg, or 1000 μg of encapsulated mRNA. In some embodiments, a pharmaceutical composition contains about 0.1 μg to 1000 μg, at least about 0.5 μg, at least about 0.8 μg, at least about 1 μg, at least about 5 μg, at least about 8 μg, at least about 10 μg, at least about 50 μg, at least about 100 μg, at least about 500 μg, or at least about 1000 μg of encapsulated mRNA.

In some embodiments, mRNA can be made by chemical synthesis or by in vitro transcription (IVT) of a DNA template. An exemplary process for making and purifying mRNA is described in Example 1. In this process, in an IVT process, a cDNA template is used to produce an mRNA transcript and the DNA template is degraded by a DNase. The transcript is purified by depth filtration and tangential flow filtration (TFF). The purified transcript is further modified by adding a cap and a tail, and the modified RNA is purified again by depth filtration and TFF.

The mRNA is then prepared in an aqueous buffer and mixed with an amphiphilic solution containing the lipid components of the LNPs. An amphiphilic solution for dissolving the four lipid components of the LNPs may be an alcohol solution. In some embodiments, the alcohol is ethanol. The aqueous buffer may be, for example, a citrate, phosphate, acetate, or succinate buffer and may have a pH of about 3.0-7.0, e.g., about 3.5, about 4.0, about 4.5, about 5.0, about 5.5, about 6.0, or about 6.5. The buffer may contain other components such as a salt (e.g., sodium, potassium, and/or calcium salts). In particular embodiments, the aqueous buffer has 1 mM citrate, 150 mM NaCl, pH 4.5.

An exemplary, nonlimiting process for making an mRNA-LNP composition is described in Example 1. The process involves mixing of a buffered mRNA solution with a solution of lipids in ethanol in a controlled homogeneous manner, where the ratio of lipids:mRNA is maintained throughout the mixing process. In this illustrative example, the mRNA is presented in an aqueous buffer containing citric acid monohydrate, tri-sodium citrate dihydrate, and sodium chloride. The mRNA solution is added to the solution (1 mM citrate buffer, 150 mM NaCl, pH 4.5). The lipid mixture of four lipids (e.g., a cationic lipid, a PEGylated lipid, a cholesterol-based lipid, and a helper lipid) is dissolved in ethanol. The aqueous mRNA solution and the ethanol lipid solution are mixed at a volume ratio of 4:1 in a “T” mixer with a near “pulseless” pump system. The resultant mixture is then subjected for downstream purification and buffer exchange. The buffer exchange may be achieved using dialysis cassettes or a TFF system. TFF may be used to concentrate and buffer-exchange the resulting nascent LNP immediately after formation via the T-mix process. The diafiltration process is a continuous operation, keeping the volume constant by adding appropriate buffer at the same rate as the permeate flow.

IV. Packaging and Use of the mRNA-LNP Vaccines

The mRNA-LNP vaccines can be packaged for parenteral (e.g., intramuscular, intradermal or subcutaneous) administration or nasopharyngeal (e.g., intranasal) administration. The vaccine compositions may be in the form of an extemporaneous formulation, where the LNP composition is lyophilized and reconstituted with a physiological buffer (e.g., PBS) just before use. The vaccine compositions also may be shipped and provided in the form of an aqueous solution or a frozen aqueous solution and can be directly administered to subjects without reconstitution (after thawing, if previously frozen).

Accordingly, the present disclosure provides an article of manufacture, such as a kit, that provides the mRNA-LNP vaccine in a single container, or provides the mRNA-LNP vaccine in one container and a physiological buffer for reconstitution in another container. The container(s) may contain a single-use dosage or multi-use dosage. The containers may be pre-treated glass vials or ampules. The article of manufacture may include instructions for use as well.

In certain embodiments, the mRNA-LNP vaccine is provided for use in intramuscular (IM) injection. The vaccine can be injected to a subject at, e.g., his/her deltoid muscle in the upper arm. In some embodiments, the vaccine is provided in a pre-filled syringe or injector (e.g., single-chambered or multi-chambered). In some embodiments, the vaccine is provided for use in inhalation and is provided in a pre-filled pump, aerosolizer, or inhaler.

The mRNA-LNP vaccines can be administered to subjects in need thereof in a prophylactically effective amount, i.e., an amount that provides sufficient immune protection against a target pathogen for a sufficient amount of time (e.g., one year, two years, five years, ten years, or life-time). Sufficient immune protection may be, for example, prevention or alleviation of symptoms associated with infections by the pathogen. In some embodiments, multiple doses (e.g., two doses) of the vaccine are injected to subjects in need thereof to achieve the desired prophylactic effects. The doses (e.g., prime and booster doses) may be separated by an interval of e.g., 1 week, 2 weeks, 3 weeks, 4 weeks, one month, two months, three months, four months, five months, six months, one year, two years, five years, or ten years.

In some embodiments, a single dose of the mRNA-LNP vaccine contains 1-50 μg of mRNA (e.g., monovalent or multivalent). For example, a single dose may contain about 2.5 μg, about 5 μg, about 7.5 μg, about 10 μg, about 12.5 μg, or about 15 μg of the mRNA for intramuscular (IM) injection. In further embodiments, a multi-valent single dose of an LNP vaccine contains multiple (e.g., 2, 3, or 4) kinds of LNPs, each for a different antigen, and each kind of LNP has an mRNA amount of, e.g., 2.5 μg, about 5 μg, about 7.5 μg, about 10 μg, about 12.5 μg, or about 15 μg.

In another aspect, the present invention provides methods of immunizing a subject against one or more influenza viruses in a subject. The present invention further provides methods of eliciting an immune response against one or more influenza viruses in a subject. In some embodiments, the present methods comprise administering to the subject an effective amount of a composition described herein to a subject.

In various embodiments, the methods of immunizing provided herein elicit a broadly protective immune response against multiple epitopes within one or more influenza viruses. In various embodiments, the methods of immunizing provided herein elicit a broadly neutralizing immune response against one or more influenza viruses. In some embodiments, the immune response comprises an antibody response. Accordingly, in various embodiments, the composition described herein can offer broad cross-protection against different types of influenza viruses. In some embodiments, the composition offers cross-protection against avian, swine, seasonal, and/or pandemic influenza viruses. In some embodiments, the composition offers cross-protection against one or more influenza A, B, or C subtypes. In some embodiments, the composition offers cross-protection against multiple strains of influenza A H1-subtype viruses (e.g., H1N1), influenza A H3-subtype viruses (e.g., H3N2), influenza A H5-subtype viruses (e.g., H5N1), and/or influenza B viruses (e.g., Yamagata lineage, Victoria lineage).

In some embodiments, the methods of the invention are capable of eliciting an improved immune response against one or more seasonal influenza strains. Exemplary seasonal strains include, without limitation, A/Puerto Rico/8/1934, A/Fort Monmouth/l/1947, A/Chile/i/1983, A/Texas/36/1991, A/Singapore/6/1986, A/Beijing/32/1992, A/New Caledonia/20/1999, A/Solomon Islands/03/2006, A/Brisbane/59/2007, A(H3N2) virus antigenically like the cell-propagated prototype virus A/Victoria/361/2011, A/Beijing/262/95 (H1N1)-like virus, A/Brisbane/02/2018 (H1N1)pdm09-like virus, A/Brisbane/10/2007 (H3N2)-like virus, A/California/7/2004 (H3N2)-like virus, A/California/7/2009 (H1N1)-like virus, A/California/7/2009 (H1N1)pdm09-like virus, A/Cambodia/e0826360/2020 (H3N2)-like virus, A/Fujian/411/2002 (H3N2) -like virus, A/Fujian/411/2002 (H3N2)-like virus, A/Guangdong-Maonan/SWL1536/2019 (H1N1)pdm09-like virus-like virus, A/Hawaii/70/2019 (H1N1)pdm09-like virus-like virus, A/Hong Kong/2671/2019 (H3N2)-like virus, A/Hong Kong/45/2019 (H3N2)-like virus, A/Hong Kong/4801/2014 (H3N2)-like virus, A/Kansas/14/2017 (H3N2)-like virus, A/Michigan/45/2015 (H1N1)pdm09-like virus, A/Moscow/10/99 (H3N2)-like virus, A/New Caledonia/20/99 (H1N1)-like virus, A/Perth/16/2009 (H3N2)-like virus, A/Singapore/INFIMH-16-0019/2016 (H3N2)-like virus, A/Solomon Islands/3/2006 (H1N1)-like virus, A/South Australia/34/2019 (H3N2)-like virus, A/Switzerland/8060/2017 (H3N2)-like virus, A/Switzerland/9715293/2013 (H3N2)-like virus, A/Sydney/5/97 (H3N2)-like virus, A/Texas/50/2012 (H3N2)-like virus, A/Victoria/2570/2019 (H1N1)pdm09-like virus, A/Victoria/2570/2019 (H1N1)pdm09-like virus-like virus, A/Victoria/361/2011 (H3N2)-like virus, A/Wellington/1/2004 (H3N2)-like virus, A/Wisconsin/588/2019 (H1N1)pdm09-like virus, A/Wisconsin/588/2019 (H1N1)pdm09-like virus-like virus, A/Wisconsin/67/2005 (H3N2)-like virus, B/Beijing/184/93-like virus, B/Brisbane/60/2008-like virus, B/Colorado/06/2017-like virus (B/Victoria/2/87 lineage), B/Florida/4/2006-like virus, B/Hong Kong/330/2001-like virus, B/Malaysia/2506/2004-like virus, B/Massachusetts/2/2012-like virus, B/Phuket/3073/2013 (B/Yamagata lineage)-like virus, B/Phuket/3073/2013-like virus, B/Phuket/3073/2013-like virus (B/Yamagata/16/88 lineage), B/Shangdong/7/97-like virus, B/Shanghai/361/2002-like virus, B/Sichuan/379/99-like virus, B/Washington/02/2019 (B/Victoria lineage)-like virus, B/Washington/02/2019-like (B/Victoria lineage) virus, and B/Wisconsin/1/2010-like virus. In some embodiments, the methods of the invention are capable of eliciting an improved immune response against one or more pandemic influenza strains. Exemplary pandemic strains include, without limitation, A/California/07/2009, A/California/04/2009, A/Belgium/145/2009, A/South Carolina/01/1918, and A/New Jersey/1976. Pandemic subtypes include, in particular, the H1N1, H5N1, H2N2, H3N2, H9N2, H7N7, H7N3, H7N9 and H10N7 subtypes. In some embodiments, the methods of the invention are capable of eliciting an improved immune response against one or more swine influenza strains. Exemplary swine strains include, without limitation, A/New Jersey/1976 isolates and A/California/07/2009 In some embodiments, the methods of the invention are capable of eliciting an improved immune response against one or more avian influenza strains. Exemplary avian strains include, without limitation, H5N1, H7N3, H7N7, H7N9, and H9N2. Additional influenza pandemic, seasonal, avian and/or swine strains are known in the art.

In some embodiments, the present invention provides methods of preventing or treating influenza infections by administering the composition of the invention to a subject in need thereof. In some embodiments, the subject is suffering from or susceptible to an influenza infection. In some embodiments, a subject is considered to be suffering from an influenza infection if the subject is displaying one or more symptoms commonly associated with influenza infection. In some embodiments, the subject is known or believed to have been exposed to the influenza virus. In some embodiments, a subject is considered to be susceptible to an influenza infection if the subject is known or believed to have been exposed to the influenza virus. In some embodiments, a subject is known or believed to have been exposed to the influenza virus if the subject has been in contact with other individuals known or suspected to have been infected with the influenza virus and/or if the subject is or has been present in a location in which influenza infection is known or thought to be prevalent.

In various embodiments, the composition as described herein may be administered prior to or after development of one or more symptoms of influenza infection. In some embodiments, the composition is administered as a prophylactic. In such embodiments, the methods of the invention are effective in preventing or protecting a subject from influenza virus infection. In some embodiments, the composition of the present invention is used as a component of a seasonal and/or pandemic influenza vaccine or as part of an influenza vaccination regimen intended to confer long-lasting (multi-season) protection. In some embodiments, the composition of the presenting invention is used to treat the symptoms of influenza infection.

In some embodiments, the subject is a non-human mammal. In some embodiments, the subject is a farm animal or a pet (e.g., a dog, a cat, a sheep, cattle, and/or a pig). In some embodiments, the subject is a non-human primate. In some embodiments, the subject is an avian (e.g., a chicken).

In some embodiments, the subject is a human. In certain embodiments, the subject is an adult, an adolescent, or an infant. In some embodiments, the human subject is younger than 6 months of age. In some embodiments, the human subject is 6 months of age or older, is 6 months through 35 months of age, is 36 months through 8 years of age, or 9 years of age or older. In some embodiments, the human subject is an elderly aged 55 years or older, such as 60 year of age or older, or 65 years of age or older. Also contemplated by the present invention are the administration of the composition and/or performance of the methods of treatment in-utero.

Unless otherwise defined herein, scientific and technical terms used in connection with the present invention shall have the meanings that are commonly understood by those of ordinary skill in the art. Exemplary methods and materials are described below, although methods and materials similar or equivalent to those described herein can also be used in the practice or testing of the present invention. In case of conflict, the present specification, including definitions, will control. Generally, nomenclature used in connection with, and techniques of, cell and tissue culture, molecular biology, virology, immunology, microbiology, genetics, analytical chemistry, synthetic organic chemistry, medicinal and pharmaceutical chemistry, and protein and nucleic acid chemistry and hybridization described herein are those well-known and commonly used in the art. Enzymatic reactions and purification techniques are performed according to manufacturer's specifications, as commonly accomplished in the art or as described herein. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety. Although a number of documents are cited herein, this citation does not constitute an admission that any of these documents forms part of the common general knowledge in the art. As used herein, the term “approximately” or “about” as applied to one or more values of interest refers to a value that is similar to a stated reference value. In certain embodiments, the term refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context.

### V. Vectors

In one aspect, disclosed herein are vectors comprising the mRNA compositions disclosed herein. The RNA sequences encoding a protein of interest (e.g., mRNA encoding an influenza protein) can be cloned into a number of types of vectors. For example, the nucleic acids can be cloned into a vector including, but not limited to, a plasmid, a phagemid, a phage derivative, an animal virus, and a cosmid. Vectors of particular interest can include expression vectors, replication vectors, probe generation vectors, sequencing vectors, and vectors optimized for in vitro transcription.

In certain embodiments, the vector can be used to express mRNA in a host cell. In various embodiments, the vector can be used as a template for IVT. The construction of optimally translated IVT mRNA suitable for therapeutic use is disclosed in detail in Sahin, et al. (2014). Nat. Rev. Drug Discov. 13, 759-780; Weissman (2015). Expert Rev. Vaccines 14, 265-281.

In some embodiments, the vectors disclosed herein can comprise at least the following, from 5′ to 3′: an RNA polymerase promoter; a polynucleotide sequence encoding a 5′ UTR; a polynucleotide sequence encoding an ORF; a polynucleotide sequence encoding a 3′ UTR; and a polynucleotide sequence encoding at least one RNA aptamer. In some embodiments, the vectors disclosed herein may comprise a polynucleotide sequence encoding a poly(A) sequence and/or a polyadenylation signal.

A variety of RNA polymerase promoters are known. In some embodiments, the promoter can be a T7 RNA polymerase promoter. Other useful promoters can include, but are not limited to, T3 and SP6 RNA polymerase promoters. Consensus nucleotide sequences for T7, T3, and SP6 promoters are known.

Also disclosed herein are host cells (e.g., mammalian cells, e.g., human cells) comprising the vectors or RNA compositions disclosed herein.

Polynucleotides can be introduced into target cells using any of a number of different methods, for instance, commercially available methods which include, but are not limited to, electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendorf, Hamburg, Germany), cationic liposome mediated transfection using lipofection, polymer encapsulation, peptide mediated transfection, biolistic particle delivery systems such as “gene guns” (see, for example, Nishikawa, et al. (2001). Hum Gene Ther. 12(8):861-70, or the TransIT-RNA transfection Kit (Mirus, Madison, Wis.).

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

Regardless of the method used to introduce exogenous nucleic acids into a host cell or otherwise expose a cell to the inhibitor of the present disclosure, in order to confirm the presence of the mRNA sequence in the host cell a variety of assays may be performed.

### VI. Self-Replicating RNA and Trans-Replicating RNA

Self-Replicating RNA:

In one aspect, disclosed herein are self-replicating RNAs encoding an influenza protein.

Self-replicating RNA can be produced by using replication elements derived from, e.g., alphaviruses, and substituting the structural viral proteins with a nucleotide sequence encoding a protein of interest (e.g., influenza protein). A self-replicating RNA is typically a positive-strand molecule which can be directly translated after delivery to a cell, and this translation provides an RNA-dependent RNA polymerase which then produces both antisense and sense transcripts from the delivered RNA. Thus, the delivered RNA leads to the production of multiple daughter RNAs. These daughter RNAs, as well as collinear subgenomic transcripts, may be translated themselves to provide in situ expression of an encoded antigen (i.e., an influenza protein antigen), or may be transcribed to provide further transcripts with the same sense as the delivered RNA which are translated to provide in situ expression of the antigen. The overall result of this sequence of transcriptions is a large amplification in the number of the introduced replicon RNAs and so the encoded antigen becomes a major polypeptide product of the cells.

One suitable system for achieving self-replication in this manner is to use an alphavirus-based replicon. These replicons are positive stranded (positive sense-stranded) RNAs which lead to translation of a replicase (or replicase-transcriptase) after delivery to a cell. The replicase is translated as a polyprotein which auto-cleaves to provide a replication complex which creates genomic-strand copies of the positive-strand delivered RNA. These negative (−)-stranded transcripts can themselves be transcribed to give further copies of the positive-stranded parent RNA and also to give a subgenomic transcript which encodes the antigen. Translation of the subgenomic transcript thus leads to in situ expression of the antigen by the infected cell. Suitable alphavirus replicons can use a replicase from a Sindbis virus, a Semliki forest virus, an eastern equine encephalitis virus, a Venezuelan equine encephalitis virus, etc. Mutant or wild-type virus sequences can be used, e.g., the attenuated TC83 mutant of VEEV has been used in replicons, see the following reference: WO2005/113782, incorporated herein by reference.

In one embodiment, each self-replicating RNA described herein encodes (i) an RNA-dependent RNA polymerase which can transcribe RNA from the self-replicating RNA molecule and (ii) an influenza protein antigen. The polymerase can be an alphavirus replicase, e.g., comprising one or more of alphavirus proteins nsP1, nsP2, nsP3, and nsP4. Whereas natural alphavirus genomes encode structural virion proteins in addition to the non-structural replicase polyprotein, in certain embodiments, the self-replicating RNA molecules do not encode alphavirus structural proteins. Thus, the self-replicating RNA can lead to the production of genomic RNA copies of itself in a cell, but not to the production of RNA-containing virions. The inability to produce these virions means that, unlike a wild-type alphavirus, the self-replicating RNA molecule cannot perpetuate itself in infectious form. The alphavirus structural proteins which are necessary for perpetuation in wild-type viruses are absent from self-replicating RNAs of the present disclosure and their place is taken by gene(s) encoding the immunogen of interest, such that the subgenomic transcript encodes the immunogen rather than the structural alphavirus virion proteins. Self-replicating RNA are described in further detail in WO2011005799, incorporated herein by reference.

Trans-Replicating RNA:

In one aspect, disclosed herein are trans-replicating RNAs encoding an influenza protein.

Trans-replicating RNA possess similar elements as the self-replicating RNA described above. However, with trans replicating RNA, two separate RNA molecules are used. A first RNA molecule encodes for the RNA replicase described above (e.g., the alphavirus replicase) and a second RNA molecule encodes for the protein of interest (e.g., an influenza protein antigen). The RNA replicase may replicate one or both of the first and second RNA molecule, thereby greatly increasing the copy number of RNA molecules encoding the protein of interest. Trans replicating RNA are described in further detail in WO2017162265, incorporated herein by reference.

VII. Pharmaceutical Compositions

RNA purified according to this disclosure can be useful as a component in pharmaceutical compositions, for example, for use as a vaccine. These compositions will typically include RNA and a pharmaceutically acceptable carrier. A pharmaceutical composition of the present disclosure can also include one or more additional components such as small molecule immunopotentiators (e.g., TLR agonists). A pharmaceutical composition of the present disclosure can also include a delivery system for the RNA, such as a liposome, an oil-in-water emulsion, or a microparticle. In some embodiments, the pharmaceutical composition comprises a lipid nanoparticle (LNP). In certain embodiments, the composition comprises an antigen-encoding nucleic acid molecule encapsulated within an LNP.

### VIII. Methods of Vaccination

The influenza vaccine disclosed herein may be administered to a subject to induce an immune response directed against one or more influenza protein, wherein an anti-antigen antibody titer in the subject is increased following vaccination relative to an anti-antigen antibody titer in a subject that is not vaccinated with the influenza vaccine disclosed herein, or relative to an alternative vaccine against influenza. An “anti-antigen antibody” is a serum antibody that binds specifically to the antigen.

In one aspect, the disclosure provides a method of eliciting an immune response to influenza or protecting a subject against influenza infection comprising administering the influenza vaccine described herein to a subject. The disclosure also provides an influenza vaccine described herein for use in eliciting an immune response to influenza or in protecting a subject against influenza infection. The disclosure also provides an influenza mRNA described herein for use in the manufacture of a vaccine for eliciting an immune response to influenza or for protecting a subject against influenza infection.

In order that this invention may be better understood, the following examples are set forth. These examples are for purposes of illustration only and are not to be construed as limiting the scope of the invention in any manner.

### EXAMPLES

### Example 1: Optimization of LNP Formulations

This Example describes a study in which a series of LNP formulations for mRNA vaccines were prepared from combinatorial libraries of various components. Rationally designed novel cationic lipids were synthesized. Altogether, more than 150 lipids and more than 430 formulations were tested. Human Erythropoietin (hEPO) mRNA was used as a test mRNA. In the lead formulations described below, the mRNA was formulated into LNP using combinations of the cationic lipids and the three other lipids—helper lipids; cholesterol-based lipids; and PEGylated lipids—in various permutations of combinations.

The LNP formulations consisted of four lipid components—ionizable lipid, helper lipid DOPE, cholesterol, and PEGylated lipid DMG-PEG-2K. The PEGylated lipid molar fraction was held constant at 1.5%, while the ionizable lipid and the different helper lipids and their molar ratios were evaluated to identify the optimized ratios based on the hEPO screening studies.

Citrate buffer (1 mM citrate, 150 mM NaCl, pH 4.5) was used in the preparation of LNP formulation. mRNA solution added to the citrate buffer was mixed with the lipids in ethanol solution during the formulation process. The pH and the concentration of the buffer were selected to achieve the high rate of mRNA encapsulation in the LNP formulation.

The LNP formulation process included mixing the lipid ethanol solution and the mRNA citrate solution in a ‘T’ mixer using a pump system. The resultant solution was then subjected to buffer exchange using TFF/dialysis tubes. The concentration of the final formulation in 10% (w/v) trehalose was adjusted based on dosing needs.

Mouse in vivo expression of hEPO protein was used as a surrogate to measure the potency of the LNPs to delivery mRNA in vivo. In this study, a single dose of hEPO mRNA (0.1 μg) formulated in LNPs derived from various combinations of the components was injected into mice intramuscularly (IM). Serum collected at 6 hours and 24 hours after administration was tested for hEPO levels using ELISA. MC3 formulation, an industry benchmark, was used a reference for the calculation of fold-increase in hEPO expression (Angew, Chem Int Ed. (2012) 51:8529-33).

The level of hEPO expression seen for each LNP formulation indicated the formulation's ability to deliver mRNA into cells. The initial formulations included 2-dioleoyl-sn-glycero-3-phosphoethanolamine (DOPE; helper lipid), DMG-PEG2000, and cholesterol at the molar ratio of cationic lipid:DMG-PEG2000:cholesterol:DOPE at 40:1.5:28.5:30. These formulations were found to have robust potency when compared to MC3 formulations.

Further formulations were tested. Optimized formulations Lipid A LNP and Lipid B LNP are shown in Table 1. The mRNA in these formulations can be modified or unmodified and may encode an antigen derived from influenza.

TABLE 1
Composition of Exemplary LNP Formulations

| Components | | Function | Description |
|---|---|---|---|
| mRNA | | Active substance | mRNA Construct |
| lipid | Cationic Lipid OF-02 | Delivery | Ionizable lipid, facilitates |
| nanoparticle | (A) or cKK-E10 (B) | | mRNA encapsulation |
| (LNP) | DOPE | | Zwitterionic lipid, enhances uptake |
| | | | and release of drug pay load |
| | Cholesterol | | Provides stability to lipid bilayer |
| | DMG-PEG-2K | | Provides control and |
| | | | stability to the lipid bilayer |

| | | |
|---|---|---|
| Trehalose | Excipient | Cryoprotectant |
| Water for Injection (WFI) | Diluent | N/A |

In Table 1, the final dosing for a human vaccine would be dilution of the above final bulk product in phosphate-buffered saline (PBS) based on the intended single human dose. The WFI amount is calculated based upon nominal of final drug product. Trehalose content in the formulation corresponds to 10% (100 mg/mL) trehalose dihydrate, converted to an anhydrous basis using the ratio of the molecular weight values of anhydrous trehalose and trehalose dihydrate.

The molar ratios of lipid components in two optimized formulations—Lipid A and Lipid B LNP formulations—are shown in Table 2 (CL: cationic lipid).

TABLE 2
Molar Ratios of Lipid Components in Exemplary LNPs

| | | Molar Ratios of CL: |
|---|---|---|
| | | DMG-PEG2000: |
| CL | LNP Code | Cholesterol: DOPE |
| OF-02 | Lipid A | 40:1.5:28.5:30 |
| cKK-E10 | Lipid B | 40:1.5:28.5:30 |

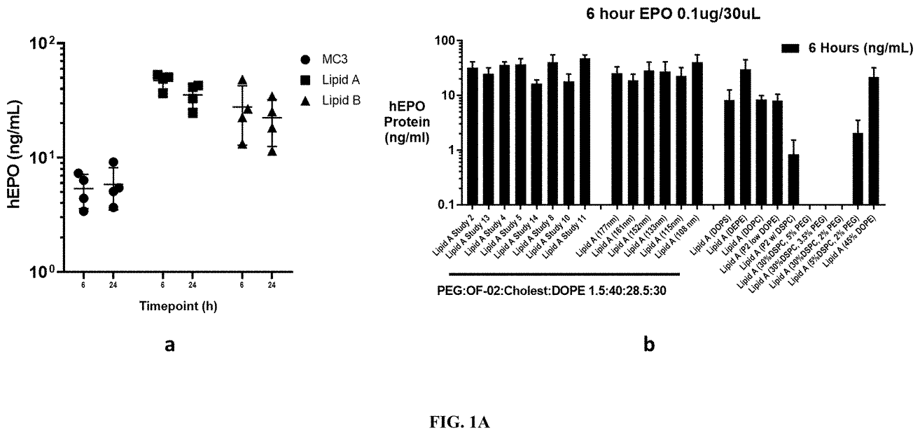

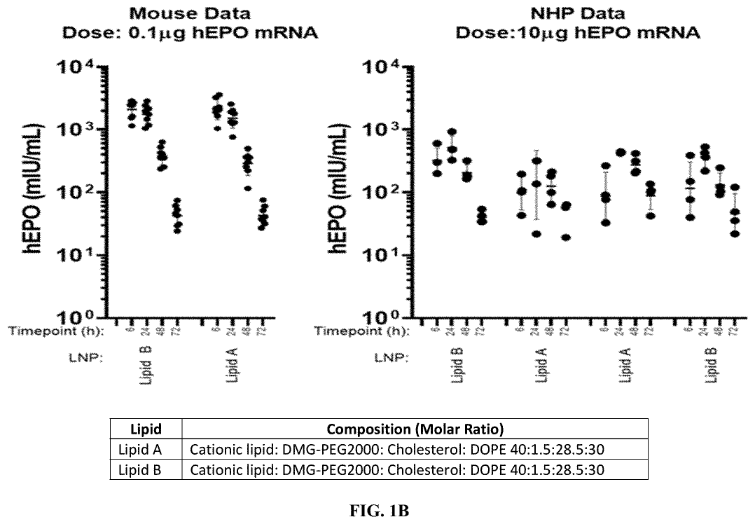

As shown in Table 3 and FIG. 1A, the fold increase of hEPO expression for Lipid A and Lipid B compared to MC3 indicates the superiority of these LNPs over MC3 for the delivery of mRNA. In the table below, “P2” means PEG2000; “Times MC3” means the fold of increase over MC3; and “Std Dev” means standard deviation.

TABLE 3
In vivo Delivery of hEPO mRNA in Mice

| Study # | Cationic lipid | Formulation Composition | Times MC3 | Std Dev |
|---|---|---|---|---|
| 1 | OF-02 | Cationic lipid: DMG-PEG2000: | 1.74 | 0.97 |
| | (P2 low DOPE) | cholesterol: DOPE 40:3:27:30 | | |
| | OF-02 | Cationic lipid: DMG-PEG2000: | 0.18 | 0.17 |
| | (P2 w/DSPC) | cholesterol: DSPC 50:1.5:38.5:10 | | |
| 2 | OF-02 | Cationic lipid: DMG-PEG2000: | 5.04 | 1.79 |
| | | cholesterol: DOPE 40:1.5:28.5:30 | | |
| 3 | OF-02 | Cationic lipid: DMG-PEG2000: | 7.35 | 3.90 |
| | (high DOPE) | cholesterol: DOPE 40:1.5:13.5:45 | | |
| 4 | OF-02 | Cationic lipid: DMG-PEG2000: | 16.19 | 7.86 |
| | | cholesterol: DOPE 40:1.5:28.5:30 | | |
| 5 | OF-02 | Cationic lipid: DMG-PEG2000: | 12.13 | 6.56 |
| | | cholesterol: DOPE 40:1.5:28.5:30 | | |
| 6 | cKK-E10 | Cationic lipid: DMG-PEG2000: | 5.41 | 3.46 |
| | | cholesterol: DOPE 40:1.5:28.5:30 | | |
| 7 | cKK-E10 | Cationic lipid: DMG-PEG2000: | 5.77 | 2.09 |
| | (DEPE) | cholesterol: DEPE 40:1.5:28.5:30 | | |
| 8 | OF-02 | Cationic lipid: DMG-PEG2000: | 6.59 | 2.50 |
| | (177 nm) | cholesterol: DOPE 40:1.5:28.5:30 | | |
| | OF-02 | Cationic lipid: DMG-PEG2000: | 4.94 | 1.75 |
| | (161 nm) | cholesterol: DOPE 40:1.5:28.5:30 | | |
| | OF-02 | Cationic lipid: DMG-PEG2000: | 7.40 | 3.54 |
| | (153 nm) | cholesterol: DOPE 40:1.5:28.5:30 | | |
| | OF-02 | Cationic lipid: DMG-PEG2000: | 7.15 | 3.86 |
| | (133 nm) | cholesterol: DOPE 40:1.5:28.5:30 | | |
| | OF-02 | Cationic lipid: DMG-PEG2000: | 5.91 | 2.79 |
| | (115 nm) | cholesterol: DOPE 40:1.5:28.5:30 | | |
| | OF-02 | Cationic lipid: DMG-PEG2000: | 10.54 | 4.38 |
| | (118 nm) | cholesterol: DOPE 40:1.5:28.5:30 | | |
| 9 | OF-02 | Cationic lipid: DMG-PEG2000: | 0.00 | 0.00 |
| | (DSPC) | cholesterol: DSPC 40:5:25:30 | | |
| | OF-02 | Cationic lipid: DMG-PEG2000: | 0.00 | 0.00 |
| | (DSPC) | cholesterol: DSPC 40:3.5:26.5:30 | | |
| | OF-02 | Cationic lipid: DMG-PEG2000: | 0.00 | 0.00 |
| | (DSPC) | cholesterol: DSPC 40:2:28:30 | | |
| | OF-02 | Cationic lipid: DMG-PEG2000: | 0.99 | 0.70 |
| | (DSPC) | cholesterol: DSPC 40:2:53:5 | | |
| 10 | OF-02 | Cationic lipid: DMG-PEG2000: | 3.26 | 1.97 |
| | (DOPS) | cholesterol: DOPS 40:1.5:28.5:30 | | |
| | OF-02 | Cationic lipid: DMG-PEG2000: | 11.83 | 6.89 |
| | (DEPE) | cholesterol: DEPE 40:1.5:28.5:30 | | |
| | OF-02 | Cationic lipid: DMG-PEG2000: | 3.32 | 1.20 |
| | (DOPC) | cholesterol: DOPC 40:1.5:28.5:30 | | |
| | OF-02 | Cationic lipid: DMG-PEG2000: | 7.14 | 3.37 |
| | | cholesterol: DOPE 40:1.5:28.5:30 | | |
| 11 | cKK-E10 | Cationic lipid: DMG-PEG2000: | 5.58 | 2.01 |
| | | cholesterol: DOPE 40:1.5:28.5:30 | | |
| | OF-02 | Cationic lipid: DMG-PEG2000: | 8.81 | 3.22 |
| | (PD lot) | cholesterol: DOPE 40:1.5:28.5:30 | | |
| | cKK-E10 | Cationic lipid: DMG-PEG2000: | 5.16 | 3.25 |
| | | cholesterol: DOPE 40:1.5:28.5:30 | | |

FIG. 1B shows hEPO expression in mice and non-human primates (NHPs) using LNPs Lipid A and Lipid B. A single dose of hEPO mRNA (0.1 μg for mice and 10 μg for NHPs) formulated with Lipid A or Lipid B was injected intramuscularly. Serum hEPO levels were quantified at 6, 24, 48, and 72 hours after administration using ELISA. The data show prolonged hEPO protein expression in vivo even beyond 4 days in mice and NHPs.

One of the key process parameters identified during optimization was the flow rate during initial mixing step. Formulations with different final LNP sizes (ranging from 108-177 nm) were prepared by changing these flow rates during mixing, allowing additional control on process and product attributes. The higher the flow rate, the smaller the particle size. When the flow rate reached 375 ml/min, producing an average LNP size of 108 nM, there was a markedly increased potency. The impact of size on potency of LNP was noted as a measure of fold increase in hEPO expression over MC3 as Table 4.

TABLE 4
LNP Size Optimization

| Formulation | Total Flow rate | Size | | Encapsulation | Cationic | Times |
|---|---|---|---|---|---|---|
| Lot# | (ml/min) | (nm) | PDI | (%) | Lipid | MC3 |
| 1 | 250 | 108 | 0.077 | 99 | MC3 | 1.00 |
| 2 | 62.5 | 177 | 0.086 | 94 | OF-02 | 6.59 |
| 3 | 75 | 161 | 0.075 | 95 | OF-02 | 4.94 |
| 2-88 | 87.5 | 152 | 0.116 | 97 | OF-02 | 7.40 |
| 2-89 | 125 | 133 | 0.089 | 97 | OF-02 | 7.15 |
| 2-90 | 250 | 115 | 0.076 | 98 | OF-02 | 5.91 |
| 2-91 | 375 | 108 | 0.042 | 98 | OF-02 | 10.54 |

*PDI: polydispersity index.

The above screening data show that helper lipid DOPE was effective in promoting protein expression. The data also led to determination of the promising molar composition of the four lipids (OF-02 or cKK-E10:DMG-PEG-2K:cholesterol:DOPE=40:1.5:28.5:30). LNP formulations in 10% trehalose were characterized for all parameters including particle size, PDI, mRNA encapsulation, and mRNA integrity. All the tested batches showed the desired characteristics and stability in freeze/thaw cycling. The long-term stability of the formulation at −80° C. in 10% (w/v) trehalose was assessed. Lipid A and Lipid B formulations were shown to be highly stable.

### Example 2: Influenza HIN1 LNP Vaccine Formulations

Influenza pandemics can occur when a novel influenza virus emerges in the human population. Such pandemics remain a major threat to public health, requiring vigilant attention and preparedness with countermeasures to be used in the event of sustained human-to-human spread of the virus. In the experiments described in this Example, hemagglutinin (HA) from a highly pathogenic H1N1 strain A/California/7/2009 (CA09), the cause of the 2009 flu pandemic, was used as a prototype antigen to evaluate the potency of mRNA vaccines prepared with LNP formulations of Lipid A and Lipid B.

The HA mRNA was prepared as described above. Citrate buffer (1 mM citrate, 150 mM NaCl, pH 4.5) was used in the preparation of the LNP compositions. A citrate buffer containing the mRNA was mixed with the lipids in ethanol solution during the formulation process. The pH and the concentration of the buffer were selected to achieve the high encapsulation rate of mRNA in the LNP formulations. The two solutions (mRNA in citrate buffer and lipids in ethanol solution) were mixed in a “T” mixer using a pump system, resulting in a homogeneous pulseless flow, wherein the lipids and the mRNA were mixed at a constant ratio throughout the process. This was critical to achieve a homogeneous formulation with the desired size and a low PDI, an indicator of a more homogeneous size distribution. This process resulted in high mRNA encapsulation, which is critical for achieving high potency. The resultant solution was then subjected to buffer exchange using TFF/dialysis tubes.

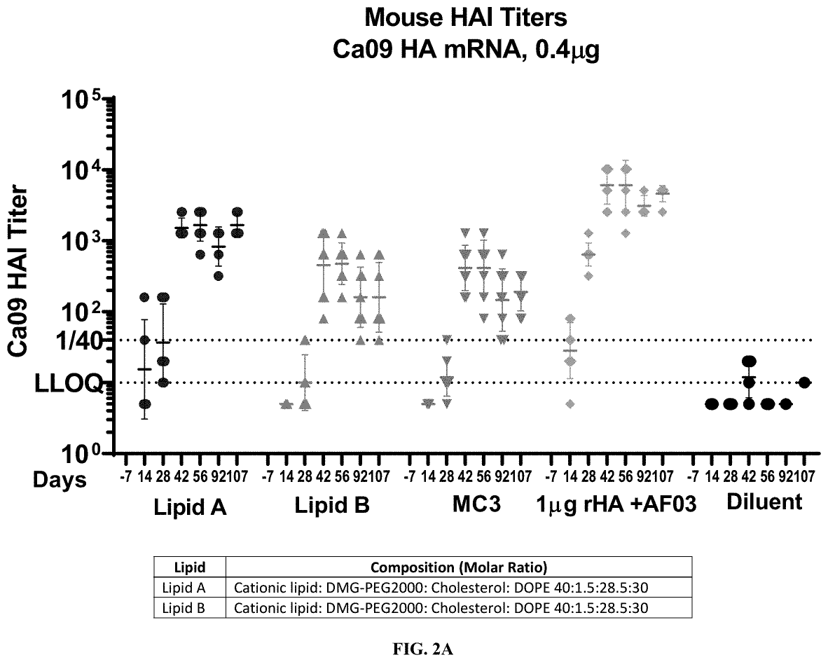

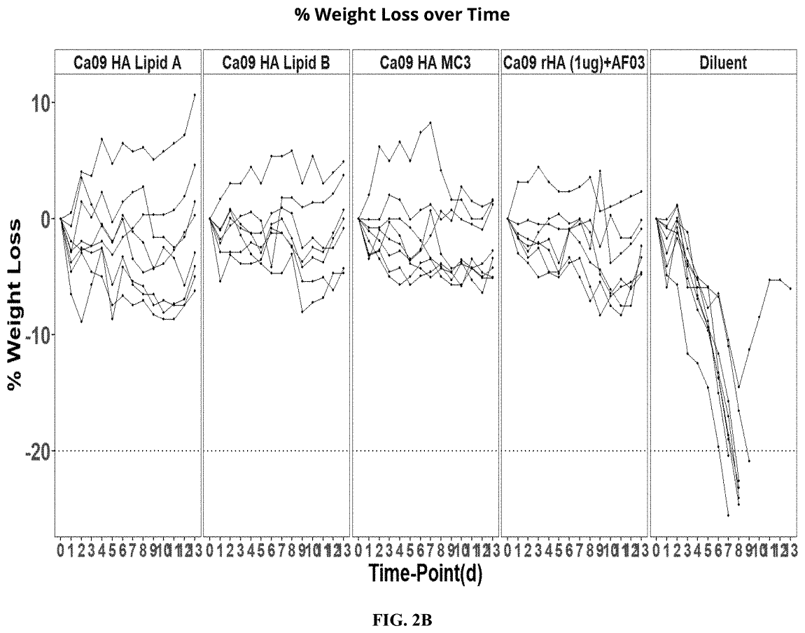

In a mouse study, efficacy of Lipid A and Lipid B CA09 HA formulations were assessed in a head-to-head comparison to MC3 LNP formulation as well as recombinant HA (rHA). CA09 (H1) HA mRNA (0.4 μg) formulated with different cationic lipids was injected intramuscularly into Balb/C mice (n=8) on day 0 (DO) and day 28 (D28). Immunogenicity of the vaccines, as indicated by HA inhibition (HAI) titers, is shown in FIG. 2A. The data show that two immunizations of Lipid A or Lipid B on day 0 (DO) and day 28 (D28) elicited high HAI titers and allowed complete protection of animals from homologous viral challenge (Belgium09 H1N1 virus) (FIG. 2B). During 14 days of post challenge observation, no obvious signs of morbidity (weight loss) were observed within the Lipid A and Lipid B treated groups, while a small number of animals within the recombinant protein control group demonstrated morbidity (FIG. 2B).

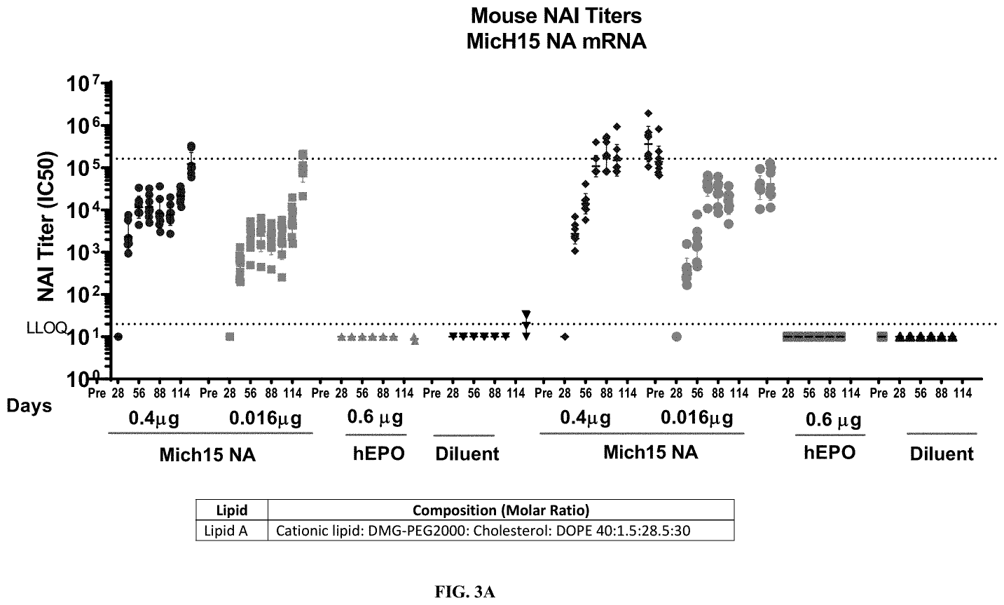

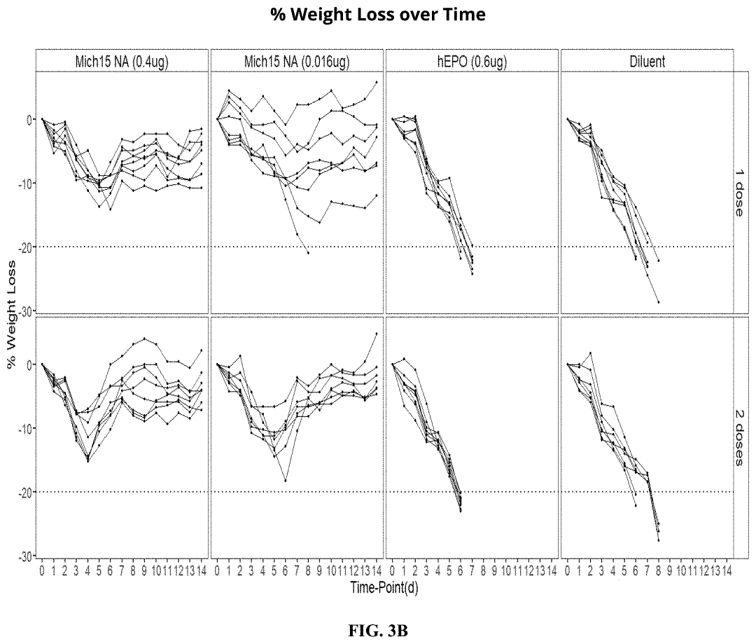

Similarly, mRNA encoding neuraminidase (NA) from the Mich15 influenza strain (Mich15 N1) was formulated with Lipid A and evaluated for its potency. Two doses (0.4 or 0.016 μg) of NA mRNA formulated with Lipid A were injected intramuscularly into Balb/c mice (n=8). The control groups (n=8) were injected with 0.6 μg of hEPO mRNA or with diluent. Half of the mice received only one injection (1 dose) on study day 0, while the other half received two injections (2 doses) given at study day 0 and day 28. The data show that this N1 Lipid A formulation elicited robust immune response, as indicated by NA inhibition (NAI) titers (FIG. 3A). The data further show that the mice treated with either one dose or two doses of the vaccine were protected from lethal viral challenge by Belgium09 H1N1 (FIG. 3B). The level of protection correlated with the NAI titers of vaccine treatment groups versus the negative control groups (hEPO and diluent).

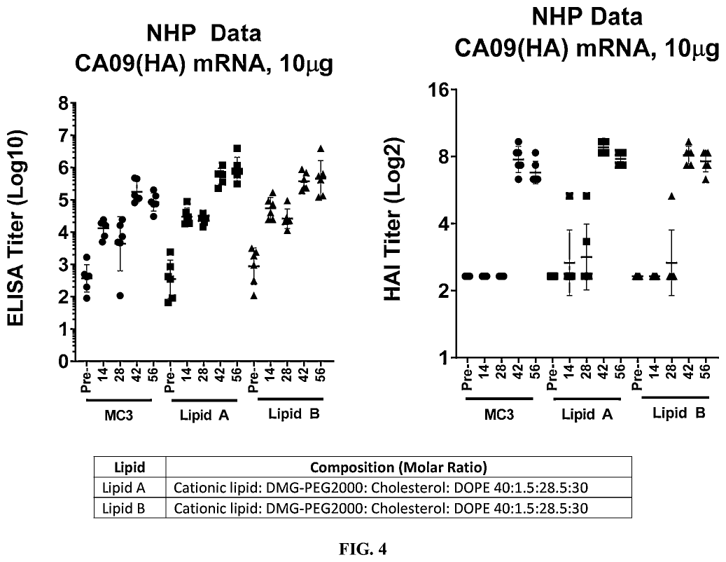

The CA09 H1 mRNA formulated with the present LNPs was also tested in an NHP model. The mRNA (10 μg) was formulated with Lipid A and Lipid B, and injected intramuscularly into cynomolgus macaque monkeys (n=6) on study days 0 and 28. Detectable HAI priming by day 14 and a significant boost in HAI titer by day 28 for all LNPs were observed (FIG. 4, right panel). ELISA data also demonstrated significant priming over baseline by day 14 for all doses tested with a robust boost detected two weeks after the boost (FIG. 4, left panel). The results show that the present H1 mRNA formulations resulted in robust immune responses as indicated by HAI and endpoint ELISA titers.

### Example 3: Influenza H3N2 LNP Vaccine Formulation

This Example describes experiments in which mRNA-LNP vaccine formulations for influenza strain Sing16 (H3N2) were evaluated for potency. One of the mRNAs used in these experiments is MRT1400. MRT1400 is a biosynthetic codon-optimized HA-H3 (influenza virus hemagglutinin, H3 subtype) messenger RNA (CO-HA-H3 mRNA) manufactured by in vitro transcription.

The protein sequence for influenza virus hemagglutinin, H3 sub type, is shown below:

(SEQ ID NO: 1)
MKTIIALSYI LCLVFAQKIP GNDNSTATLC LGHHAVPNGT
IVKTITNDRI EVTNATELVQ NSSIGEIGDS PHQILDGENC
TLIDALLGDP QCDGFQNKKW DLFVERSKAY SNCYPYDVPD
YASLRSLVAS SGTLEFKNES FNWTGVTQNG TSSACIRGSS
SSFFSRLNWL THLNYTYPAL NVTMPNKEQF DKLYIWGVHH
PGTDKDQIFL YAQSSGRITV STKRSQQAVI PNIGSRPRIR
DIPSRISIYW TIVKPGDILL INSTGNLIAP RGYFKIRSGK
SSIMRSDAPI GKCKSECITP NGSIPNDKPF QNVNRITYGA
CPRYVKHSTL KLATGMRNVP EKQTRGIFGA IAGFIENGWE
GMVDGWYGFR HQNSEGRGQA ADLKSTQAAI DQINGKLNRL
IGKTNEKFHQ IEKEFSEVEG RVQDLEKYVE DTKIDLWSYN
AELLVALENQ HTIDLTDSEM NKLFEKTKKQ LRENAEDMGN
GCFKIYHKCD NACIESIRNE TYDHNVYRDE ALNNRFQIKG
VELKSGYKDW ILWISFAISC FLLCVALLGF IMWACQKGNI
RCNICI*

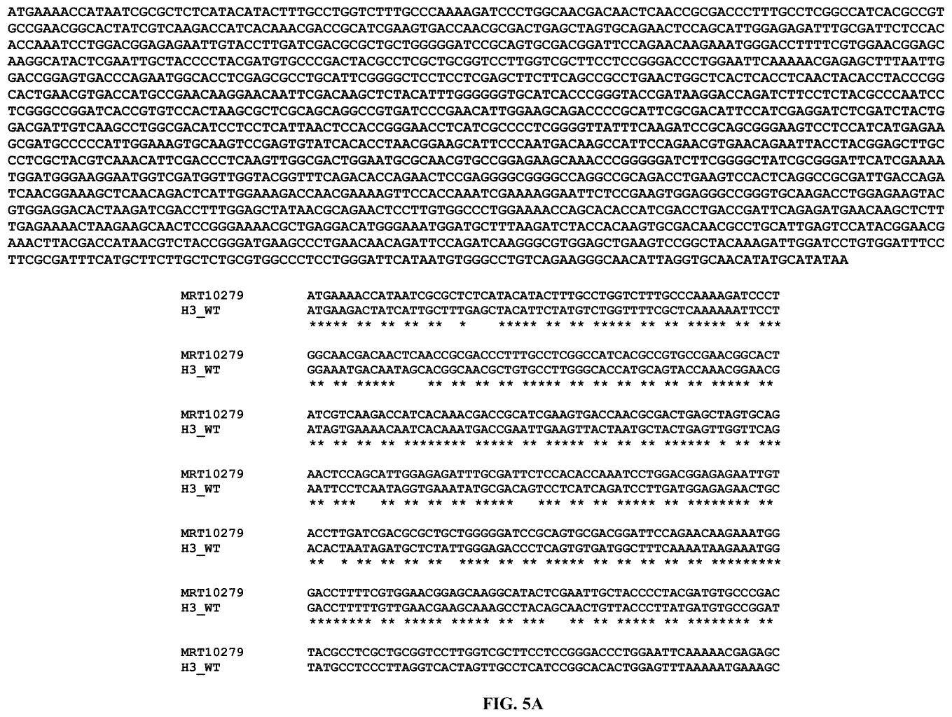

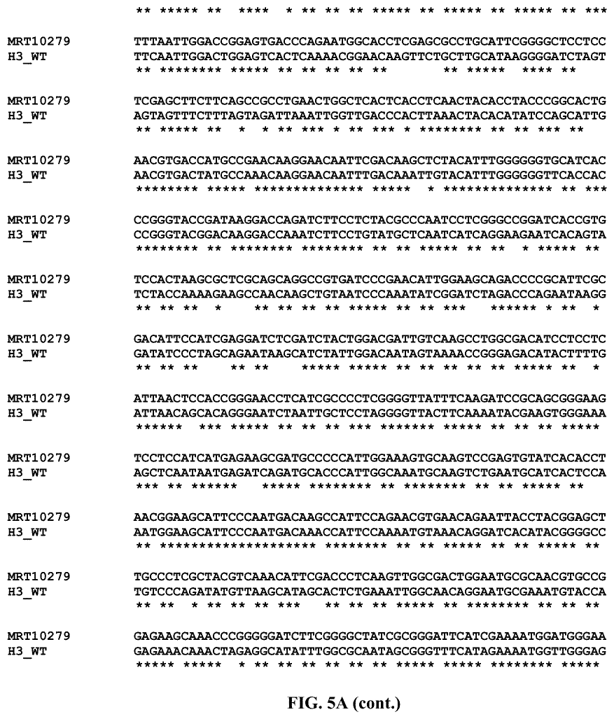

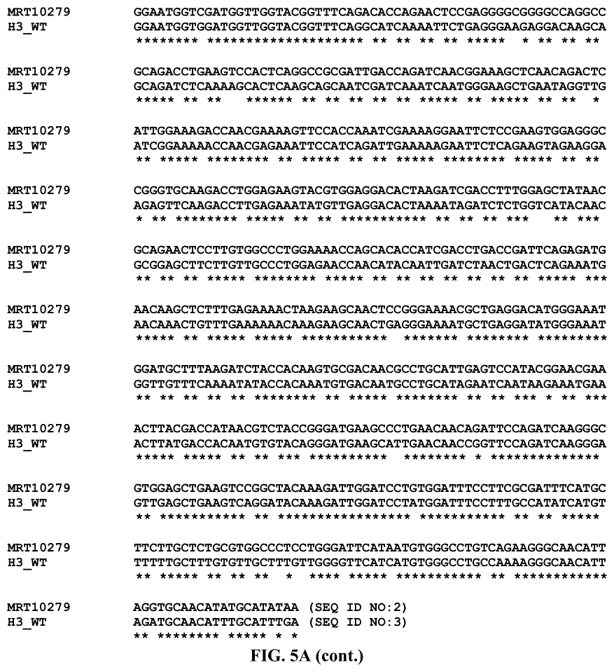

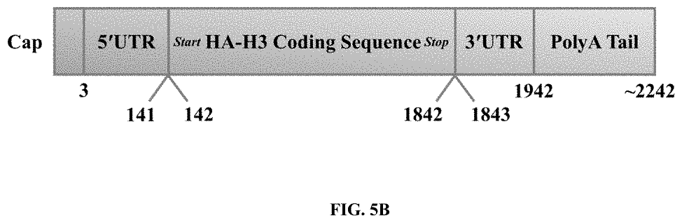

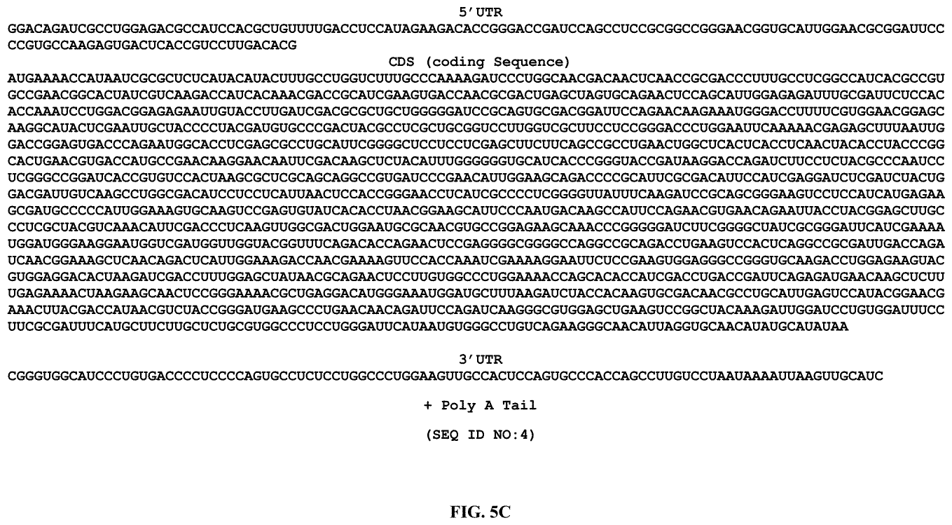

The coding sequence for this protein was codon-optimized. The codon-optimized sequence encoding the protein is shown in FIG. 5A (SEQ ID NO:2), where the wildtype sequence is shown as SEQ ID NO: 3. The mRNA structure and sequence are shown in FIGS. 5B and 5C, respectively. As shown in the figures, the HA-H3 mRNA coding sequence is flanked by 5′ and 3′ untranslated regions (UTRs) of 140 and 100 nucleotides, respectively. The biosynthetic HA-H3 mRNA also contains a 5′ cap structure consisting of a 7-methyl guanosine (m⁷G) residue linked via an inverted 5′-5′ triphosphate bridge to the first nucleoside of the 5′ UTR, which is itself modified by 2′-O-ribose methylation. The 5′ cap is essential for initiation of translation by the ribosome. The entire linear structure is terminated at the 3′ end by a tract of approximately 100 to 500 adenosine nucleosides (polyA). The polyA region confers stability to the mRNA and is also thought to enhance translation. All of these structural elements are naturally occurring components used to promote the efficient translation of the HA-H3 mRNA.

A DNA plasmid was constructed for producing the codon-optimized mRNA sequence by in vitro transcription. In vitro transcription (IVT) reaction was carried out using RNA polymerase. The reaction mixes were precipitated. The precipitated RNA samples were loaded onto individual depth filtration cassette, washed with 80% ethanol and re-dissolved with recirculating H₂O. A second aliquot of H₂O was pumped through in a manner similar to the first step. This step was repeated one more time. The pooled eluates were subjected to ultrafiltration/diafiltration using a 50 kD hollow fiber TFF cassette. Each IVT TFF pool was then diluted in preparation for cap and tail reactions. Cap-tail reactions were precipitated and the RNA from the reaction was purified and collected as described above. The filtered mRNA was stored at −20° C. until use.

In these experiments, mRNA encoding Sing16 NA (N2) or Sing16 HA (H3; MRT1400 mRNA) antigens was formulated with Lipid A or Lipid B LNPs and injected intramuscularly into Balb/c mice (n=8) on DO and D28 at 0.4 μg of mRNA per dose. For comparison, 1 μg of recombinant Sing16 H3 or Sing16 N2 protein with an oil-in-water emulsion adjuvant (AF03) was injected by the intramuscular route into Balb/c mice (n=8). Immune responses were measured by NAI and HAI assays.

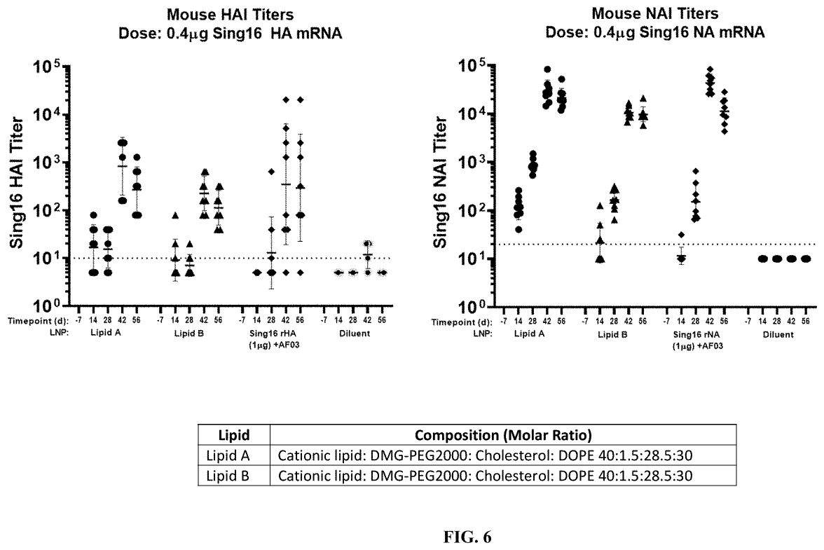

The data show that animals immunized with NA (N2) mRNA demonstrated detectable NAI priming by day 14 and a significant boost in NAI titer by day 28 (FIG. 6, right panel). The data also show that HA Sing16 Lipid A and Lipid B formulations elicited robust HAI responses after boosting on day 28 (FIG. 6, left panel).

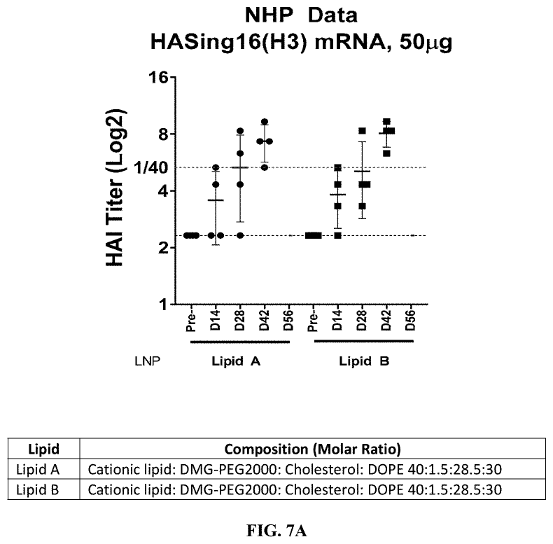

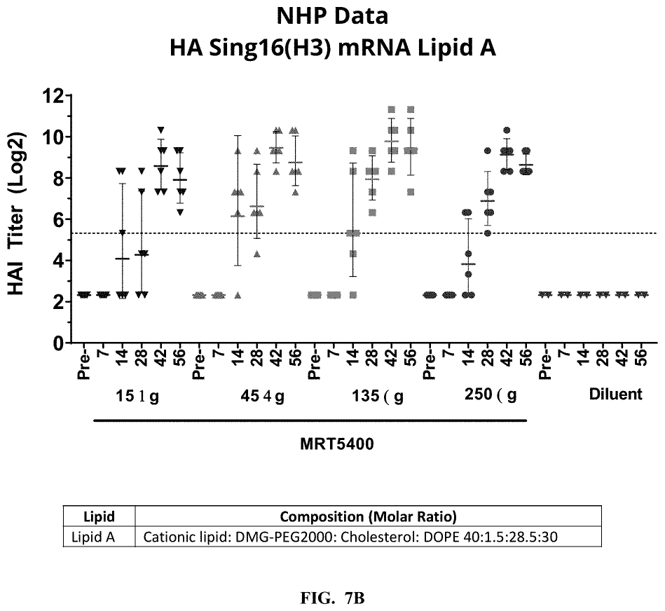

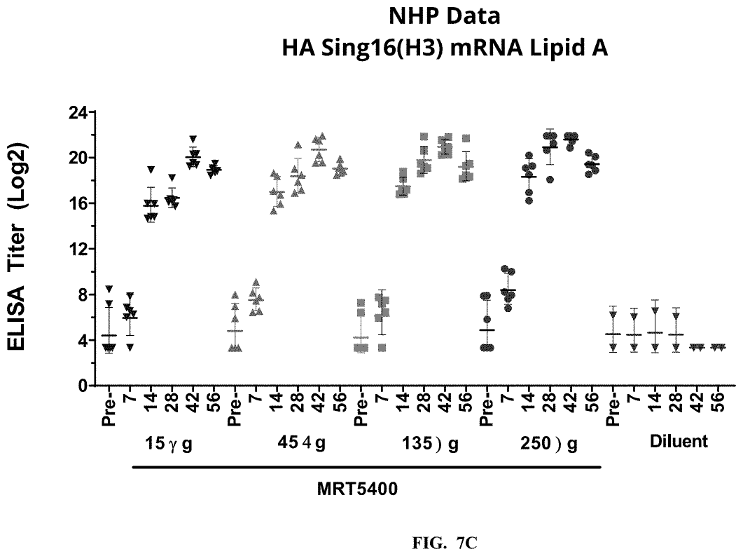

Similarly, the Sing16 HA mRNA Lipid A and Lipid B vaccines were evaluated in non-human primates (NHPs), cynomolgus macaque monkeys (n=6). The HA Sing 16 mRNA (50 μg) formulated with Lipid A or Lipid B was injected by the intramuscular route into the monkeys. The first injection was given at study day 0 and the second injection was given at study day 28. The data show that the vaccines elicited robust immune functional responses boosted on day 28 (FIG. 7A).

In addition, four dose levels of HA Sing16 mRNA formulated in Lipid A (i.e., MRT5400 vaccine)—15, 45, 135 and 250 μg—were evaluated in NHPs. The first immunization was given at study day 0 second immunization at study day 28. All NHPs demonstrated IgG binding and HAI titers for all doses tested with no differences in immune response between the various doses tested at two weeks after the second injection at D42 (FIGS. 7B and 7C).

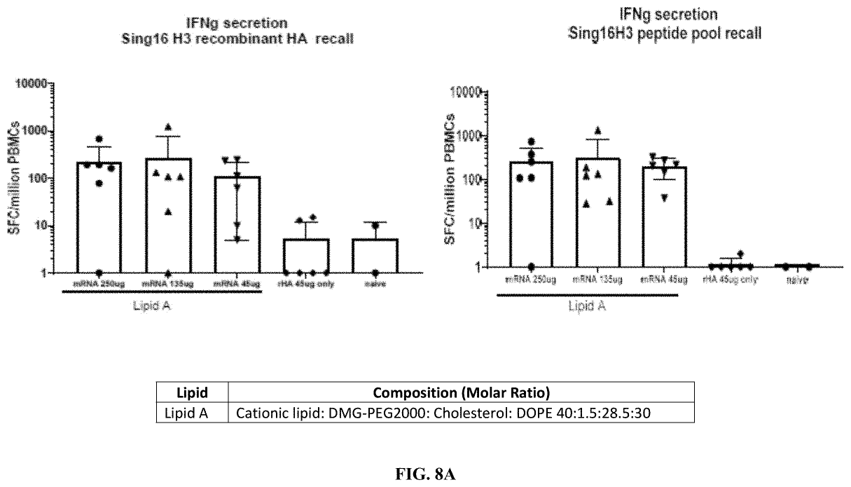

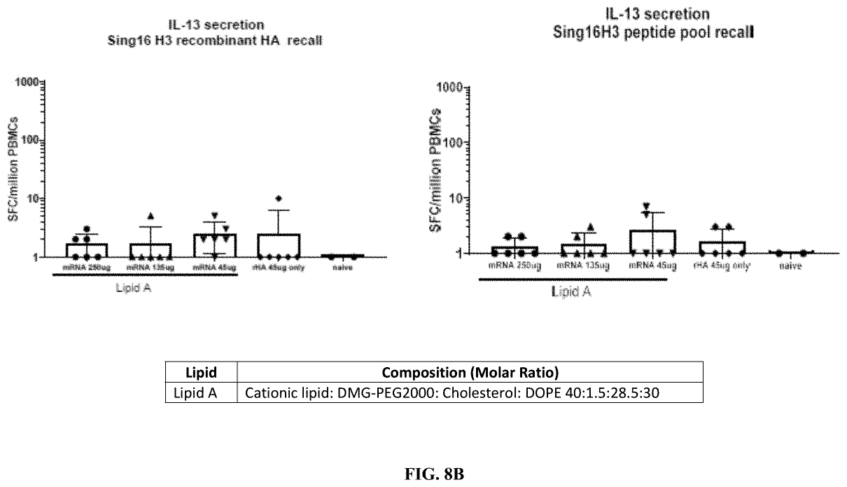

The Sing16 HA mRNA Lipid A vaccine was also evaluated for a T cell response in NHPs after the second vaccination. Peripheral blood mononuclear cells (PBMCs) were collected at day 42 and incubated overnight with either the Sing16 H3 recombinant protein or the peptide pools representing the entire HA open reading frame. Cytokines induced by the re-stimulation were assessed in ELISPOT assays. The frequencies of PBMC secreting IFN-γ, a Th1 cytokine (FIG. 8A), or IL-13, a Th2 cytokine (FIG. 8B) were calculated as spot-forming cells (SFC) per million PBMC. The majority of animals in the three dose level groups tested (250 μg, 135 μg, and 45 μg) demonstrated the presence of high frequency of IFN-γ secreting cells, with over 100 SFCs per million PBMCs (FIG. 8A). A dose-response was not observed, as the animals in the lower and higher dose level groups showed comparable frequencies of IFN-γ secreting cells. In contrast, the presence of IL-13 cytokine secreting cells was not detected in any of the groups tested and at any dose level (FIG. 8B). These data presented clear evidence for a Th1-biased cellular response and a lack of Th2 response to the HA antigen following vaccination in NHPs.

### Example 4: Influenza LNP Vaccine Formulations with Modified mRNA

This Example describes experiments comparing the potency of vaccines containing unmodified (unmodified non-replicating or “UNR”) and modified (modified non-replicating or “MNR”) mRNA. UNR CA09 HA mRNA and MNR CA09 HA mRNA were prepared by in vitro transcription. In MNR, all uridines were replaced by pseudouridines.

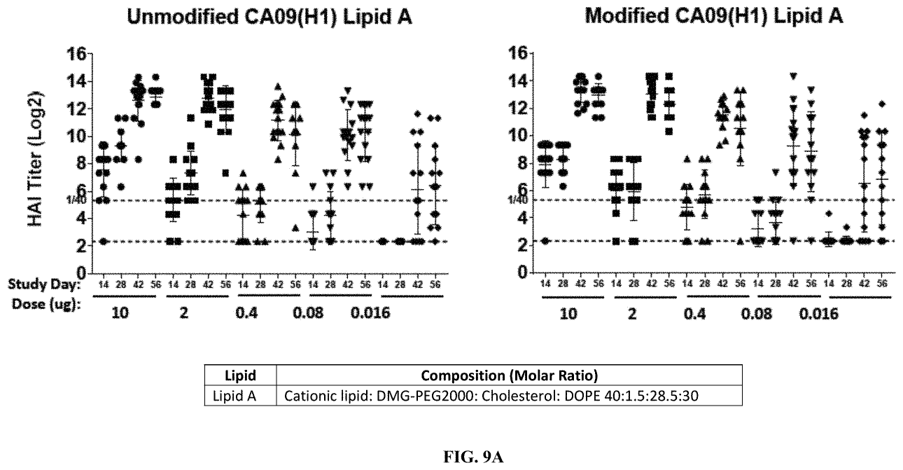

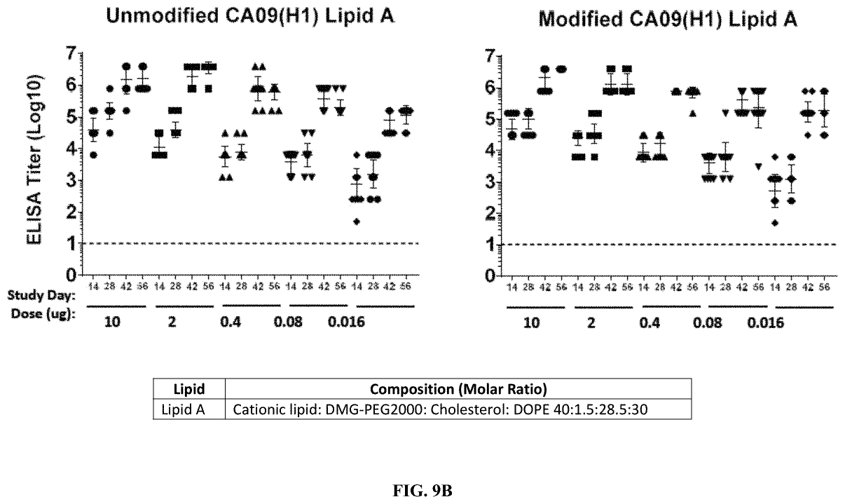

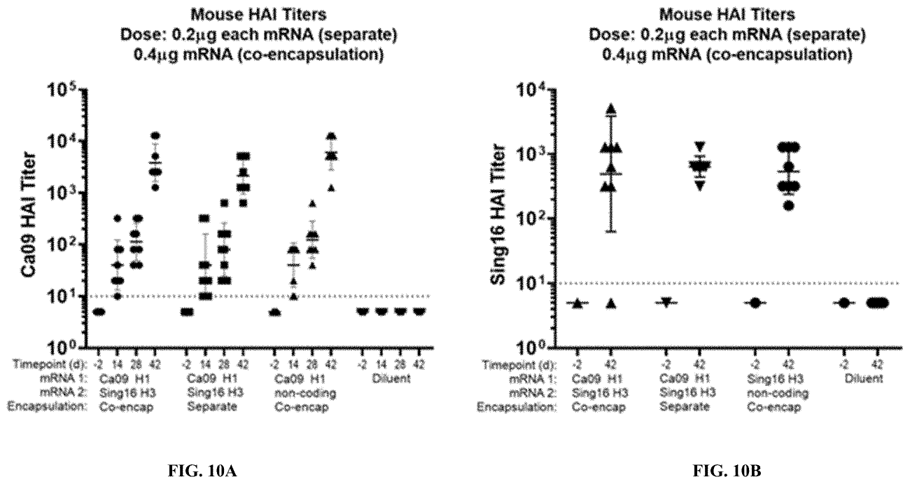

Five different doses (0.016, 0.08, 0.4, 2, and 10 μg) of CA09 HA mRNA (either modified or unmodified) formulated with Lipid A were injected by the intramuscular route into Balb/c mice (n=15). The data show that the LNP formulations increased the stability and delivery efficiency of naked mRNA (UNR), for the potency between UNR and MNR mRNA was comparable as indicated by HAI titers (FIG. 9A). ELISA data for Balb/c mice also demonstrated significant priming over baseline by day 14 for all doses tested (both UNR and MNR mRNAs), with a robust boost detected two weeks after the boost. The data also show that UNR and MNR mRNAs were comparable in eliciting ELISA titers (FIG. 9B).

In conclusion, the present dose titration study demonstrated that unmodified and modified CA09 HA mRNA formulated with Lipid A elicited statistically indistinguishable immune responses in Balb/c mice, as indicated by either HAI or by endpoint ELISA assay. Balb/c mice immunized with the four higher doses of UNR and MNR mRNA demonstrate detectable HAI priming by day 14 and a significant boost in HAI titer by day 42 for all doses. These day-14 priming titers represent both a dose effect and dose sparing potential for generating detectable titers over a 125-fold range. The second injection titers at the same dose range confirms the robustness of the immune response to this mRNA-LNP formulation. Similar results were also observed in non-human primates.

### Example 5: Multi-Valent Influenza Vaccine LNP Formulation

This Example describes a study using a Lipid A-based LNP vaccine containing mRNA encoding CA09 HA (as described in Example 2) and mRNA encoding Sing16 HA (as described in Example 3).

More specifically, CA09 HA mRNA and Sing16 HA mRNA co-encapsulated in Lipid A were evaluated in Balb/c mice (n=8). mRNA-LNP was administered as two mRNAs co-encapsulated or dosed separately as singly encapsulated mRNAs. For both approaches, a total of 0.4 μg LNP formulation was injected into mice by intramuscular injection. The first injection was given at study day 0 and the second injection was given at study day 28. The data show that the vaccines elicited robust immune functional responses. There did not appear to be any difference between the two administration approaches. These data show that co-encapsulation did not cause hindrance or interference between the two mRNAs.

### Example 6: Further Studies on Multi-Valent Influenza Vaccine LNP Formulations

A panel of unmodified mRNAs encoding CA09 HA, Sing16 HA, Sing16 NA, Mich15 NA, A/Perth/16/2009 influenza virus (Perth09 NA), and reporter antigens of firefly luciferase (FF) and hEPO were prepared. LNP formulations for HA and NA mRNA-LNP preparation were then tested for expression in vitro, the immune responses in animals, and for potency in preclinical models. For the studies in this Example, all of the LNP formulations were the Lipid A formulation.

### Materials and Methods

mRNA-LNP Preparations

mRNA transcripts encoding for hEPO, FF, CA09 HA, Sing16 HA, Mich15 NA, and Sing16 NA were synthesized by in vitro transcription employing RNA polymerase with a plasmid DNA template encoding the desired gene using unmodified nucleotides. The resulting purified precursor mRNA was reacted further via enzymatic addition of a 5′ cap structure (Cap 1) and a 3′ poly(A) tail of approximately 200 nucleotides in length as determined by gel electrophoresis and purified. All mRNA preparations were analyzed for purity, integrity, and percentage of Cap 1 before storage at −20° C. Preparation of mRNA/lipid nanoparticle (LNP) formulations was described above. Briefly, an ethanolic solution of a mixture of lipids (ionizable lipid, phosphatidylethanolamine, cholesterol and polyethylene glycol-lipid) at a fixed lipid and mRNA ratio were combined with an aqueous buffered solution of target mRNA at an acidic pH under controlled conditions to yield a suspension of uniform LNPs. Upon ultrafiltration and diafiltration into a suitable diluent system, the resulting nanoparticle suspensions were diluted to final concentration, filtered, and stored frozen at −80° C. until use. The mRNA-LNP formulations were characterized for size by dynamic light scattering, percentage encapsulation and were stored at −80° C. at 1 mg/mL until further use by dilution with suitable buffer. hEPO-LNPs and FF-LNPs were utilized to check level of expression of target protein in vivo.

Visualization of S-Proteins Expressed in HeLa Cells

Immunocytochemistry-immunofluorescence analysis of influenza NA and HA-proteins was performed in HeLa cells transfected with bivalent H3N2 (Sing16 HA and Perth09 NA) mRNAs LNPs) using method described previously (Kalnin et al., npj Vaccines (2021) 6:61). Cells were fixed in 4% paraformaldehyde and subjected antibody staining for HA (GeneTex GTX40258), NA, and ER marker Calnexin (Abcam ab22595) was performed. Images were captured on confocal microscope followed by image analysis for quantification of HA and NA colocalization to the ER, mean signal intensity, and percent of cell area.

Flow Cytometry

Human skeletal muscle cells (HskMCs, Lonza) were cultured in M199 (Life Technologies) supplemented with GlutaMAX (Life Technologies), streptomycin, penicillin (Gibco), and 20% heat inactivated FBS (VWR) at 37° C. with 5% CO₂. The cells were harvested by trypsinization, washed with PBS, and electroporated using human primary muscle cell transfection kit on Nucleofector 2b (Lonza) with 12 mg of mRNA per 10⁶ cells following manufacturer's electroporation program D-033. Post 24 hour harvested cells were fixed, permeabilized with CYTOFIX™/Perm (BD) and stained with CA09 HA (Immune Tech), Sing16 HA (30-2F11-F7-A5, GeneTex), Mich15 NA (6G6, Immune Tech) and Sing16 NA (40017-RP01, Sino Biologicals) specific Ab followed by PE conjugated goat anti-mouse IgG secondary Ab (Southern Biotech) or AF647 conjugated goat anti-rabbit IgG (Life Technologies). Then the antibody-labeled cells were acquired by Fortessa (BD) and the expression of each protein was analyzed by FLOWJO™ (TreeStar).

Cryogenic Transmission Electron Microscopy

A PELCO EASIGLOW™ device was used to plasma-clean the grids prior to LNP sample application, and a Vitrobot Mark IV System (ThermoFisher) with the chamber held at 100% humidity and 18° C. was used for plunge freezing. A 3.0 μl droplet of LNP sample was dispensed onto 300 mesh R2/1 QUANTIFOIL® grids with carbon film and gold bars. Grids were blotted for 4 seconds, held in place for 10 seconds, and then immediately plunge frozen in liquid ethane for storage and transfer to a Krios microscope. Exposures were collected using a Titan Krios transmission electron microscope (ThermoFisher) equipped with a BioQuantum energy filter and K3 direct electron detector (Gatan) operating in counting mode. Calibrated physical pixel size at the detector was 1.38 Å, corresponding to 64,000× magnification. A total of 3,141 69-frame movie exposures were collected at a dose per frame of 1.045 e/Å2 with defocus between −0.5 to −1.7 μm. For each movie exposure, patch-based motion correction, binning of super-resolution pixels, and frame dose-weighting was performed using RELION-3.1.34. From corrected images, over 700 candidate particle coordinates were extracted. Subsequent data analysis was done with MATLAB R2019a with image processing toolbox.

Immunization of Mice and NHPs for Expression Studies

Groups of four cynomolgus macaques (NHPs) (male and female) and four to eight male BALB/c mice were administered intramuscularly either dose of 10 μg (NHP) or 1, 0.5, 0.1, and 0.05 μg (mice) with hEPO-LNP prepared in the same ratio as the one intended to be used for HA/NA mRNA-LNP formulations. Blood samples were taken pre-administration, and at 6 h, 24 h, 48 h, 72 h, and 96 h post administration to monitor for serum hEPO expression via an ELISA using R and D Systems, QUANTIKINE® IVD® ELISA, Human Erythropoietin Immunoassay kit as per manufacturers protocol, and reported as final values of mIU/ml and ng/ml. Briefly, microplate wells, precoated with a mouse monoclonal antibody specific for EPO were incubated with specimen or standard. After removing excess specimen or standard, wells were incubated with a rabbit anti-EPO polyclonal antibody conjugated to horseradish peroxidase. During the second incubation, the antibody-enzyme conjugate bound to the immobilized EPO. Excess conjugate was removed by washing. A chromogen was added to the wells and was oxidized by the enzyme reaction to form a blue colored complex. The reaction was stopped by the addition of acid, which turned the blue to yellow. The amount of color generated was directly proportional to the amount of conjugate bound to the EPO antibody complex, which, in turn, was directly proportional to the amount of EPO in the specimen or standard. The absorbance of this complex was measured, and a standard curve was generated by plotting absorbance versus the concentration of the EPO standards. The EPO concentration of the unknown specimen was determined by comparing the optical density of the specimen to the standard curve. The standards used in this assay were recombinant hEPO calibrated against the Second International Reference Preparation (67/343), a urine-derived form of human erythropoietin.

Immunization of Mice and NHPs for Immunogenicity Studies

Groups of Balb/c mice (Mus musculus) as per treatment group were immunized under isoflurane anesthesia with a dose of 0.05 mL of designated vaccine preparation or diluent via the IM route in the quadriceps, on day 0 in one hind leg and day 28 in the contralateral leg. Mice that lost more than 20% of their initial body weight and displayed severe clinical signs were euthanized after the veterinarian's assessment of the animal's health prior to the study termination.

Naïve male and female Mauritius origin Cynomolgus macaques (Macaca fascicularis) were selected for the study. Animals weighed >2 kg and were >2 years of age at the start of the study. Animals selected for the study underwent comprehensive physical examinations prior to assignment to the study. The pre-assignment assessment of health status included a hands-on veterinarian examination and blood sample collections for CBC analysis as applicable per NIRC SOPs. Animals were generally housed in pairs and acclimated for at least 3 days prior to the start of the study. Groups consisted of up to 6 animals per treatment group. All animals were immunized under ketamine HCl (10 mg/kg, IM) or telazol (4-8 mg/kg, IM) sedation with a dose of 0.5 ml of their respected vaccine preparation or diluent via the IM route in one forelimb of each animal, targeting the deltoid, on Study Day 0. Twenty-eight days after the first immunization took place, a second immunization was given to the animals in the contralateral limb.

Immunization of Mice and NHPs for Challenge Studies

Mice were inoculated with the challenge strain approximately 9-12 weeks after the last immunization. Vials of stock virus were thawed and diluted to the appropriate concentration in ice-cold sterile PBS. All mice were challenged with a total volume of 50 μl containing 105.54 TCID₅₀ of Belgium09 virus in PBS which equated to 4LD₅₀. Virus challenge was performed inside the biosafety cabinet in an enhanced ABSL2 laboratory. Mice were first anesthetized with an IP injection of a Ketamine/Xylazine solution (50 mg/kg Ketamine and 5 mg/kg Xylazine), and then challenged IN (dropwise into both nostrils; 25 μl per nostril) with a total volume of 50 μl of influenza virus using a micropipette. Following the challenge procedure, mice were placed in dorsal recumbency and observed until recovery from anesthesia. Daily body weights were taken following H1N1 challenge. Any individual animal with a single observation >20% body weight loss was euthanized. The weight measurements were either recorded daily post challenge until euthanasia in the online database, PRISTIMA® (Version 7.5.0 Build 8), or written on study specific working sheets.

Blood Collection

For mice, blood was collected via submandibular or orbital sinus bleeds (in-life bleed, pre-study and on study days 14, 28, and 42 approximately 200 μl) and cardiac puncture (terminal bleed, day 56) from all animals under sedation. Mice were bled on pre-study to obtain a base-line pre-immune serum sample and for pre-screening purposes. Processing of the serum, blood samples were collected into SST tubes and allowed to clot for 30 minutes to 1 hour at room temperature. The samples were then centrifuged 1000-1300 g for 5-10 minutes with brakes off. Serum was collected using a P200 pipettor, divided into two 0.5 ml cryovials, and stored at −20° C. All bleeds were documented on specimen collection and processing logs, indicating the time of sample collection and the technician responsible for performing the procedure. A portion of the serum samples were evaluated in the HAI or ELLA and ELISA assays for antibody titers.

NHPs were bled for serum isolation while under anesthesia administered intramuscularly using 10 mg/kg ketamine/1 mg/kg acepromazine (days −4, 2, 7, 14, 28, 30, 35, 42, 56, 90, and 180). The volume of blood withdrawn did not exceed established guidelines with respect to percentage of body weight and animal's physical condition. Blood was withdrawn from anesthetized NHPs using femoral venipuncture using a Vacutainer 21 ga×1″ blood collection needle or Abbott Butterfly 23 ga×¾″ tubing attached to BD Vacutainer® SST™ gel tubes. Serum was isolated by spinning the tubes at room temperature at a speed of 1200×g for 10 minutes. Serum was then aliquoted into labeled cryovials (1 ml/vial) and stored at ≤−20° C. A portion of the serum samples were evaluated in the HAI or ELLA and ELISA assays for antibody titers. For PBMCs, NHPs were pre-bled before vaccination and again approximately 42-63 days after the first injection. For this purpose, blood was collected into BD Vacutainer® tubes containing heparin anticoagulant. Briefly, anticoagulated blood samples were diluted in PBS and subjected to gradient density centrifugation for 30 minutes at 400×g using HISTOPAQUE® separation solution (Sigma). The opaque interface containing mononuclear cells was then collected, washed three times in PBS using a low speed (250×g) centrifugation for the last centrifugation to reduce the number of platelets. The live vs. dead PBMC were enumerated using a Nexcelom Cellometer K2. The PBMC were cryopreserved in FBS with 10% DMSO using MR. FROSTY® freezing boxes. The boxes were placed immediately into a −80° C. freezer for 24 hours and then transferred for storage in a liquid nitrogen tank.

ELISA

The antibody ELISAs were performed using recombinantly produced Sing16 NA protein, Sing16 HA protein, or CA09 HA protein. The proteins were captured on 96 well high binding polystyrene plates at a concentration of 2 μg/ml in carbonate-bicarbonate buffer. The plates were covered and incubated overnight (16±4 hours) at 2-8° C. After overnight incubation, the antigen coated plates were washed 5 times with a washing buffer (PBS, 0.5% Tween20) and blocked with a blocking solution (10% BSA in PBS) for 60±30 minutes at room temperature. Test samples, naïve control, and the reference sample were diluted in a sample diluent (PBS 10% BSA 0.5% Tween 20) and added to wells in duplicates followed by incubation at room temperature for 90 minutes. Plates were washed 5 times with the washing buffer, and goat anti-mouse HRP for mouse sera or goat anti-monkey HRP for NHP sera was added at a dilution of 1:10,000. The plates were then incubated 30 minutes at room temperature and the excess HRP-IgG was washed with the washing buffer. Sure-Blue TMB substrate was added to each plate and the reaction was stopped after about 10 minutes with TMB stop solution. The plates were then read at 450 nm with a Thermo Labsystems MULTISKAN™ spectrophotometer. The anti-antigen (HA or NA) specific antibody titers were expressed as a reciprocal of the highest serum dilution with an absorbance value >0.3.

HAI Assay

HAI assays were performed using the Sing16 H3N2 and the CA09 H1N1 virus stocks (BIOQUAL, Inc.). Sera were treated with receptor-destroying enzyme (RDE) by diluting one-part serum with three parts enzyme and incubated overnight in a 37° C. water bath. Enzyme was inactivated by a 30-minute incubation period at 56° C. followed by addition of six parts PBS for a final dilution of 1/10. HAI assays were performed in V-bottom 96-well plates using four hemagglutinating units (HAU) of virus and 0.5% turkey RBC. The reference serum for each strain was included as a positive control on every assay plate. Each plate also included a back-titration to confirm the antigen dose (4 HAU/25 μl) as well as a negative control sample (PBS or naïve control serum). The HAI titer was determined as the highest dilution of serum resulting in complete inhibition of hemagglutination. Results were only valid for plates with the appropriate back-titration result (verifying 4 HAU/25 μl added) and a reference serum titer within 2-fold of the expected titer.

NAI Assay

The method for the enzyme-linked lectin assay (ELLA) assay was used to determine neuraminidase-inhibiting (NAI) antibody titers. The source of antigen (virus NA) was titrated, and a standard amount was selected for incubation with serial dilutions of serum. Titration of sera was performed with serial dilutions of sera (heat inactivated at 56° C. for 1 hour) and a standard amount of virus was added to duplicate wells of a fetuin-coated plate. This mixture was then incubated overnight (16-18 hours); the next day, HRP-conjugated peanut agglutinin PNA (diluted to 2.5 μg/ml) was added to the washed plate and incubated for 2 hours at room temperature. Substrate (ODP in sodium citrate) was added and incubated for 10 minutes to develop the color. And then stop buffer (1N sulfuric acid) was added to stop the reaction. Plates were scanned for absorbance at OD 490 nm. The reduction or absence of color relative to a viral control indicated inhibition of NA activity due to the presence of NA-specific antibodies. NAI titers (IC₅₀ values) were calculated from the OD readings and the results were graphed in GraphPad Prism. If ELLA titration curves did not allow a good fit to determine a reliable IC50 value, the samples were retested using a different dilution scheme to reach the 50% endpoint.

T Cell ELISPOT Assay

Complete medium (DMEM1640+10% heat-inactivated FCS) was prewarmed in a 37° C. water bath. PBMCs were quickly thawed in a 37° C. water bath and transferred dropwise to conical tubes with the prewarmed medium. The tubes were centrifuged at 1,500 rpm for 5 inns and the cells were resuspended and counted using a Guava cell counter. Monkey IFN-γ ELISPOT kit (Mabtech 3421M-4APW) and IL-13 ELISPOT kit (Mabtech 3470M-4APW) were used. Precoated plates provided by the kits were washed four times with sterile PBS and blocked with 200 μl of complete medium in 37° C. incubator for at least 30 minutes. Sing16 H3 peptides pool (Genscript Custom Order) (at 1 μg/ml of each peptide) were used as recall antigens in the assay. Two μg/ml of ConA (Sigma CAT #C5275) was used as a positive control. Fifty μl of recall antigens and 300,000 of PBMCs in 50 μl were added to each well for stimulation. The plates were placed in a 37° C., 5% CO₂ humidified incubator for 48 hours.

After the incubation, cells were removed, plates were washed 5 times with PBS, and 100 μl of 1 μg/ml biotinylated anti-IFN-γ or anti-IL-13 detection antibodies were added to each well in the plates. After a 2 hour incubation, the plates were washed 5 times with PBS and incubated with 100 μl of a 1:1000 dilution of streptavidin in each well for one hour at room temperature. Plates were developed with 100 μl of BCIP/NBT substrate solution until the spots emerged. Plates were rinsed by tap water, air-dried and scanned and counted using CTL IMMUNOSPOT® Reader (Cellular Technology Ltd.). The data was reported as spots forming cells (SFC) per million PBMCs.

Memory B Cell (MBC) ELISPOT Assay

Human IgG Single-Color memory B cell ELISPOT kit (CAT #NC1911372, CTL) was used per manufacturer's instruction to measure Sing16 H3-specific and total IgG⁺ antibody-secreting cells (ASCs). Differentiation of MBCs into ASCs was performed in PBMC using a stimulation cocktail provided by the kit. Briefly, frozen PBMCs were quickly thawed in a 37° C. water bath, mixed with DNase I (CAT #90083, Fisher Scientific) and transferred into the tube containing pre-warmed complete culture medium (CM) (RPMI 1640, (CAT #22400-089, Gibco) containing 10% FCS (CAT #SH30073.03, HYCLONE™), and 1% penicillin/streptomycin (CAT #P4333, Sigma) and centrifuged at 1,500 rpm for 5 minutes. Cell pellet was re-suspended in 5 ml of complete medium at 2×10⁶ cells per ml and transferred to a T25 flask for 1 hour in 5% CO₂ incubator at 37° C. The volume of cell suspension was then adjusted to 6 ml and B-Poly-S was added at 1:1000 dilution. Cells were left in the CO₂ incubator for stimulation for 4 days. PVDF microplates supplied by the kit were pre-wetted with 70% ethanol, rinsed and coated overnight with 80 μl/well of either anti-human IgG capture Ab provided by the kit or Sing16/H3 recombinant protein at 4 μg/ml.

Cells were harvested after 4 days of stimulation, washed, and counted and adjusted to the designated concentration in the CM. Coated microplates were washed with PBS, blocked for 1 hour with the CM and emptied out. Cell suspension at 100 μl/well was added to the plates and incubated in CO₂ incubator at 37 C for 18 hrs. After washing, 80 μl/well of 1:400 diluted anti-human IgG biotin detection antibody was added to the plate and incubated at room temperature for 2 hours. Following washing, Streptavidin-AP at 1:1000 dilution was added to the plate at 80 μl/well for 1 hour. Freshly prepared Substrate solution was added and incubated at RT for 18 min. Plates were rinsed by tap water, air-dried and scanned and counted using CTL IMMUNOSPOT® Reader (Cellular Technology Ltd). For each individual animal, the number of IgG⁺ and number of Sing16/H3-specific ASCs was calculated per million of PBMCs. The frequency of antigen-specific ASCs was calculated as % of antigen-specific ASCs to the total IgG⁺ ASCs. To assess assay background the negative control wells on every plate were coated with PBS (no background was detected).

Statistical Analysis

For estimating the Tₘₐₓ of Radiance, a non-parametric method was used to estimate the Tₘₐₓ of individual subject based on observed data. For estimating the half-life of Radiance, assuming exponential decay model for radiance after reaching the maximum value, a linear model was fitted to log transformed data per subject during the time course from the maximum radiance to decay to baseline (we estimate the baseline using the average of radiance in saline group). The half-life was estimated as the time point when the log radiance had reached the middle point between maximum and baseline values. For analysis of different readouts with results summarized as geometric mean, SE model based geometric means and SEs were estimated from a mixed effect model for repeated measures where the response was the log transformed readouts, vaccination was fixed effect and time was repeated measure; log-based means and SE estimates from the model were then back transformed to get geometric means and SEs. For weight change, over descriptive statistical analysis was used. Medians and ranges of each group of the maximum % body weight loss from baseline (Day 0) over time were reported to evaluate the worse scenarios; medians and ranges of each group of the % body weight change from baseline at the last observation were reported to evaluate the body weight recovery.

Antigen Sequences

The sequence of the Perth09 N2 antigen used here is:

(SEQ ID NO: 4)
MNPNQKIITIGSVSLTISTICFFMQIAILITTVTLHEKQYEFNSPPNNQV
MLCEPTIIERNITEIVYLTNTTIEKEICPKLAEYRNWSKPQCDITGFAPF
SKDNSIRLSAGGDIWVTREPYVSCDPDKCYQFALGQGTTLNNVHSNNTVR
DRTPYRTLLMNELGVPFHLGTKQVCIAWSSSSCHDGKAWLHVCITGDDKN
ATASFIYNGRLVDSVVSWSKEILRTQESECVCINGTCTVVMTDGSASGKA
DTKILFIEEGKIVHTSTLSGSAQHVEECSCYPRYPGVRCVCRDNWKGSNR
PIVDINIKDHSIVSSYVCSGLVGDTPRKNDSSSSSHCLDPNNEEGGHGVK
GWAFDDGNDVWMGRTISEKSRLGYETFKVIEGWSNPKSKLQINRQVIVDR
GNRSGYSGIFSVEGKSCINRCFYVELIRGRKEETEVLWTSNSIVVFCGTS
GTYGTGSWPDGADINLMPI*

The sequence of the Mich15 N1 antigen used here is:

(SEQ ID NO: 5)
MNPNQKIITIGSICMTIGMANLILQIGNIISIWVSHSIQIGNQSQIETCN
QSVITYENNTWVNQTYVNISNTNFAAGQSVVSVKLAGNSSLCPVSGWAIY
SKDNSVRIGSKGDVFVIREPFISCSPLECRTFFLTQGALLNDKHSNGTIK
DRSPYRTLMSCPIGEVPSPYNSRFESVAWSASACHDGINWLTIGISGPDS
GAVAVLKYNGIITDTIKSWRNNILRTQESECACVNGSCFTIMTDGPSDGQ
ASYKIFRIEKGKIIKSVEMKAPNYHYEECSCYPDSSEITCVCRDNWHGSN
RPWVSFNQNLEYQMGYICSGVFGDNPRPNDKTGSCGPVSSNGANGVKGFS
FKYGNGVWIGRTKSISSRKGFEMIWDPNGWTGTDNKFSIKQDIVGINEWS
GYSGSFVQHPELTGLDCIRPCFWVELIRGRPEENTIWTSGSSISFCGVNS
DTVGWSWPDGAELPFTIDK*

The sequence of the Sing16 H3 antigen used here is:

(SEQ ID NO: 6)
MKTIIALSYILCLVFAQKIPGNDNSTATLCLGHHAVPNGTIVKTITNDRI
EVTNATELVQNSSIGEICDSPHQILDGENCTLIDALLGDPQCDGFQNKKW
DLFVERSKAYSNCYPYDVPDYASLRSLVASSGTLEFKNESFNWTGVTQNG
TSSACIRGSSSSFFSRLNWLTHLNYTYPALNVTMPNKEQFDKLYIWGVHH
PGTDKDQIFLYAQSSGRITVSTKRSQQAVIPNIGSRPRIRDIPSRISIYW
TIVKPGDILLINSTGNLIAPRGYFKIRSGKSSIMRSDAPIGKCKSECITP
NGSIPNDKPFQNVNRITYGACPRYVKHSTLKLATGMRNVPEKQTRGIFGA
IAGFIENGWEGMVDGWYGFRHQNSEGRGQAADLKSTQAAIDQINGKLNRL
IGKTNEKFHQIEKEFSEVEGRVQDLEKYVEDTKIDLWSYNAELLVALENQ
HTIDLTDSEMNKLFEKTKKQLRENAEDMGNGCFKIYHKCDNACIESIRNE
TYDHNVYRDEALNNRFQIKGVELKSGYKDWILWISFAISCFLLCVALLGF
IMWACQKGNIRCNICI*

The sequence of the Sing16 N2 antigen used here is:

(SEQ ID NO: 7)
MNPNQKIITIGSVSLTISTICFFMQIAILITTVTLHEKQYEFNSPPNNQV
MLCEPTIIERNITEIVYLTNTTIEKEICPKPAEYRNWSKPQCGITGFAPF
SKDNSIRLSAGGDIWVTREPYVSCDPDKCYQFALGQGTTLNNVHSNNTVR
DRTPYRTLLMNELGVPFHLGTKQVCIAWSSSSCHDGKAWLHVCITGDDKN
ATASFIYNGRLIDSVVSWSKDILRTQESECVCINGTCTVVMTDGNATGKA
DTKILFIEEGKIVHTSKLSGSAQHVEECSCYPRYPGVRCVCRDNWKGSNR
PIVDINIKDHSIVSSYVCSGLVGDTPRKNDSSSSSHCLNPNNEEGGHGVK
GWAFDDGNDVWMGRTINETSRLGYETFKVVEGWSNPKSKLQINRQVIVDR
GDRSGYSGIFSVEGKSCINRCFYVELIRGRKEETEVLWTSNSIVVFCGTS
GTYGTGSWPDGADLNLMHI*

The sequence of the CA09 H1 antigen used here is:

(SEQ ID NO: 24)
MKAILVVLLYTFATANADTLCIGYHANNSTDTVDTVLEKNVTVTHSVNLL
EDKHNGKLCKLRGVAPLHLGKCNIAGWILGNPECESLSTASSWSYIVETP
SSDNGTCYPGDFIDYEELREQLSSVSSFERFEIFPKTSSWPNHDSNKGVT
AACPHAGAKSFYKNLIWLVKKGNSYPKLSKSYINDKGKEVLVLWGIHHPS
TSADQQSLYQNADAYVFVGSSRYSKKFKPEIAIRPKVRDREGRMNYYWTL
VEPGDKITFEATGNLWPRYAFAMERNAGSGIIISDTPVHDCNTTCQTPKG
AINTSLPFQNIHPITIGKCPKYVKSTKLRLATGLRNIPSIQSRGLFGAIA
GFIEGGWTGMVDGWYGYHHQNEQGSGYAADLKSTQNAIDEITNKVNSVIE
KMNTQFTAVGKEFNHLEKRIENLNKKVDDGFLDIWTYNAELLVLLENERT
LDYHDSNVKNLYEKVRSQLKNNAKEIGNGCFEFYHKCDNTCMESVKNGTY
DYPKYSEEAKLNREEIDGVKLESTRIYQILAIYSTVASSLVLVVSLGAIS
FWMCSNGSLQCRICI*

The sequence of the HA strain A/California/7/2009 (H1N1) (CA09) antigen mRNA open reading frame (ORF) used here is:

(SEQ ID NO: 8)
AUGAAAGCUAUCCUGGUCGUCUUGCUGUAUACUUUCGCCACUGCCAACGC
CGACACCCUGUGUAUCGGUUACCACGCGAACAACUCCACCGACACUGUGG
ACACCGUGCUCGAAAAGAACGUGACCGUGACUCAUUCUGUGAAUCUGCUC
GAGGACAAGCACAACGGAAAGUUGUGCAAGCUGCGCGGAGUGGCACCGCU
GCACCUUGGAAAGUGCAACAUUGCCGGAUGGAUCCUGGGAAACCCGGAGU
GCGAAAGCCUGAGCACCGCGUCCUCAUGGUCCUACAUCGUGGAAACCCCG
UCCUCUGACAACGGCACCUGUUACCCCGGCGAUUUCAUCGACUACGAAGA
ACUGCGGGAGCAGCUGUCCUCCGUGUCCUCGUUUGAACGCUUCGAGAUUU
UCCCUAAGACCUCCAGCUGGCCUAAUCACGAUAGCAACAAGGGCGUGACG
GCAGCCUGCCCGCACGCCGGAGCAAAGUCAUUCUACAAGAAUCUGAUUUG
GCUCGUGAAGAAAGGGAACUCAUACCCCAAGCUGUCCAAGUCGUACAUCA
ACGACAAGGGAAAGGAAGUGCUCGUGCUCUGGGGGAUCCACCACCCAUCC
ACCUCCGCCGACCAGCAGAGCCUGUACCAGAACGCCGAUGCUUACGUGUU
UGUGGGUUCCAGCCGGUACUCCAAGAAGUUCAAGCCUGAAAUCGCGAUCA
GGCCUAAAGUCCGGGACCGCGAGGGCCGCAUGAACUACUACUGGACUCUC
GUGGAGCCUGGAGACAAGAUCACCUUCGAGGCCACCGGAAAUCUCGUGGU
GCCACGCUACGCUUUCGCCAUGGAACGGAACGCCGGAAGCGGCAUCAUCA
UUAGCGAUACUCCUGUGCAUGACUGUAACACCACGUGCCAGACACCCAAG
GGCGCCAUCAACACCAGCCUGCCGUUUCAAAACAUCCAUCCCAUUACCAU
UGGGAAGUGCCCCAAAUACGUCAAGUCCACCAAGCUGAGGCUGGCGACCG
GACUGCGGAACAUUCCGAGCAUCCAGUCGAGAGGCCUGUUCGGUGCCAUC
GCGGGAUUCAUCGAGGGCGGCUGGACUGGAAUGGUGGACGGUUGGUACGG
GUAUCACCACCAAAACGAACAGGGAUCAGGCUACGCGGCCGAUUUGAAGU
CCACCCAGAACGCCAUUGAUGAAAUCACCAACAAGGUCAACUCCGUGAUU
GAGAAGAUGAAUACUCAAUUCACCGCCGUGGGCAAAGAAUUCAAUCACCU
GGAGAAGAGAAUAGAGAACCUGAACAAGAAGGUCGACGACGGGUUCCUCG
ACAUCUGGACCUAUAACGCCGAGUUGCUCGUGCUGCUGGAAAACGAACGG
ACCCUGGACUAUCACGACUCGAACGUGAAGAACCUGUACGAGAAAGUCCG
CUCGCAACUGAAGAACAACGCCAAGGAAAUCGGAAAUGGUUGCUUCGAGU
UCUACCAUAAGUGCGACAACACUUGCAUGGAGUCCGUGAAGAACGGCACU
UACGAUUACCCCAAGUACUCCGAAGAGGCUAAACUUAACCGGGAAGAGAU
CGAUGGCGUGAAGCUCGAGUCCACCAGAAUCUACCAGAUUCUCGCCAUCU
ACUCGACUGUGGCAUCGAGCCUCGUCCUUGUCGUGUCCCUGGGGGCCAUU
UCAUUCUGGAUGUGCUCCAACGGGUCCCUGCAGUGCCGGAUUUGCAUCUA
A

The sequence of the A/Michigan/45/2015 (Mich15) neuraminidase (NA) antigen mRNA open reading frame (ORF) used here is:

(SEQ ID NO: 9)
AUGAACCCAAACCAGAAAAUCAUCACGAUUGGCUCGAUUUGCAUGACCAU
UGGAAUGGCGAACCUUAUCCUCCAAAUUGGCAACAUUAUCUCGAUCUGGG
UCAGCCACUCGAUCCAGAUCGGCAACCAAUCCCAGAUUGAAACUUGCAAC
CAGAGCGUGAUUACUUACGAAAACAACACGUGGGUGAACCAGACUUACGU
CAAUAUUAGCAACACUAACUUCGCCGCUGGGCAGAGCGUCGUCAGCGUGA
AGCUCGCCGGAAAUUCCUCGCUCUGCCCCGUGUCCGGCUGGGCGAUCUAC
AGCAAGGAUAACAGCGUCCGGAUUGGUAGCAAGGGCGACGUUUUCGUGAU
CCGCGAACCCUUCAUAUCAUGCUCCCCGCUCGAAUGUCGCACGUUCUUCC
UGACCCAAGGCGCCCUGCUGAACGACAAGCACUCCAAUGGCACUAUCAAG
GAUCGGAGCCCUUACCGGACCUUGAUGUCCUGCCCUAUUGGAGAAGUGCC
UUCACCAUAUAACUCGCGCUUUGAAAGCGUGGCUUGGUCAGCCUCCGCCU
GCCAUGACGGGAUUAACUGGCUGACCAUUGGCAUAAGCGGCCCCGAUUCC
GGCGCCGUGGCCGUCCUGAAGUACAACGGGAUCAUCACCGACACCAUUAA
GUCCUGGCGCAACAACAUCCUGAGGACCCAGGAGUCCGAGUGCGCGUGCG
UGAACGGGUCCUGCUUUACCAUCAUGACCGACGGACCGUCCGACGGUCAA
GCCUCGUACAAGAUCUUCCGGAUCGAGAAAGGAAAGAUCAUCAAGAGCGU
GGAGAUGAAGGCCCCGAACUACCACUACGAGGAAUGUUCAUGCUAUCCCG
ACUCGUCCGAGAUUACUUGCGUGUGCCGCGACAAUUGGCACGGAUCCAAC
AGGCCGUGGGUCAGCUUCAACCAGAACCUUGAAUACCAGAUGGGAUACAU
UUGCAGCGGAGUGUUCGGGGACAACCCUCGCCCGAACGACAAGACCGGAU
CGUGUGGGCCCGUGUCCUCCAACGGCGCAAACGGCGUCAAGGGAUUUUCC
UUCAAAUACGGGAACGGGGUCUGGAUCGGACGGACCAAGAGCAUUUCAAG
CAGAAAGGGAUUCGAGAUGAUUUGGGACCCGAACGGCUGGACUGGUACCG
AUAACAAAUUCAGCAUCAAGCAGGACAUCGUGGGAAUUAACGAGUGGUCC
GGUUACUCCGGGAGCUUCGUGCAGCAUCCCGAACUCACUGGACUGGACUG
CAUUCGGCCGUGCUUUUGGGUGGAAUUGAUCCGGGGCAGACCUGAGGAGA
ACACGAUUUGGACCUCCGGCUCCUCGAUCUCGUUCUGCGGAGUGAACUCC
GACACCGUGGGAUGGUCCUGGCCCGACGGUGCAGAGCUGCCCUUCACCAU
UGAUAAGUAA

The sequence of the A/Singapore.INFIMH160019/2016 (Sing16; H3N2) HA hemagglutinin antigen mRNA open reading frame (ORF) used here is:

(SEQ ID NO: 10)
AUGAAAACCAUAAUCGCGCUCUCAUACAUACUUUGCCUGGUCUUUGCCCA
AAAGAUCCCUGGCAACGACAACUCAACCGCGACCCUUUGCCUCGGCCAUC
ACGCCGUGCCGAACGGCACUAUCGUCAAGACCAUCACAAACGACCGCAUC
GAAGUGACCAACGCGACUGAGCUAGUGCAGAACUCCAGCAUUGGAGAGAU
UUGCGAUUCUCCACACCAAAUCCUGGACGGAGAGAAUUGUACCUUGAUCG
ACGCGCUGCUGGGGGAUCCGCAGUGCGACGGAUUCCAGAACAAGAAAUGG
GACCUUUUCGUGGAACGGAGCAAGGCAUACUCGAAUUGCUACCCCUACGA
UGUGCCCGACUACGCCUCGCUGCGGUCCUUGGUCGCUUCCUCCGGGACCC
UGGAAUUCAAAAACGAGAGCUUUAAUUGGACCGGAGUGACCCAGAAUGGC
ACCUCGAGCGCCUGCAUUCGGGGCUCCUCCUCGAGCUUCUUCAGCCGCCU
GAACUGGCUCACUCACCUCAACUACACCUACCCGGCACUGAACGUGACCA
UGCCGAACAAGGAACAAUUCGACAAGCUCUACAUUUGGGGGGUGCAUCAC
CCGGGUACCGAUAAGGACCAGAUCUUCCUCUACGCCCAAUCCUCGGGCCG
GAUCACCGUGUCCACUAAGCGCUCGCAGCAGGCCGUGAUCCCGAACAUUG
GAAGCAGACCCCGCAUUCGCGACAUUCCAUCGAGGAUCUCGAUCUACUGG
ACGAUUGUCAAGCCUGGCGACAUCCUCCUCAUUAACUCCACCGGGAACCU
CAUCGCCCCUCGGGGUUAUUUCAAGAUCCGCAGCGGGAAGUCCUCCAUCA
UGAGAAGCGAUGCCCCCAUUGGAAAGUGCAAGUCCGAGUGUAUCACACCU
AACGGAAGCAUUCCCAAUGACAAGCCAUUCCAGAACGUGAACAGAAUUAC
CUACGGAGCUUGCCCUCGCUACGUCAAACAUUCGACCCUCAAGUUGGCGA
CUGGAAUGCGCAACGUGCCGGAGAAGCAAACCCGGGGGAUCUUCGGGGCU
AUCGCGGGAUUCAUCGAAAAUGGAUGGGAAGGAAUGGUCGAUGGUUGGUA
CGGUUUCAGACACCAGAACUCCGAGGGGCGGGGCCAGGCCGCAGACCUGA
AGUCCACUCAGGCCGCGAUUGACCAGAUCAACGGAAAGCUCAACAGACUC
AUUGGAAAGACCAACGAAAAGUUCCACCAAAUCGAAAAGGAAUUCUCCGA
AGUGGAGGGCCGGGUGCAAGACCUGGAGAAGUACGUGGAGGACACUAAGA
UCGACCUUUGGAGCUAUAACGCAGAACUCCUUGUGGCCCUGGAAAACCAG
CACACCAUCGACCUGACCGAUUCAGAGAUGAACAAGCUCUUUGAGAAAAC
UAAGAAGCAACUCCGGGAAAACGCUGAGGACAUGGGAAAUGGAUGCUUUA
AGAUCUACCACAAGUGCGACAACGCCUGCAUUGAGUCCAUACGGAACGAA
ACUUACGACCAUAACGUCUACCGGGAUGAAGCCCUGAACAACAGAUUCCA
GAUCAAGGGCGUGGAGCUGAAGUCCGGCUACAAAGAUUGGAUCCUGUGGA
UUUCCUUCGCGAUUUCAUGCUUCUUGCUCUGCGUGGCCCUCCUGGGAUUC
AUAAUGUGGGCCUGUCAGAAGGGCAACAUUAGGUGCAACAUAUGCAUAUA
A

The sequence of the Perth/16/2009 (H3N2) NA antigen mRNA open reading frame (ORF) used here is:

(SEQ ID NO: 11)
AUGAACCCUAACCAGAAGAUCAUCACAAUUGGAAGCGUGUCCCUGACCAU
UUCGACGAUUUGCUUCUUCAUGCAAAUCGCGAUCUUGAUUACCACCGUCA
CCCUGCAUUUCAAGCAAUACGAAUUCAACUCCCCGCCAAACAACCAAGUC
AUGCUCUGCGAGCCCACCAUCAUCGAACGCAACAUCACCGAGAUCGUGUA
CCUUACCAACACUACCAUCGAAAAGGAGAUUUGCCCCAAGUUGGCCGAAU
ACCGGAACUGGAGCAAGCCCCAGUGUGACAUCACGGGAUUUGCGCCAUUC
AGCAAGGAUAACUCGAUCAGACUUUCCGCCGGGGGCGACAUUUGGGUCAC
UCGGGAGCCUUACGUGAGCUGCGACCCGGACAAGUGCUACCAAUUCGCAC
UCGGACAGGGUACCACCCUGAACAACGUCCAUAGCAACAACACCGUGCGC
GAUAGAACCCCGUACCGCACCCUCCUCAUGAACGAACUGGGAGUGCCGUU
CCACUUGGGAACCAAACAAGUCUGCAUUGCAUGGUCCUCCUCCUCCUGCC
ACGACGGCAAAGCCUGGCUUCACGUUUGCAUCACCGGCGACGACAAGAAU
GCGACGGCCUCCUUCAUAUACAAUGGUAGACUCGUGGAUAGCGUGGUGUC
AUGGUCCAAGGAAAUUCUCAGGACUCAGGAGUCAGAGUGCGUGUGCAUCA
ACGGGACUUGCACUGUCGUGAUGACCGACGGAUCGGCCUCCGGAAAGGCC
GACACUAAGAUCCUCUUCAUCGAGGAGGGAAAGAUCGUGCACACUUCUAC
CCUGAGCGGCUCGGCUCAGCAUGUCGAAGAGUGCUCGUGCUACCCCCGGU
AUCCCGGGGUCCGCUGCGUGUGCCGGGACAAUUGGAAAGGCUCAAACCGC
CCCAUCGUGGACAUUAACAUCAAGGACCACUCCAUCGUGAGCUCCUACGU
AUGCAGCGGGCUGGUCGGGGAUACCCCGCGGAAGAACGAUUCCUCGUCCU
CCUCCCACUGCCUGGACCCUAACAACGAAGAGGGAGGCCACGGAGUGAAG
GGAUGGGCUUUUGACGAUGGCAACGACGUGUGGAUGGGCAGGACUAUUUC
CGAAAAGUCCCGGCUGGGAUACGAAACCUUCAAGGUCAUCGAGGGCUGGU
CCAACCCGAAGUCAAAGCUCCAGAUCAACCGCCAGGUCAUCGUGGAUAGG
GGCAAUAGAUCCGGCUACUCCGGGAUCUUCAGCGUGGAAGGGAAGUCCUG
CAUUAACCGAUGCUUCUACGUGGAACUCAUUCGGGGUCGGAAGGAGGAAA
CCGAAGUGCUGUGGACUUCGAACUCAAUCGUGGUGUUUUGUGGGACCUCC
GGAACUUACGGAACUGGGUCCUGGCCUGACGGUGCCGACAUCAACCUUAU
GCCGAUCUAA

The sequence of the A/Wisconsin/588/2019 antigen mRNA open reading frame (ORF) used here is:

(SEQ ID NO: 12)
AUGAAAGCCAUCCUUGUUGUCAUGCUGUACACAUUCACCACCGCAAAUGC
GGAUACCCUGUGUAUCGGCUACCACGCAAAUAAUUCCACCGACACCGUUG
AUACCGUCCUGGAAAAGAACGUGACAGUGACUCACAGCGUCAAUCUCCUU
GAGGAUAAACAUAAUGGCAAGCUGUGCAAGCUGAGAGGCGUGGCUCCCCU
GCAUCUGGGAAAGUGCAACAUCGCUGGUUGGAUCCUCGGGAACCCAGAGU
GUGAGUCCCUCUCAACCGCACGGUCUUGGUCAUACAUCGUGGAGACUAGC
AAUUCAGACAACGGCACAUGCUACCCCGGUGACUUCAUUAACUACGAGGA
GCUGAGAGAACAGCUGAGUUCCGUGUCAUCCUUCGAGAGAUUCGAAAUCU
UCCCCAAAACCUCCUCCUGGCCCAAUCAUGACUCCGACAAUGGAGUGACA
GCCGCUUGUCCCCACGCCGGUGCCAAGAGUUUCUAUAAGAACCUCAUCUG
GCUGGUGAAAAAGGGCAAGUCCUAUCCCAAAAUUAACCAGACCUACAUUA
ACGAUAAGGGGAAAGAAGUCCUGGUCCUGUGGGGGAUACACCACCCCCCU
ACCAUCGCCGACCAGCAGUCUCUGUAUCAGAACGCCGACGCCUACGUGUU
CGUGGGUACCAGCCGUUAUAGUAAAAAGUUCAAGCCAGAAAUUGCCACCA
GACCUAAGGUGCGCGACCAGGAGGGCCGCAUGAACUACUACUGGACCCUG
GUGGAACCUGGCGACAAGAUUACAUUCGAGGCCACUGGGAACCUGGUGGC
ACCCAGAUACGCCUUUACAAUGGAACGGGAUGCUGGGAGCGGAAUCAUUA
UCUCCGAUACCCCUGUCCACGACUGCAAUACUACCUGUCAGACCCCAGAA
GGCGCUAUCAAUACCUCUCUGCCUUUCCAAAACGUGCACCCUAUCACUAU
CGGGAAAUGUCCCAAGUAUGUGAAAAGCACCAAACUGCGCCUGGCAACCG
GUCUGAGAAAUGUGCCCUCCAUCCAGUCCCGCGGCUUGUUCGGUGCAAUC
GCUGGCUUUAUCGAGGGUGGCUGGACUGGAAUGGUCGAUGGCUGGUACGG
CUACCAUCACCAGAACGAGCAGGGGUCCGGGUAUGCUGCCGACCUGAAAA
GCACUCAGAACGCCAUCGAUAAAAUCACUAACAAGGUGAACUCCGUGAUC
GAAAAGAUGAAUACACAGUUCACAGCAGUUGGCAAGGAGUUCAACCACCU
GGAAAAACGGAUAGAGAACCUGAAUAAGAAAGUCGAUGAUGGCUUUCUGG
ACAUCUGGACUUACAAUGCCGAGCUGCUGGUGCUCCUGGAAAACGAGCGG
ACACUGGAUUAUCACGACUCAAACGUGAAGAACCUGUAUGAAAAGGUGCG
UAACCAGCUGAAAAACAACGCCAAGGAAAUCGGCAAUGGCUGUUUCGAAU
UUUACCACAAGUGUGAUAAUACCUGUAUGGAGAGCGUUAAGAACGGGACU
UACGACUACCCAAAAUACAGCGAGGAGGCCAAGCUGAACCGGGAGAAGAU
CGACGGCGUCAAACUCGACUCCACUAGAAUAUACCAGAUUCUCGCCAUCU
AUAGCACAGUGGCAUCAAGUCUCGUCCUGGUGGUGUCACUGGGAGCCAUC
AGCUUUUGGAUGUGCAGCAAUGGAUCCCUCCAGUGUAGGAUCUGCAUCUA
A

The sequence of the A/Tasmania/503/2020 antigen mRNA open reading frame (ORF) used here is:

(SEQ ID NO: 13)
AUGAAGACCAUCAUCGCUCUGUCCUACAUCCUGUGCCUGGUGUUUGCUCA
GAAAAUCCCCGGGAAUGACAAUUCCACUGCCACUCUCUGCCUGGGCCAUC
AUGCCGUGCCAAAUGGAACCAUUGUCAAGACUAUAACAAAUGACCGCAUC
GAAGUGACCAACGCUACCGAGCUGGUUCAGAACAGCAGUAUUGGAGAAAU
CUGCGAUUCCCCACACCAGAUACUGGAUGGCGGCAACUGCACCCUGAUCG
ACGCACUGCUGGGUGACCCUCAGUGCGACGGAUUUCAGAAUAAGGAGUGG
GACCUUUUCGUUGAGCGCAGCAGAGCCAAUAGCAACUGCUACCCGUACGA
CGUGCCGGAUUACGCCAGUCUUCGAAGCCUGGUCGCAUCCAGCGGGACAC
UGGAGUUUAAGAAUGAGUCCUUUAAUUGGACAGGCGUGAAGCAGAACGGG
ACUAGCAGCGCAUGCAUUCGGGGCAGUAGCUCAUCCUUCUUUAGCCGACU
GAACUGGCUGACCCACCUCAACUACACAUACCCCGCACUGAAUGUGACUA
UGCCAAACAAAGAACAGUUUGACAAACUGUACAUCUGGGGAGUGCACCAU
CCUAGCACAGACAAGGACCAGAUCAGCCUGUUUGCCCAGCCCAGCGGCAG
GAUUACCGUGUCCACAAAACGGUCACAGCAAGCCGUGAUCCCUAAUAUUG
GAUCCCGCCCCCGGAUAAGGGACAUCCCUAGUCGCAUCAGUAUCUACUGG
ACCAUCGUGAAGCCCGGAGAUAUCUUGCUCAUCAAUAGCACUGGCAACCU
CAUUGCCCCCAGGGGCUAUUUUAAGAUCAGAAGCGGCAAGUCCAGCAUUA
UGCGCAGCGACGCACCCAUUGGCAAGUGCAAGUCCGAGUGCAUCACUCCU
AAUGGGUCCAUCCCAAACGACAAGCCAUUCCAAAAUGUCAACAGAAUCAC
CUACGGGGCUUGCCCCCGCUACGUGAAGCAGAGUACACUGAAACUGGCCA
CCGGGAUGCGCAACGUGCCCGAGAAGCAAACUAGAGGCAUCUUUGGAGCU
AUCGCUGGCUUCAUUGAGAAUGGCUGGGAGGGUAUGGUGGACGGCUGGUA
CGGAUUCCGCCACCAGAAUAGCGAAGGCAGAGGCCAGGCAGCAGACUUGA
AGUCCACCCAGGCCGCCAUUGAUCAGAUCAACGGCAAACUGAAUCGGCUU
AUUGGAAAAACAAACGAGAAGUUCCAUCAGAUUGAGAAGGAGUUUAGCGA
GGUGGAGGGCCGCGUGCAGGAUCUGGAAAAGUACGUUGAAGACACCAAGA
UCGACCUGUGGUCAUACAAUGCAGAGCUGCUCGUUGCCCUGGAAAAUCAG
CACACAAUUGACCUUACAGACUCCGAAAUGAAUAAGCUCUUUGAAAAGAC
CAAGAAGCAGCUGCGCGAGAACGCCGAGGAUAUGGGGAACGGUUGUUUUA
AGAUCUACCACAAGUGUGACAACGCCUGCAUUGGGUCCAUCCGAAAUGAA
ACAUACGACCACAACGUGUAUAGAGAUGAGGCCCUGAACAACCGAUUCCA
GAUUAAGGGAGUCGAGCUGAAGAGUGGCUAUAAGGACUGGAUCCUGUGGA
UCUCAUUCGCCAUGUCAUGCUUCCUUCUGUGUAUUGCUCUGCUCGGCUUC
AUCAUGUGGGCUUGCCAGAAAGGCAAUAUCCGGUGCAACAUCUGCAUCUA
A

The sequence of the B/Washington/02/2019 antigen mRNA open reading frame (ORF) used here is:

(SEQ ID NO: 14)
AUGAAAGCAAUCAUAGUGCUGCUGAUGGUGGUGACUAGCAAUGCCGAUCG
GAUCUGCACCGGCAUCACUUCCAGUAACAGCCCUCAUGUGGUCAAAACCG
CCACACAGGGCGAGGUGAACGUGACCGGAGUGAUUCCACUGACAACUACA
CCAACGAAGAGUCACUUCGCCAACCUGAAGGGCACCGAAACACGAGGCAA
GCUCUGCCCCAAGUGUCUGAAUUGCACCGACCUGGACGUCGCUUUGGGCC
GCCCUAAAUGUACCGGCAAAAUACCUUCCGCCAGAGUGUCCAUCCUGCAC
GAGGUGCGCCCCGUGACCUCCGGGUGUUUUCCCAUAAUGCACGACCGCAC
UAAAAUCCGCCAGCUGCCCAAUCUUCUGAGGGGGUACGAACAUGUCAGGC
UGUCCACUCACAACGUGAUCAACGCAGAAGACGCCCCCGGAAGGCCUUAU
GAGAUUGGAACCAGUGGGUCCUGCCCAAACAUUACCAACGGCAACGGCUU
CUUCGCCACUAUGGCCUGGGCCGUGCCAAAGAACAAGACCGCCACCAACC
CCCUGACAAUUGAAGUCCCUUACAUCUGCACAGAGGGAGAGGAUCAGAUC
ACCGUGUGGGGGUUUCACUCUGAUAACGAAACUCAGAUGGCCAAGCUGUA
CGGGGAUUCUAAACCCCAGAAGUUCACCAGUAGCGCUAACGGGGUGACCA
CCCAUUAUGUGUCUCAGAUCGGAGGUUUCCCAAAUCAGACCGAGGACGGC
GGACUGCCCCAGUCUGGAAGGAUCGUAGUGGACUAUAUGGUGCAGAAGAG
UGGAAAAACCGGCACCAUUACCUAUCAGCGCGGCAUACUGCUGCCACAGA
AGGUGUGGUGUGCUUCCGGCAGGUCCAAGGUUAUCAAAGGGUCCCUCCCC
CUGAUCGGCGAAGCAGAUUGUCUGCACGAGAAGUACGGCGGACUGAAUAA
GAGCAAACCCUACUACACCGGAGAACACGCUAAGGCAAUUGGGAAUUGUC
CGAUCUGGGUGAAGACGCCCCUGAAACUGGCCAAUGGCACAAAAUACCGG
CCCCCCGCUAAGCUGCUGAAGGAACGGGGGUUCUUCGGCGCCAUAGCCGG
CUUUCUGGAGGGAGGCUGGGAGGGCAUGAUAGCCGGGUGGCACGGCUACA
CUUCCCAUGGGGCUCACGGGGUGGCUGUGGCCGCCGACCUGAAGUCUACG
CAGGAAGCUAUCAACAAAAUCACUAAGAACCUGAACAGCCUGUCGGAAUU
GGAGGUCAAGAAUCUGCAGCGGCUGAGCGGCGCCAUGGAUGAGCUGCACA
AUGAGAUCCUGGAGCUUGACGAGAAGGUCGAUGAUCUUCGGGCCGAUACA
AUUAGUAGCCAAAUUGAGUUGGCCGUGCUGCUCAGCAACGAAGGCAUAAU
CAACAGCGAGGACGAGCACCUCCUGGCUCUGGAGAGAAAGCUGAAGAAGA
UGCUCGGCCCUAGCGCAGUUGAGAUCGGAAACGGCUGCUUCGAAACCAAG
CACAAGUGCAACCAGACCUGCCUGGACAGGAUCGCGGCAGGAACAUUCGA
CGCUGGGGAAUUCAGCCUCCCCACCUUCGACAGCCUGAACAUCACAGCCG
CCAGUCUGAAUGAUGACGGACUGGAUAACCAUACCAUCCUGCUGUACUAC
UCUACCGCUGCUUCCUCCCUGGCCGUGACAUUGAUGAUCGCAAUCUUUGU
GGUUUAUAUGGUGAGCCGAGACAACGUCAGUUGCAGUAUCUGCCUUUAA

The sequence of the B/Phuket/3073/2013 antigen mRNA open reading frame (ORF) used here is:

(SEQ ID NO: 15)
AUGAAAGCCAUCAUUGUGCUGCUGAUGGUUGUUACAAGCAACGCCGACCG
CAUCUGCACCGGGAUUACAAGCAGCAAUAGCCCUCACGUGGUGAAGACAG
CAACACAGGGAGAGGUGAACGUGACCGGCGUGAUUCCACUGACAACCACC
CCAACUAAAUCUUACUUUGCAAACCUGAAAGGGACACGGACCAGAGGAAA
GCUGUGCCCUGAUUGCCUGAAUUGCACAGACCUGGACGUGGCCCUGGGCA
GACCAAUGUGCGUGGGCACUACACCAAGCGCCAAGGCCUCCAUCCUCCAU
GAGGUGCGGCCCGUGACUUCUGGAUGUUUCCCCAUUAUGCACGACAGAAC
CAAGAUUAGACAGCUGCCAAACCUGCUCCGCGGCUACGAGAAAAUUCGCC
UGUCUACACAGAAUGUGAUCGACGCCGAGAAGGCUCCAGGAGGACCAUAC
AGACUGGGGACUUCUGGCAGCUGCCCUAACGCCACCUCUAAGAUCGGGUU
CUUCGCAACCAUGGCUUGGGCCGUGCCUAAAGACAAUUACAAGAAUGCCA
CCAAUCCACUGACUGUCGAGGUGCCAUAUAUUUGCACAGAGGGGGAGGAC
CAGAUCACUGUGUGGGGCUUUCAUAGCGAUAAUAAGACUCAGAUGAAGUC
UCUCUACGGCGACUCUAACCCUCAGAAGUUCACCUCCUCUGCCAACGGGG
UGACAACACACUACGUGUCCCAGAUCGGGGACUUUCCUGACCAGACCGAG
GAUGGAGGACUGCCUCAGUCUGGACGCAUCGUGGUGGACUAUAUGAUGCA
GAAGCCUGGGAAGACCGGCACUAUCGUGUACCAGAGGGGCGUGCUGCUGC
CCCAAAAGGUGUGGUGUGCCUCCGGAAGAAGCAAAGUGAUUAAGGGAUCC
CUGCCUCUGAUUGGGGAGGCCGAUUGCCUGCAUGAAGAGUAUGGAGGGCU
GAACAAGUCCAAGCCAUACUAUACAGGAAAGCACGCAAAAGCCAUCGGCA
ACUGUCCCAUCUGGGUCAAAACUCCUCUGAAGCUGGCCAACGGCACCAAA
UACCGCCCUCCAGCCAAGCUGCUGAAAGAACGCGGAUUCUUCGGCGCCAU
UGCAGGGUUUCUGGAAGGAGGCUGGGAGGGCAUGAUUGCUGGAUGGCACG
GAUAUACCUCUCACGGCGCUCACGGGGUGGCCGUGGCCGCCGAUCUGAAG
UCCACACAGGAGGCAAUUAACAAGAUCACCAAGAAUCUGAAUUCACUGUC
CGAGCUCGAAGUGAAAAACCUGCAGCGCCUGUCCGGCGCCAUGGACGAGC
UGCACAAUGAAAUCCUGGAGCUGGACGAGAAGGUGGACGACCUGCGGGCU
GACACUAUCAGCAGCCAGAUCGAGCUGGCAGUGCUGCUGAGCAAUGAGGG
CAUCAUCAACUCAGAAGACGAACACCUCCUGGCACUGGAAAGGAAACUCA
AGAAGAUGCUGGGCCCCUCCGCAGUGGACAUUGGGAACGGCUGUUUCGAA
ACCAAGCAUAAGUGUAACCAGACUUGUCUGGAUAGGAUCGCAGCAGGAAC
CUUCAACGCCGGCGAAUUUUCUCUGCCAACAUUUGACUCCCUGAACAUCA
CAGCUGCAUCCCUGAACGACGACGGACUGGACAAUCACACCAUCCUGCUG
UACUACUCUACUGCCGCUAGCUCCCUGGCCGUGACCCUGAUGCUGGCCAU
CUUCAUCGUGUACAUGGUUUCCAGGGAUAACGUGUCUUGUAGCAUUUGCC
UGUAA

### Results

mRNA Antigen Preparation, Characterization, and Expression

mRNAs coding for the full-length codon-optimized HA and NA for the various influenza strains were synthesized enzymatically using unmodified ribonucleotides. All mRNA preparations had >95% of 5′ Cap1 and showed a single homogenous peak on capillary electrophoresis. mRNA-LNP formulations were prepared by mixing the various lipid components with mRNA under controlled conditions and at fixed ratios. All mRNA-LNPs exhibited >95% encapsulation with uniform hydrodynamic radius ranging from 95-105 nm and a poly dispersity index (PDI) of 0.060-0.136 as shown in Table 5.

TABLE 5
Attributes of LNP Formulations Used in Mouse Preclinical Testing

| LNP | Size (nm) | PDI | % Encapsulation |
|---|---|---|---|
| CA09 HA | 97.54 | 0.117 | 95.2 |
| Sing16 HA | 103.2 | 0.068 | 97.3 |
| Sing16 NA | 105.8 | 0.128 | 96.5 |
| Mich15 NA | 103.3 | 0.136 | 97.4 |

Cryo-electron microscopy (Cryo-TEM) of the CA09 HA mRNA-LNP images showed uniform spherical particles with a multi-lamellar inner core structure. The lamellarity of the solid core structure analyzed further with Fourier Transform, indicated a 3.7 nm periodicity between layers. The uniform morphology of the particles seen in the micrographs are indicative of homogenous LNP preparations with proper assembly of the LNPs.

Antigen expression was confirmed with flow cytometry by transiently transfecting human skeletal muscle cells (HskMCs) with the unencapsulated mRNA constructs of CA09 HA, Sing16 HA, Sing16 NA, or Mich15 NA, and stained with protein-specific antibodies for analysis. High levels of HA and NA expression from HskMCs were observed, confirming proper assembly and trafficking of native form HA trimers and NA tetramers upon expression in muscle cells. To study the subcellular localization of expressed HA and NA proteins, HeLa cells were transfected with bivalent H3N2 LNP and proteins were visualized by immunostaining and confocal microscopy. While NA signal indicated strong colocalization in ER (about 90%), HA was found to colocalize moderately (25%) with ER when permeabilized cells were stained with antibodies for corresponding proteins and Calnexin, an endoplasmic reticulum (ER) marker. This is consistent with the understanding that nascent NA and HA proteins are translocated to ER for assembly (Dou et al., Front Immunol. (2018) 9:1581).

The efficiency of delivery of mRNA by LNPs and selection of optimal formulation parameters was evaluated using reporter mRNA expression (Thess et al., Molecular Therapy (2015) 23(1):S55). A single dose of either 0.05, 0.1, 1, 5, μg of unmodified FF-LNP formulations was administered intramuscularly (IM) in mice. Luciferase activity, measured by average bioluminescence, indicated sustained expression from mRNA construct which peaked at 6 hours post injection and detectable beyond 72 hours at all doses (FIG. 11, panel (a)). The high-level mRNA-mediated protein expression was further verified with hEPO at a single 0.1 μg dose in mice and 10 μg in non-human primate (NHP). The study was intended to compare LNP, using standard LNP Dlin-MC3-DMA25 formulation as a control. Serum hEPO quantified by ELISA demonstrated maximum expression at 6 h with approximately 12-fold higher erythropoietin expressed with hEPO-LNP compared to hEPO-MC3 (FIG. 11, panel (c)). Both hEPO-LNP and hEPO-MC3 showed similar expression kinetics in NHPs, detectable from 6 hours to 72 hours (FIG. 11, panel (d)). The results confirmed the utility of the present LNP formulation for efficient delivery of mRNA for expression both in vitro and in vivo.

Immunogenicity of HA (H1, H3) and NA (N1, N2) mRNA-LNP in Mice

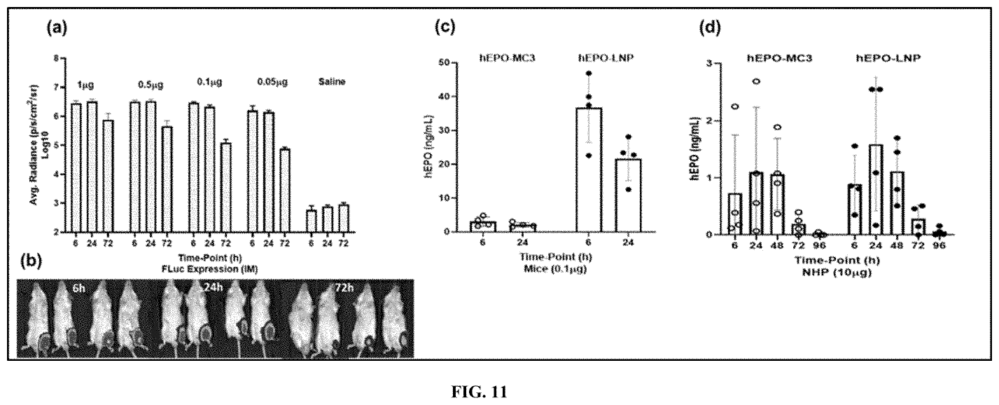

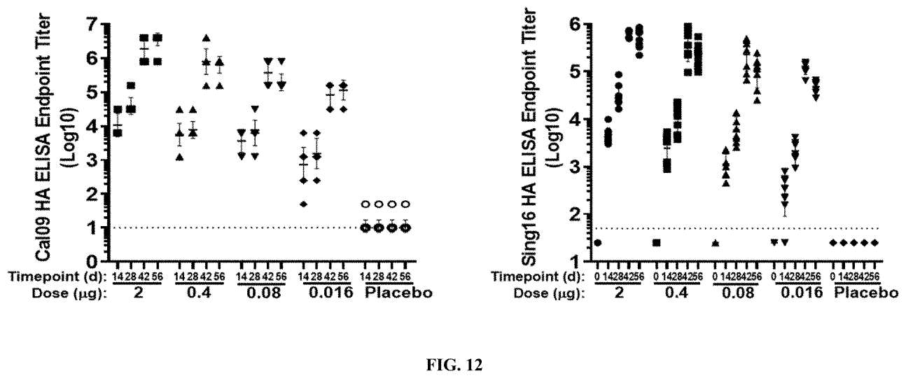

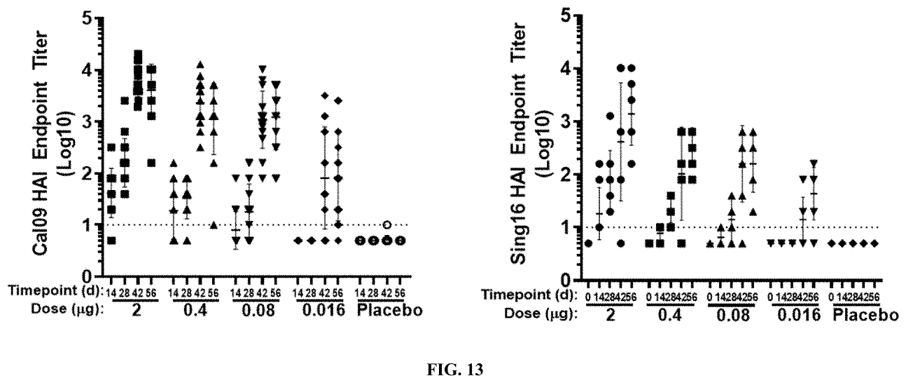

Natural history and vaccine studies have shown that antibodies to influenza HA and NA have antiviral function and both antigens are considered important for effective influenza vaccines (Krammer et al., Nat Rev Immunol. (2019) 19(6):383-97). Unmodified CA09 HA-LNP and Sing16 HA-LNP mRNA vaccines were evaluated in BALB/c mice (n=8) in a two-dose regimen at 2, 0.4, 0.08, or 0.016 μg mRNA-LNP administered at 4-week apart schedule. Recombinant HA (rHA) antigens of the same strain were used to evaluate the total IgG responses in ELISAs. HA-specific antibodies were detected in all groups after a single dose, but the titers peaked at day 42 after the second dose (FIG. 12). To measure functional antibodies, hemagglutination inhibition (HAI) response was evaluated against the homologous strains, CA09 and Sing16. Although the HAI titers after a first dose could be observed for the 2 μg dose of CA09-LNP and Sing16-LNP treatment groups with GMTs of 160 and GMT 70 at day 28 respectively, a more profound increase in HAI titers were observed after second dose. At day 42 GMT titers were 80 and 2200 for the 0.016 μg and 0.4 μg groups respectively in the CA09-HA-LNP and 14 and 100 for the 0.016 μg and 0.4 μg groups respectively in the Sing 16 HA-LNP groups (FIG. 13).

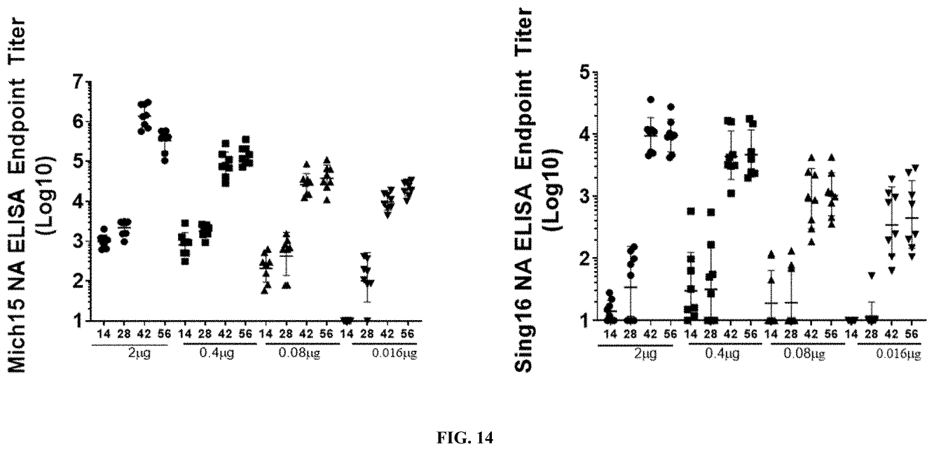

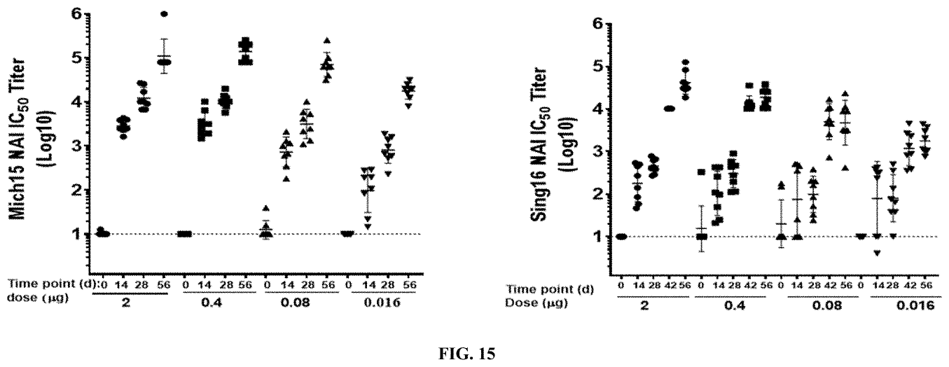

Similarly, for testing anti-NA responses, mice were immunized with 2, 0.4, 0.08, or 0.016 μg of Sing16 NA-LNP or Mich15 NA-LNP. ELISA with recombinant NA antigens were conducted to assess the total IgG responses induced by either Mich15 NA-LNP or Sing16 NA-LNP formulations. Animals developed high antibody binding responses after a single dose, with a marked increase in NA binding antibodies post second dose at day 42 (FIG. 14). Enzyme-linked lectin assay (ELLA) was used as a surrogate for functional antibody titers for Neuraminidase inhibition (NAI) activity against H6N1 or H6N2 chimeric viruses. Although two doses of the vaccine substantially increased the functional antibody response as compared to a single dose, encouraging NAI titers with GMTs 800 and GMT 60 were recorded at day 28 after a single dose even with low dose of 0.016 μg of Mich15 NA-LNP and Sing16 NA-LNP, respectively. At day 42, the GMT titers between the 0.4 μg and 0.016 μg, were 900 and 10200 respectively in the Sing16 NA-LNP group indicating a dose-dependent response with titers reaching above ULOQ in case of Mich15 NA-LNP (FIG. 15).

Protection from Viral Challenge in Mice

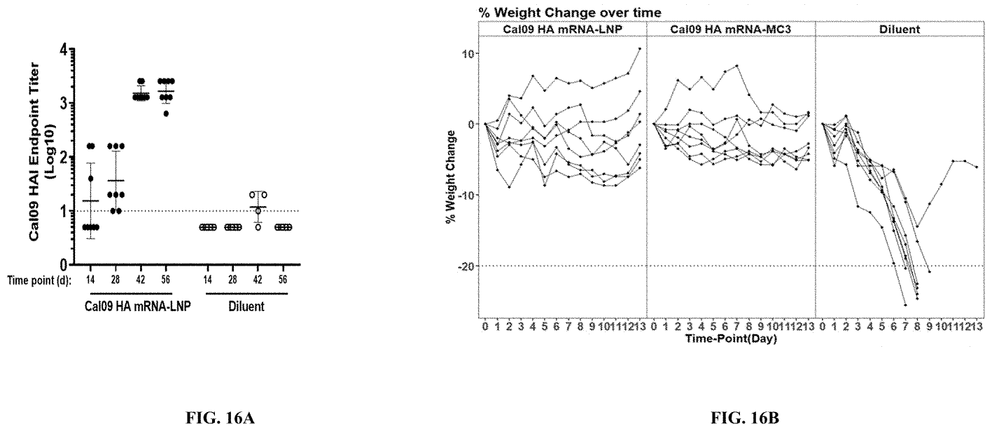

To test the efficacy of the mRNA vaccine in mouse influenza virus challenge model, we inoculated BALB/c mice with 0.4 μg of CA09 HA-LNP IM at week 0 and 4, along with a negative control group with two doses of LNP diluent buffer. HAI titers for vaccine group serum samples at study days 0, 14, 28, 42, 56, 92, and 107 demonstrated robust immune response with GMT of 1660 and 1:830 at day 56 and day 92 respectively (FIG. 16A). At day 93, all mice were challenged intranasally with Belgium09 virus, homologous to CA09, at four times the dose which can cause 50% lethal outcome (4×LD₅₀). All mice in the vaccine group survived the challenge with no mortality, and some mild morbidity marked by transient weight loss of less than 5% (FIG. 16B). However, those in the diluent control group suffered significant and rapid weight loss which led to high mortality rate (90%) by day 9. These results demonstrated high efficacy of HA-based MRT formulations in a lethal mouse influenza challenge model.

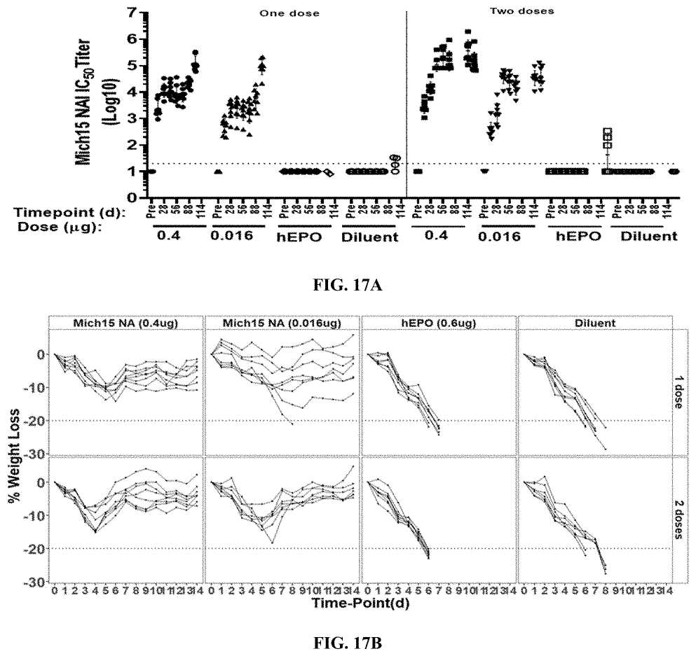

To assess protective efficacy of NA-based MRT vaccines, we conducted an analogous challenge experiment in BALB/c mice. Since the Mich15 NA-LNP vaccine elicited robust NAI titers after a single immunization in naïve mice (FIG. 16A), we evaluated one or two dosing regimens with administrations of 0.4 or 0.016 μg of Mich15 NA-LNPs over a 4-week interval. The control groups were vaccinated at the same regimens, receiving either 0.6 μg hEPO-LNP or diluent buffer. Robust NAI titers were observed after a single administration with GMTs of 14,000 NAI for 0.4 μg and 1,800 NAI for 0.016 μg of Mich15 NA-LNP recorded at day 28 (FIG. 17A). After the second immunization at day 42, NAI titers rose to 108,000 NAI for 0.4 μg and 37,000 NAI for 0.016 μg groups. After more than 12 weeks post vaccination regimens, all groups were challenged with 4×LD₅₀ of Belgium09 H1N1 virus. Individual weight changes from baseline over time by treatment groups are graphed in FIG. 17B. All mice in the two control groups suffered significant morbidity, and all animals had to be euthanized due to >20% weight loss by day 8 post-infection. Remarkably, all animals except one in the vaccine groups survived the challenge in the single dose 0.016 μg group, indicating high protective efficacy against death even after a single dose of as low as 0.016 ptg of Mich15 NA-LNP. The higher dose (0.4 μg) demonstrated overall higher protection, however, in contrast to HA-immunization, NA vaccination was not sufficient to protect against weight loss as vaccinated animals demonstrated median weight loss of 10% of initial body weight, consistent with observations reported for other NA vaccines. Body weight recoveries were observed for vaccinated groups resulting in an average final weight change of 2.7% at the low dose and 4.8% weight gain for the higher dose, as compared to baseline. Overall, the results demonstrated that a single low-dose MRT NA-LNP vaccination can elicit functional antibodies measurable for blocking influenza NA activity and sufficient to confer protection against lethal challenge in mice.

Immunogenicity of HA (H3) mRNA-LNP in NHP

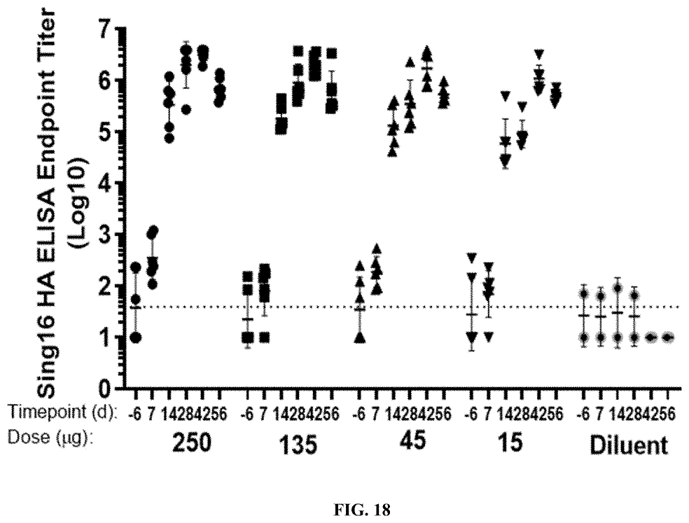

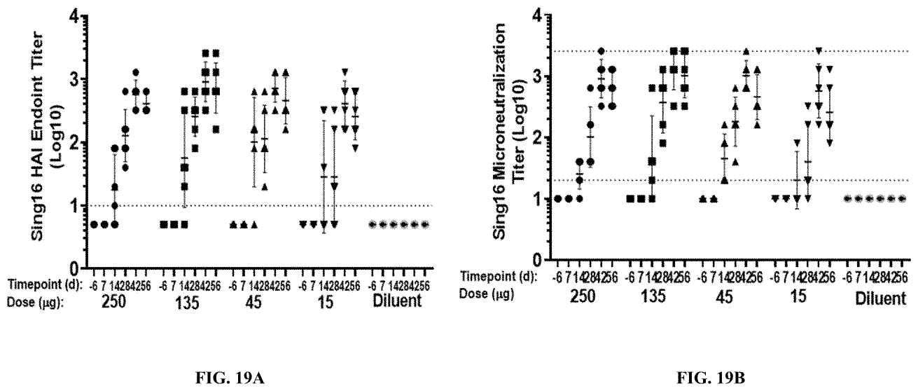

To evaluate immunogenicity of the mRNA-LNP in NHP, a dose range study covering 15, 45, 135, and 250 μg of Sing16 HA-LNP was performed in NHPs. After the first immunization, all vaccinated NHPs developed antibodies reactive to recombinant HA protein as noted in ELISA (FIG. 18). Further boosting of titers was observed post second dose. Surprisingly, the 15 μg dose induced only 1.8-fold lower ELISA titers than the 135 μg dose level (95% CI 1.0, 3.6), suggesting a dose saturation close to 15 μg level. Robust HAI antibodies were induced in all dose groups on day 42 and GMTs recorded were 400 for 15 μg, 700 for 45 μg, 900 for 135 μg and 570 for 250 μg. At day 42, the fold increase in GMT titers with 95% CI was 2.2-fold (1.0; 5.0) between the 135 μg and 15 μg and was 1.3-fold (0.6; 2.8) between the 135 μg and 45 μg treatment groups indicating that despite the observed trend towards higher titers with increasing dose, the difference between groups was minimal (FIG. 19A). The neutralization potency assessed by microneutralization (MN) assay (FIG. 19B) showed a better trend for dose effect with GMTs on D28 of 40 for 15 μg, 180 for 45 μg, 300 and for 135 μg.

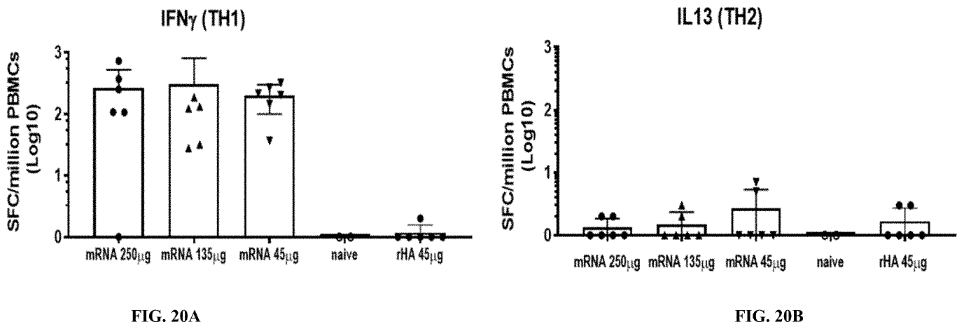

Since T cells have been shown effective in reducing viral load and limiting disease severity in animal models (Rimmelzwaan et al., Vaccine (2008) 26(4):D41-D44; Sridhar et al., Nat Med. (2013) 19(10):1305-12; Sridhar et al., Front Immunol. (2016) 7:195), we evaluated recall T cells in the NHPs vaccinated with 45, 135, 250 μg of Sing16 HA-LNP or with 45 μg of recombinant HA. PBMCs collected at day 42 were evaluated in IFN-γ (Th1 cytokine) and IL-13 (Th2 cytokine) ELISPOT assay with recall stimulation with pooled overlapping peptides spanning the entire sequence of the Sing16 HA. All vaccinated animals except one in 250 μg group developed IFN-γ secreting cells, ranging from 28 to 1328 spot-forming cells (SFC) per million PBMCs (FIG. 20A). Notably, a dose-response was not observed, and the lower and higher dose level groups of animals showed comparable frequencies of IFN-γ secreting cells. In contrast, all animals in the control group immunized with the recombinant Sing16 HA protein demonstrated absence of IFN-γ producing cells. The presence of IL-13 cytokine secreting cells was either not detected or very low in all the groups tested (FIG. 20B). The data suggest that Sing16 HA-LNP induced strong Th1-biased cellular responses in NHPs, comparable to that seen with MRT5500 (Kalnin et al., supra), a SARS-CoV-2 vaccine currently under development.

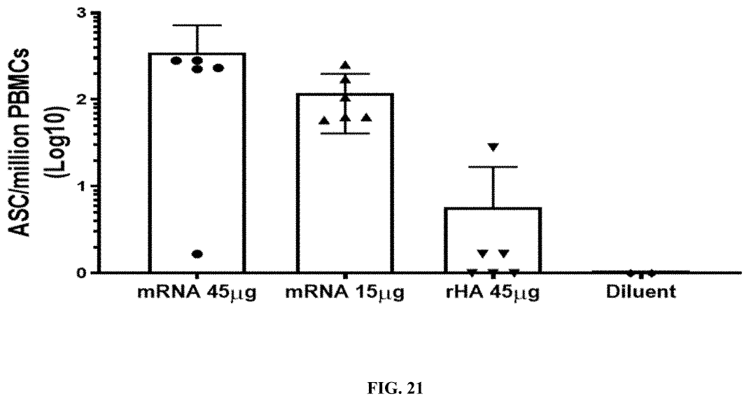

To investigate frequency of memory B cells (MBCs) in NHPs after immunization with Sing16 HA-LNP, an ELISPOT assay was developed to quantify antigen-specific MBCs as a readout of humoral immune memory. On day 180, PBMCs were collected from the NHPs immunized with 45 μg or 15 μg of the Sing16 HA mRNA-LNP formulations or with a recombinant HA as a comparator at a 45 μg dose. A 4-day polyclonal stimulation of PBMCs that is optimized to drive memory B cells to antibody secreting cells (ASC) was performed, and the stimulated PBMCs were plated in an antigen-specific ELISPOT where the frequency of antigen-specific ASCs could be determined. Antigen-specific memory B cells were then quantified as a percentage of total IgG+ memory B cells. Antigen-specific memory B cells were detected in all animals and their frequency was ranging from 1 to 5% for the 45 ug dose group and 0.3 to 1.5% for the 15 μg dose group. In the rHA immunized animals, the memory B cell responses appeared to be markedly lower as antigen-specific memory B cells were undetectable in five out of six animals (FIG. 21). We conclude that Sing16 HA-LNP, like other mRNA vaccines, elicits a population of anti-HA specific memory B cells that promise to prolong immunity (Lindgren et al., Front Immunol. (2019) 10:614).

Multivalent Influenza Virus Antigens

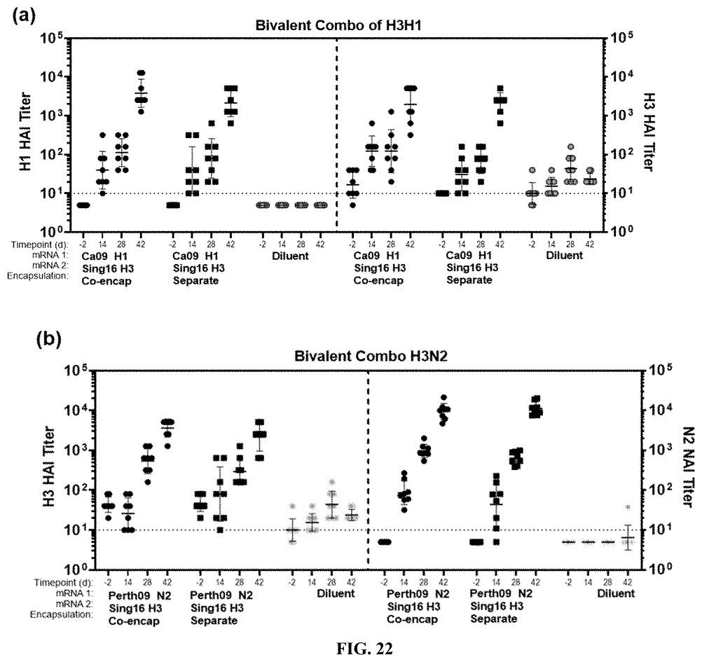

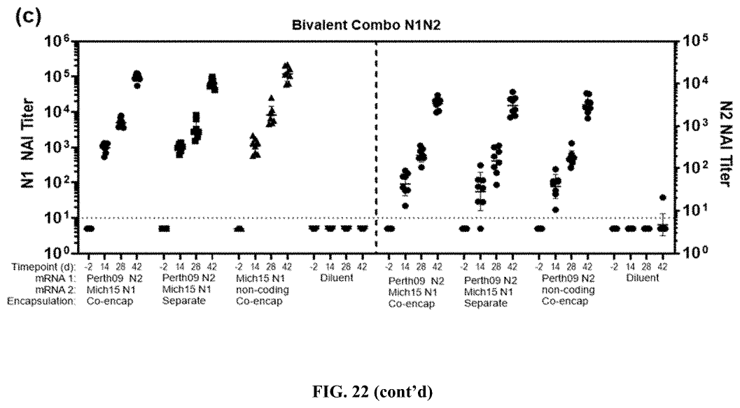

An advantage of mRNA-LNP platform is the flexibility of LNP encapsulation for multiple mRNA antigen constructs. However, this potential needs to be tested to address the concern of antigenic interference. To explore the combinations of influenza antigens, co-encapsulated HA and NA mRNA were formulated in LNPs as bivalent formulations containing 0.2 μg each of mRNA in an H3H1, H3N2, or N1N2 combination or with the monovalent containing 0.2 μg of each corresponding antigen. These formulations were administered in mice to determine any antigenic interference on immunogenicity by comparing the functional titers of the individual antigen in bivalent vs. monovalent formulations (FIG. 22, panels (a)-(c) and Table 6).

TABLE 6
Frequency of Antigen-Specific Memory B Cells
in NHPs Vaccinated with H3 mRNA-LNP Vaccine

| | | PBMCs/ | Spot # of | | Spot # of | % of |
|---|---|---|---|---|---|---|
| | | well of | Ag-Specific | PBMCs/ | Total IgG/ | Ag-Specific |
| | | Ag-Specific | IgG/million | Well of | million | IgG to |
| Animal group | Animal ID | IgG | PBMCs | Total IgG | PBMCs | Total IgG |
| H3 mRNA-LNP | 1 | 3 × 10⁵ | 1082 | 5 × 10³ | 21700 | 5.0 |
| (45 μg) | 2 | 3 × 10⁵ | 232 | 5 × 10³ | 6100 | 3.8 |
| | 3 | 3 × 10⁵ | 282 | 5 × 10³ | 11700 | 2.4 |
| | 4 | 3 × 10⁵ | 2 | 5 × 10³ | 100 | 2.0 |
| | 5 | 3 × 10⁵ | 283 | 5 × 10³ | 8700 | 3.3 |
| | 6 | 3 × 10⁵ | 225 | 5 × 10³ | 22800 | 1.0 |
| H3 mRNA-LNP | 1 | 3 × 10⁵ | 63 | 5 × 10³ | 21600 | 0.3 |
| (15 μg) | 2 | 3 × 10⁵ | 58 | 5 × 10³ | 11300 | 0.5 |
| | 3 | 3 × 10⁵ | 253 | 5 × 10³ | 17300 | 1.5 |
| | 4 | 3 × 10⁵ | 173 | 5 × 10³ | 17300 | 1.0 |
| | 5 | 3 × 10⁵ | 63 | 5 × 10³ | 9300 | 0.7 |
| | 6 | 3 × 10⁵ | 107 | 5 × 10³ | 19300 | 0.6 |
| rHA | 1 | 3 × 10⁵ | 2 | 5 × 10³ | 19800 | 0.0 |
| (45 μg) | 2 | 3 × 10⁵ | 28 | 5 × 10³ | 14300 | 0.2 |
| | 3 | 3 × 10⁵ | 2 | 5 × 10³ | 17000 | 0.0 |
| | 4 | 3 × 10⁵ | 0 | 5 × 10³ | 7900 | 0.0 |
| | 5 | 3 × 10⁵ | 0 | 5 × 10³ | 21600 | 0.0 |
| | 6 | 3 × 10⁵ | 0 | 5 × 10³ | 14600 | 0.0 |
| Diluent | 1 | 3 × 10⁵ | 0 | 5 × 10³ | 30900 | 0.0 |
| | 2 | 3 × 10⁵ | 0 | 5 × 10³ | 7100 | 0.0 |

In the H1H3 combo, between the co-encapsulated and separately administered vaccines no statistically significant difference (p=0.2584) irrespective of the time points was seen for HAI titers and no significant difference (p=0.8389) at D42 was seen for H3 titers. In the case of H3N2 combo, the NA component of the vaccine elicited high neutralizing antibodies in combination with the HA component demonstrating lack of HA dominance. Between the co-encapsulated and separately administered vaccines no statistically significant difference (p=0.2960) irrespective of the time points was seen for H3 titers and no significant difference (p=0.0904) at D42 was seen for N2 titers. Likewise, the N1N2 combo was not statistically significantly different (p=0.3899) for N2. N1 titers at day 42 for co-encapsulated and separately administered vaccines were above limit of quantification. Combination of N2N1, H3H1, or H3N2 thus generated antibody titers equivalent to individual LNPs separately formulated.

We further explored quadrivalent formulations of co-encapsulated H1, N1, H3, and/or N2 mRNA. These formulations were tested in NHPs in total 10 μg composed of 2.5 μg each of influenza antigen mRNA and filling amount of noncoding mRNA (nc mRNA) if needed in combinations, resulting in quadrivalent (H1N1H3N2), bivalent (H1N1 or H3N2), or monovalent (H1, H3, N1, or N2) LNPs (Table 7).

TABLE 7
Bivalent Combination of Influenza Virus in Mouse Study

| | | | | | mRNA | | | | | |
|---|---|---|---|---|---|---|---|---|---|---|
| | | | | | dose | | CA09 | Sing16 | Mich15 | Perth09 |
| Group | N | mRNA1 | mRNA2 | LNP | (μg) | Description | HAI | HAI | NAI | NAI |
| 1 | 8 | Sing16 | Perth09 | Yes | 0.2, 0.2 | Coformulated | | x | | x |
| 2 | 8 | H3 | N2 | | | Separate | | x | | x |
| 3 | 8 | CA09 | Sing16 | | | Coformulated | x | x | | |
| 4 | 8 | H1 | H3 | | | Separate | x | x | | |
| 5 | 8 | Mich15 | Perth09 | | | Coformulated | | | x | x |
| 6 | 8 | N1 | N2 | | | Separate | | | x | x |
| 7 | 8 | Diluent | — | — | 0 | single | x | x | x | x |

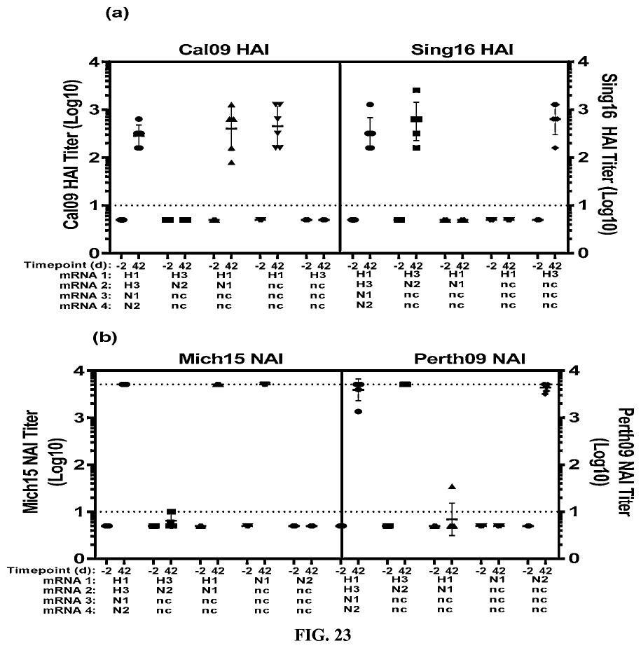

HAI titers to H1 or H3, or NAI titers to N1 or N2 were compared between the monovalent formulations vs. bivalent or quadrivalent formulations (FIG. 23). On day 42, the HAI titers to H1 of the quadrivalent group were comparable when analyzed with that of the H1 monovalent group (p=0.9054, t-test, unpaired, two-tailed) or H1N1 bivalent group (p=0.8002). Similarly, the H3 HAI titers of the quadrivalent group was comparable when analyzed with that of the H3 monovalent group (p=0.2504) or H3N2 bivalent group (p=0.5894). The NAI titers to N1 were almost identical in groups of animals vaccinated with N1 monovalent mRNA or H1N1 bivalent mRNA or the quadrivalent H1N1H3N2 mRNA formulations. Likewise, there was no difference in N2 NAI titers between the N2 monovalent mRNA (p=0.8485) or H3N2 bivalent mRNA (0.4545) with the quadrivalent H1N1H3N2 mRNA formulations.

Overall, these findings indicate that co-encapsulated or combination multivalent vaccines of HA/NA mRNA-LNPs at this dose level could efficiently deliver all four antigens without any concern for antigenic interference and all antigens were as immunogenic as in the formulation when these antigens were delivered singularly.

### Example 7: Additional LNP Formulations

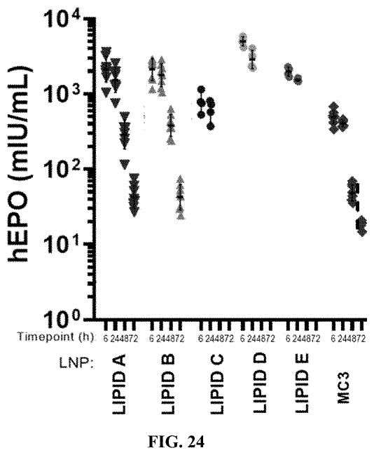

Additional LNP formulations for mRNA vaccines were prepared, designated Lipid C (containing cationic lipid GL-HEPES-E3-E10-DS-3-E18-1), Lipid D (containing cationic lipid GL-HEPES-E3-E12-DS-4-E10), and Lipid E (containing cationic lipid GL-HEPES-E3-E12-DS-3-E14). Human Erythropoietin (hEPO) mRNA was used as a test mRNA. Expression of hEPO was measured by ELISA from samples taken from mice injected with the LNPs. Samples were taken 6 hours, 24 hours, 48 hours, and 72 hours after injection. As show in FIG. 24, hEPO expression was consistently higher at all time points with LNP formulations Lipid A, Lipid B, Lipid C, Lipid D, and Lipid E, compared to a control LNP formulation containing cationic lipid MC3.

Table 8 below summarizes the results relative to a control LNP containing the MC3 cationic lipid.

TABLE 8
Levels of hEPO from LNP formulations Lipid A-E relative to MC3.

| | Fold higher hEPO | |
|---|---|---|
| | at 6 hours | |
| LNP Formulation | (compared to MC3) | STDEV |
| Lipid A | 10.35 | 4.15 |
| Lipid B | 5.62 | 1.34 |
| Lipid D | 7.78 | 2.79 |
| Lipid E | 6.17 | 1.57 |

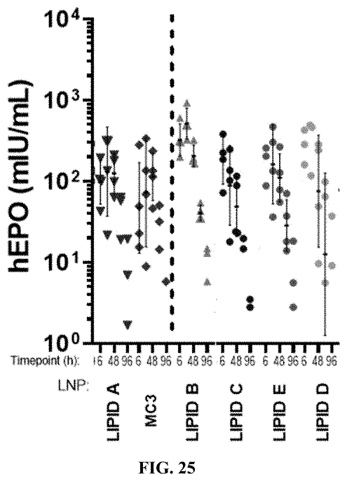

The same hEPO mRNA-LNP formulations were next tested in non-human primates (NHPs). Samples were taken at 6 hours, 48 hours, and 96 hours after injection. As shown in FIG. 25, each LNP formulation produced levels of hEPO comparable to the MC3 control formulation.

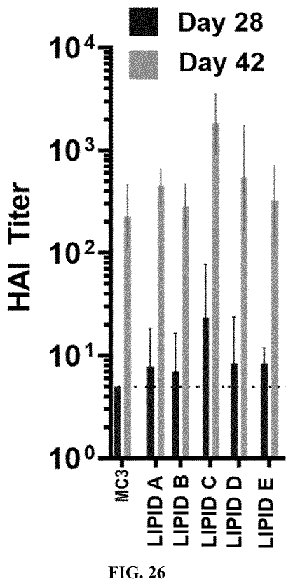

Influenza HA-encoding mRNA-LNP formulations were also tested in NHPs. NHPs were administered the LNP formulations at 10 μg via intramuscular injection and samples were taken at say 28 and day 42 post injection. HAI titers were measured as described above. As shown in FIG. 26, each LNP formulation produced HAI titers comparable to or higher than the MC3 control formulation.

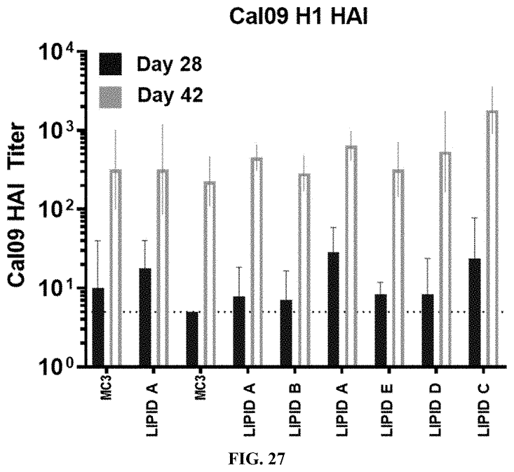

The same experiment as shown in FIG. 26 was performed while measuring HAI titers with the Cal09 H1 influenza antigen. As shown in FIG. 27, each LNP formulation produced HAI titers comparable to or higher than the MC3 control formulation.

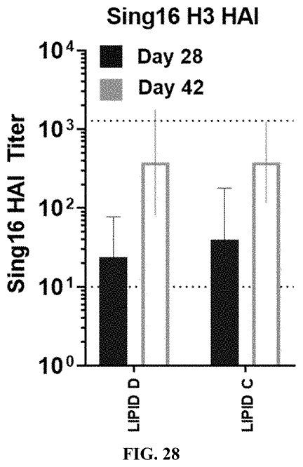

As shown in FIG. 28, HAI titers with the Sing16 H3 antigen were elevated for LNP formulations Lipid C and Lipid D.

### Example 8: Further Studies on Quadrivalent or Octavalent Influenza Vaccine LNP Formulations

HAI titers and NAI titers were measured from mice administered various multivalent LNP-influenza mRNA vaccines. HAI titers were measured against influenza strains A/Michigan/45/2015, A/SINGAPORE/INFIMH160019/2016, B/Maryland/15/2016 BX69A, and B/Phuket/3073/2013. NAI titers were measured against influenza strains A/Michigan/45/2015, A/SINGAPORE/INFIMH160019/2016, B/Colorado/06/201, and B/Phuket/3073/2013.

The HAI titers and NAI titers were compared against mice receiving mono- or quadrivalent HA or NA mRNA vaccines.

Mice were injected with a prime vaccine on Day 0 and a booster vaccine of the same dosage on Day 21. Blood was collected on Days 1, 20, 22, and 35. For monovalent compositions containing mRNA encoding HA or NA antigens, mRNA encoding each of the following individually was used: H1, H3, HA from a B/Victoria lineage, and HA from a B/Yamagata lineage (specifically from strains A/Michigan/45/2015; A/Singapore/Infimh160019/2016; B/Maryland/15/2016; and B/Phuket/3037/2013). Quadrivalent vaccine compositions containing mRNA encoding each of N1, N2, NA from a B/Victoria lineage and NA from a B/Yamagata lineage, and each of H1, H3, HA from a B/Victoria lineage and HA from a B/Yamagata lineage (specifically from strains A/Michigan/45/2015; A/Singapore/Infimh160019/2016; B/Colorado/06/2017; and B/Phuket/3037/2013) were also prepared. Finally, an octavalent vaccine composition containing mRNA encoding each of H1, H3, HA from a B/Victoria lineage, HA from a B/Yamagata lineage, each of N1, N2, NA from a B/Victoria lineage and NA from a B/Yamagata lineage (specifically from strains A/Michigan/45/2015; A/Singapore/Infimh160019/2016; B/Colorado/06/2017; and B/Phuket/3037/2013) was prepared and administered as an octavalent vaccine. Each mRNA for all compositions was added in an amount of 0.4 μg/strain. For each group, n=6 mice.

An overview of each experimental group is recited below in Table 9.

TABLE 9
Overview of experimental groups for multivalent influenza vaccines in mice

| | | | | Prime (D 0)/ | | |
|---|---|---|---|---|---|---|
| Group | | Prime (D 0)/ | Dose mRNA NA | boost (D 21) - HA (together | Dose rHA | Adjuvant |
| # | N | boost (D 21) - NA mRNA | (μg per strain) | with NA) | (μg per strain) | (rHA) |
| 1 | 6 | LNP diluent | — | — | — | — |
| 3 | 6 | NA mRNA-LNP (N2 Perth) | 0.4 | — | — | — |
| 4 | 6 | NA mRNA-LNP (N1) | 0.4 | — | — | — |
| 5 | 6 | NA mRNA-LNP (N2) | 0.4 | — | — | — |
| 6 | 6 | NA mRNA-LNP (NV) | 0.4 | — | — | — |
| 7 | 6 | NA mRNA-LNP (NY) | 0.4 | — | — | — |
| 8 | 6 | NA mRNA-LNP (N1, | 0.4 | — | — | — |
| | | N2, BV, BY) | | | | |
| 9 | 6 | — | — | HA mRNA-LNP (H1) | 0.4 | — |
| 10 | 6 | — | — | HA mRNA-LNP (H3) | 0.4 | — |
| 11 | 6 | — | — | HA mRNA-LNP (BV) | 0.4 | — |
| 12 | 6 | — | — | HA mRNA-LNP (BY) | 0.4 | — |
| 13 | 6 | — | — | HA mRNA-LNP (H1, | 0.4 | — |
| | | | | H3, BV, BY) | | |
| 14 | 6 | NA mRNA-LNP (N1, | 0.4 | HA mRNA-LNP (H1, | 0.4 | — |
| | | N2, BV, BY) | | H3, BV, BY) | | |

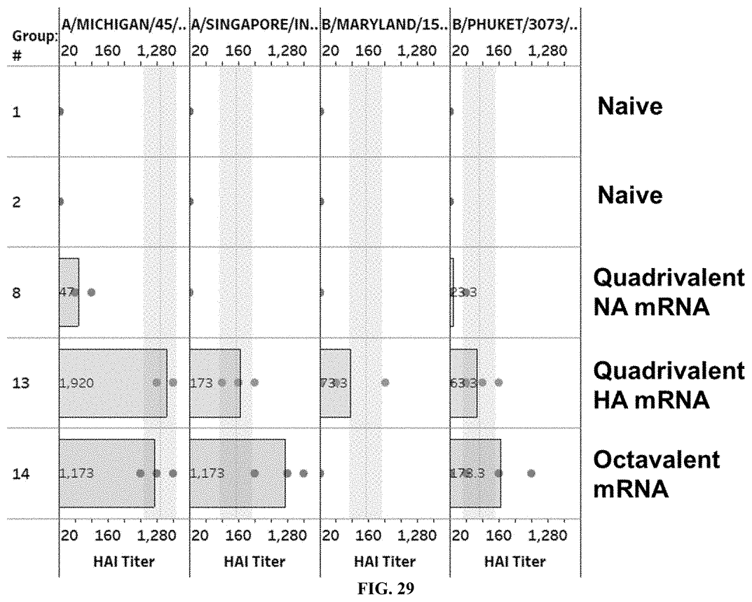

As shown in FIG. 29, octavalent mRNA-LNP formulations led to HAI titers within 4-fold of the quadrivalent for 3 out of 4 influenza strains.

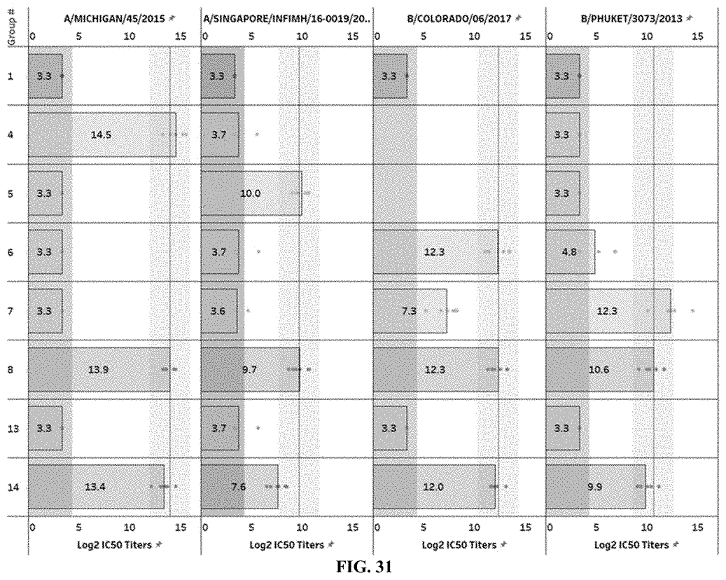

An overview of the NAI titer results for each of the groups above is shown in FIG. 31. The octavalent mRNA-LNP formulations led to NAI titers comparable to the quadrivalent mRNA-LNP formulations.

Thus, the data demonstrate that an octavalent vaccine was capable of inducing robust HA and NA immune responses and that the presence of the immunodominant HA from four different influenza strains does not appear to suppress or interfere with the anti-NA immune response.

High content imaging-based neutralization test (HINT) titers for HA and NAI titers were additionally measured from ferrets administered various multivalent LNP-influenza mRNA vaccines. The HINT assay is described in further detail in Jorquera et al. (Scientific Reports. 9: 2676. 2019), incorporated herein by reference. HINT titers were measured against influenza strains A/Michigan/45/2015, A/SINGAPORE/INFIMH160019/2016, B/IOWA/06/2017, and B/Phuket/3073/2013. NAI titers were measured against influenza strains A/Michigan/45/2015, A/SINGAPORE/INFIMH160019/2016, B/Colorado/06/201, and B/Phuket/3073/2013.

Ferrets used to assess multivalent vaccine immunogenicity were vaccinated twice 21 days apart with (1) a mixture of four mRNAs encoding NA antigens (N1, N2, BvNA, and ByNA), (2) a mixture of four mRNAs encoding HA antigens (H1, H3, BvHA, and ByHA), or (3) a mixture of four mRNAs encoding NA antigens (N1, N2, BvNA, and ByNA) and four mRNAs encoding HA antigens (H1, H3, BvHA, and ByHA), as shown below in Table 12. Each HA includes HA from one of the following four strains: A/Michigan/45/2015 (H1); A/Singapore/Infimh-16-0019/2016 (H3); B/Iowa/06/2017 (B/Victoria lineage); and B/Phuket/3073/2013 (B/Yamagata lineage). All antigens were administered at a 1:1 ratio.

An overview of each experimental group is recited below in Table 10.

All ferrets were bled under sedation (isoflurane) at baseline, one day before or just before booster, at booster vaccination, and two weeks after challenge as required. Sera samples (stored at −20° C. until required) were tested by ELLA to assess NAI activity. Additionally, the hemagglutinin inhibition assay (HAI) was undertaken to assess antibody responses to

TABLE 10
Overview of experimental groups for
multivalent influenza vaccines in ferrets

| Group | | Prime (D0)/ | Prime (D0)/ | Dose (μg | |
|---|---|---|---|---|---|
| # | N | boost (D21)-NA | boost (D21)-HA | per strain) | Adjuvant |
| 1 | 6 | PBS | PBS | 0 | — |
| 11 | 6 | NA mRNA-LNP | — | 1 | — |
| | | (N1, N2, BV, BY) | | | |
| 12 | 6 | NA mRNA-LNP | — | 15 | — |
| | | (N1, N2, BV, BY) | | | |
| 13 | 6 | — | HA mRNA-LNP | 1 | — |
| | | | (H1, H3, BV, BY) | | |
| 14 | 6 | — | HA mRNA-LNP | 15 | — |
| | | | (H1, H3, BV, BY) | | |
| 15 | 6 | NA mRNA-LNP | HA mRNA-LNP | 1 | — |
| | | (N1, N2, BV, BY) | (H1, H3, BV, BY) | | |
| 16 | 6 | NA mRNA-LNP | HA mRNA-LNP | 15 | — |
| | | (N1, N2, BV, BY) | (H1, H3, BV, BY) | | |

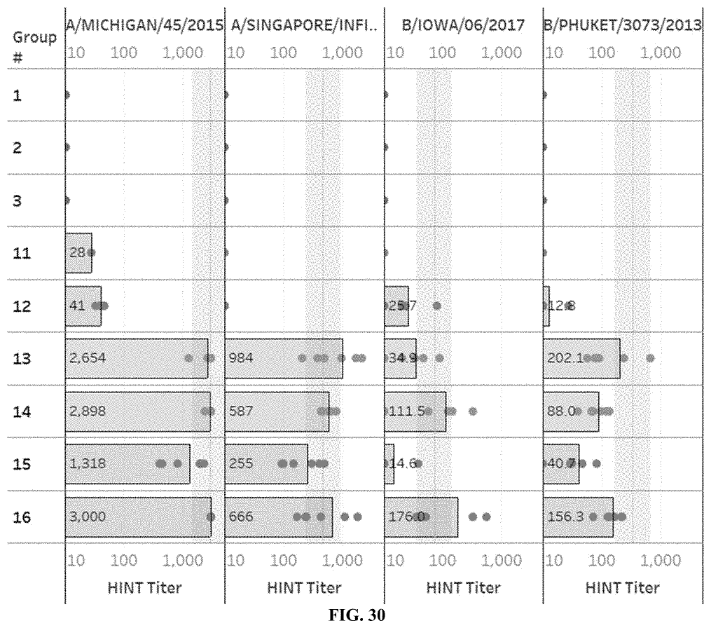

An overview of the HINT results for each of the groups above is shown in FIG. 30. The octavalent mRNA-LNP formulations led to HINT titers comparable to the quadrivalent mRNA-LNP formulations.

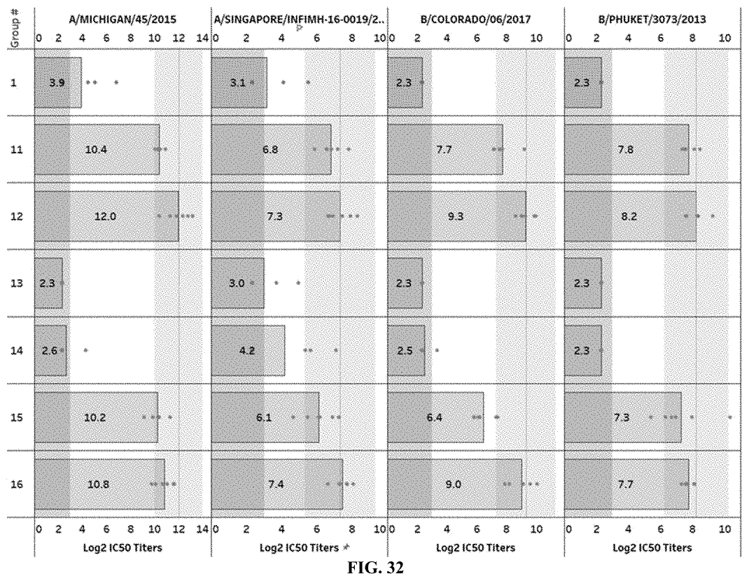

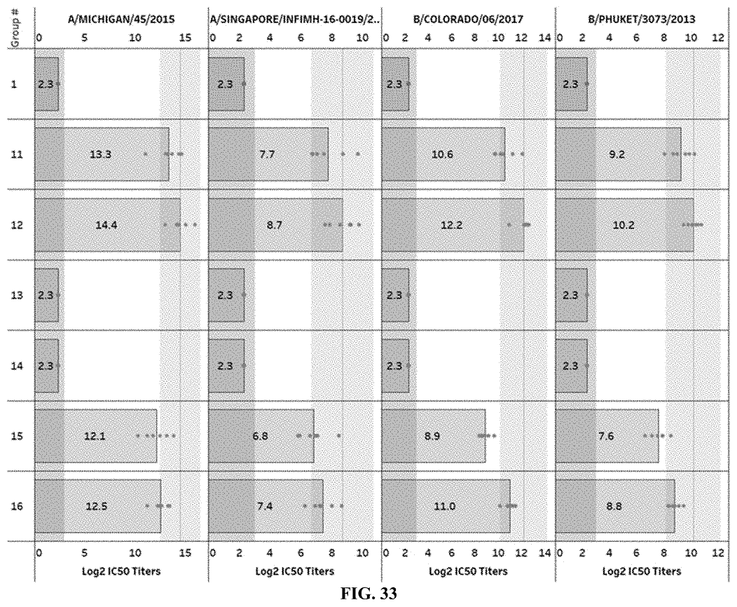

An overview of the NAI titer results for each of the groups above is shown in FIG. 32 (day 20) and FIG. 33 (day 42). The octavalent mRNA-LNP formulations led to NAI titers comparable to the quadrivalent mRNA-LNP formulations. This was true from the day 20 and day 42 samples.

1. An influenza vaccine composition, comprising eight messenger RNA (mRNA), each mRNA comprising an open reading frame (ORF) encoding a different influenza antigen.

2. The influenza vaccine composition of claim 1, wherein the composition comprises eight mRNA encoding (i) one or more hemagglutinin (HA) antigens, (ii) one or more neuraminidase (NA) antigens, or (iii) at least one HA antigen and at least one NA antigen.

3. The influenza vaccine composition of claim 1, wherein the composition comprises one or more mRNA encoding antigens of influenza A, B and/or C viruses, optionally wherein:
the HA antigens of influenza A viruses are selected from subtypes H1, H2, H3, H4, H5, H6, H7, H8, H9, H10, H11, H12, H13, H14, H15, H16, H17, and H18;
the NA antigens of influenza A viruses are selected from subtypes N1, N2, N3, N4, N5, N6, N7, N8, N9, N10, and N11; and/or
the HA and NA antigens of influenza B viruses are from the influenza B/Yamagata lineage or the influenza B/Victoria lineage.

4. The influenza vaccine composition of claim 1, wherein the antigens are HA and/or NA antigens of influenza A and influenza B viruses.

5-7. (canceled)

8. The influenza vaccine composition of claim 2, wherein the HA antigen and NA antigen is selected from the group consisting of H1N1, H3N2, H2N2, H5N1, H7N9, H7N7, H1N2, H9N2, H7N2, H7N3, H5N2, and H10N7 subtypes and/or B/Yamagata and B/Victoria lineages.

9. The influenza vaccine composition of claim 1, wherein the composition comprises one mRNA encoding an H3 HA antigen, one mRNA encoding an H1 HA antigen, one mRNA encoding an HA antigen from the Influenza B/Yamagata lineage, and one mRNA encoding an HA antigen from the Influenza B/Victoria lineage.

10. The influenza vaccine composition of claim 1, wherein the composition comprises one mRNA encoding an H3 HA antigen, one mRNA encoding an N2 NA antigen, one mRNA encoding an H1 HA antigen, one mRNA encoding an N1 NA antigen, one mRNA encoding an HA antigen from the Influenza B/Yamagata lineage, one mRNA encoding an NA antigen from the Influenza B/Yamagata lineage, one mRNA encoding an HA antigen from the Influenza B/Victoria lineage, and one mRNA encoding an NA antigen from the Influenza B/Victoria lineage.

11. (canceled)

12. The influenza vaccine composition of claim 1, wherein the mRNA molecule comprises at least one 5′ untranslated region (5′ UTR), at least one 3′ untranslated region (3′ UTR), and at least one polyadenylation (poly(A)) sequence, optionally wherein the ORF is codon optimized.

13. The influenza vaccine composition of claim 1, wherein the mRNA comprises at least one chemical modification, optionally wherein:
at least 20%, at least 30%, at least 40%, at least 50%, at least 60%, at least 70%, at least 80%, at least 85%, at least 90%, at least 95%, or 100% of the uracil nucleotides in the mRNA and/or in the ORF are chemically modified;
the chemical modification is selected from the group consisting of pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine; and/or
the chemical modification is selected from the group consisting of pseudouridine, N1-methylpseudouridine, 5-methylcytosine, 5-methoxyuridine, and a combination thereof.

14-18. (canceled)

19. The influenza vaccine composition of claim 1, wherein the mRNA is formulated in a lipid nanoparticle (LNP), optionally wherein the LNP comprises at least one cationic lipid.

20. (canceled)

21. The influenza vaccine composition of claim 19, wherein:
the cationic lipid is biodegradable or not biodegradable; and/or
the cationic lipid is cleavable or not cleavable.

22-24. (canceled)

25. The influenza vaccine composition of claim 19, wherein the cationic lipid is selected from the group consisting of OF-02, cKK-E10, GL-HEPES-E3-E10-DS-3-E18-1, GL-HEPES-E3-E12-DS-4-E10, and GL-HEPES-E3-E12-DS-3-E14.

26. The influenza vaccine composition of claim 19, wherein the LNP further comprises a polyethylene glycol (PEG) conjugated (PEGylated) lipid, a cholesterol-based lipid, and a helper lipid.

27. The influenza vaccine composition of claim 19, wherein the LNP comprises:
a cationic lipid at a molar ratio of 35% to 55%;
a polyethylene glycol (PEG) conjugated (PEGylated) lipid at a molar ratio of 0.25% to 2.75%;
a cholesterol-based lipid at a molar ratio of 20% to 45%; and
a helper lipid at a molar ratio of 5% to 35%,
wherein all of the molar ratios are relative to the total lipid content of the LNP.

28. The influenza vaccine composition of claim 27, wherein the LNP comprises:
a cationic lipid at a molar ratio of 40%;
a PEGylated lipid at a molar ratio of 1.5%;
a cholesterol-based lipid at a molar ratio of 28.5%; and
a helper lipid at a molar ratio of 30%.

29. The influenza vaccine composition of claim 26, wherein:
the PEGylated lipid is dimyristoyl-PEG2000 (DMG-PEG2000) or 2-[(polyethylene glycol)-2000]-N,N-ditetradecylacetamide (ALC-0159);
the cholesterol-based lipid is cholesterol; and/or
the helper lipid is 1,2-dioleoyl-SN-glycero-3-phosphoethanolamine (DOPE) or 1,2-distearoyl-sn-glycero-3-phosphocholine (DSPC).

30. (canceled)

31. (canceled)

32. The influenza vaccine composition of claim 19, wherein the LNP comprises:
a cationic lipid is selected from the group consisting of OF-02, cKK-E10, GL-HEPES-E3-E10-DS-3-E18-1, GL-HEPES-E3-E12-DS-4-E10, and GL-HEPES-E3-E12-DS-3-E14, at a molar ratio of 40%;
DMG-PEG2000 at a molar ratio of 1.5%;
cholesterol at a molar ratio of 28.5%; and
DOPE at a molar ratio of 30%.

33. The influenza vaccine composition of claim 19, wherein the LNP has an average diameter of 30 nm to 200 nm or an average diameter of 80 nm to 150 nm.

34. (canceled)

35. The influenza vaccine composition of claim 19, comprising between 1 mg/mL to 10 mg/mL of the LNP.

36. The influenza vaccine composition of claim 19, wherein the LNP comprises between 1 and 20 mRNA molecules, optionally wherein the LNP comprises 5-10 or 6-8 mRNA molecules.

37. (canceled)

38. The influenza vaccine composition of claim 19, wherein the LNP comprises two or more mRNA, wherein each mRNA encodes a different influenza antigen.

39. The influenza vaccine composition of claim 19, wherein the composition comprises eight LNPs, wherein each LNP comprises an mRNA encoding a different influenza antigen.

40. The influenza vaccine composition of claim 1, wherein the composition is formulated for intramuscular injection, optionally wherein the composition comprises a phosphate-buffer saline.

41. (canceled)

42. A method of eliciting an immune response in a subject in need thereof, comprising administering to the subject, optionally intramuscularly, intranasally, intravenously, subcutaneously, or intradermally, a prophylactically effective amount of an influenza vaccine composition comprising eight messenger RNA (mRNA), each mRNA comprising an open reading frame (ORF) encoding a different influenza antigen.

43. A method of preventing influenza infections or reducing one or more symptoms of influenza infections, comprising administering to the subject, optionally intramuscularly, intranasally, intravenously, subcutaneously, or intradermally, a prophylactically effective amount of the influenza vaccine composition of claim 1, optionally wherein the influenza vaccine composition elicits an immune response against one or more seasonal and/or pandemic influenza strains.

44. (canceled)

45. The method of claim 42, comprising:
administering to the subject one or more doses of the influenza vaccine composition, each dose comprising about 1 μg to about 250 μg of mRNA, optionally each dose comprising about 2.5, 5, 15, 45, or 135 μg of mRNA; and/or
administering to the subject two doses of the influenza vaccine composition with an interval of 2-6, optionally 4, weeks.

46-49. (canceled)

50. A kit comprising a container comprising a single-use or multi-use dosage of the composition of claim 1, optionally wherein the container is a vial or a pre-filled syringe or injector.

51. The influenza vaccine composition of claim 1, wherein the influenza antigens comprise an influenza virus HA antigen and/or an influenza virus NA antigen having a molecular sequence identified or designed from a machine learning model.

52. An influenza vaccine composition, comprising eight messenger RNA (mRNA), each mRNA comprising an open reading frame (ORF) encoding a different influenza antigen,
wherein the mRNA is formulated in a lipid nanoparticle (LNP),
wherein the LNP comprises:
a cationic lipid selected from the group consisting of OF-02, cKK-E10, GL-HEPES-E3-E10-DS-3-E18-1, GL-HEPES-E3-E12-DS-4-E10, and GL-HEPES-E3-E12-DS-3-E14, at a molar ratio of 35% to 55%;
a polyethylene glycol (PEG) conjugated (PEGylated) lipid at a molar ratio of 0.25% to 2.75%;
a cholesterol-based lipid at a molar ratio of 20% to 45%; and
a helper lipid at a molar ratio of 5% to 35%,
wherein all of the molar ratios are relative to the total lipid content of the LNP.

53. An influenza vaccine composition, comprising eight messenger RNA (mRNA), each mRNA comprising an open reading frame (ORF) encoding a different influenza antigen,
wherein the composition comprises one mRNA encoding an H3 HA antigen, one mRNA encoding an N2 NA antigen, one mRNA encoding an H1 HA antigen, one mRNA encoding an N1 NA antigen, one mRNA encoding an HA antigen from the Influenza B/Yamagata lineage, one mRNA encoding an NA antigen from the Influenza B/Yamagata lineage, one mRNA encoding an HA antigen from the Influenza B/Victoria lineage, and one mRNA encoding an NA antigen from the Influenza B/Victoria lineage.


SEQUENCE LISTING (25 sequences)

SEQ ID NO: 1 - protein, 566 aa, Artificial Sequence
  H3 sub type of Influenza virus hemagglutinin
MKTIIALSYILCLVFAQKIPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFKNESFNWTGVTQNGTSSACIRGSSSSFFSRLNWLTHLNYTYPALNVTMPNKEQFDKLYIWGVHHPGTDKDQIFLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKHSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIESIRNETYDHNVYRDEALNNRFQIKGVELKSGYKDWILWISFAISCFLLCVALLGFIMWACQKGNIRCNICI

SEQ ID NO: 2 - DNA, 21 bp, Artificial Sequence
  MRT10279
aggtgcaacatatgcatataa

SEQ ID NO: 3 - DNA, 21 bp, Artificial Sequence
  H3_WT
agatgcaacatttgcatttga

SEQ ID NO: 4 - protein, 469 aa, Artificial Sequence
  Perth09 N2 antigen
MNPNQKIITIGSVSLTISTICFFMQIAILITTVTLHFKQYEFNSPPNNQVMLCEPTIIERNITEIVYLTNTTIEKEICPKLAEYRNWSKPQCDITGFAPFSKDNSIRLSAGGDIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGRLVDSVVSWSKEILRTQESECVCINGTCTVVMTDGSASGKADTKILFIEEGKIVHTSTLSGSAQHVEECSCYPRYPGVRCVCRDNWKGSNRPIVDINIKDHSIVSSYVCSGLVGDTPRKNDSSSSSHCLDPNNEEGGHGVKGWAFDDGNDVWMGRTISEKSRLGYETFKVIEGWSNPKSKLQINRQVIVDRGNRSGYSGIFSVEGKSCINRCFYVELIRGRKEETEVLWTSNSIVVFCGTSGTYGTGSWPDGADINLMPI

SEQ ID NO: 5 - protein, 469 aa, Artificial Sequence
  Mich15 N1 antigen
MNPNQKIITIGSICMTIGMANLILQIGNIISIWVSHSIQIGNQSQIETCNQSVITYENNTWVNQTYVNISNTNFAAGQSVVSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 6 - protein, 566 aa, Artificial Sequence; identical to SEQ ID NO: 1
  Sing16 H3 antigen

SEQ ID NO: 7 - protein, 469 aa, Artificial Sequence
  Sing16 N2 antigen
MNPNQKIITIGSVSLTISTICFFMQIAILITTVTLHFKQYEFNSPPNNQVMLCEPTIIERNITEIVYLTNTTIEKEICPKPAEYRNWSKPQCGITGFAPFSKDNSIRLSAGGDIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGRLIDSVVSWSKDILRTQESECVCINGTCTVVMTDGNATGKADTKILFIEEGKIVHTSKLSGSAQHVEECSCYPRYPGVRCVCRDNWKGSNRPIVDINIKDHSIVSSYVCSGLVGDTPRKNDSSSSSHCLNPNNEEGGHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGADLNLMHI

SEQ ID NO: 8 - RNA, 1701 nt, Artificial Sequence
  HA strain A/California/7/2009 (H1N1) (CA09)
augaaagcuauccuggucgucuugcuguauacuuucgccacugccaacgccgacacccuguguaucgguuaccacgcgaacaacuccaccgacacuguggacaccgugcucgaaaagaacgugaccgugacucauucugugaaucugcucgaggacaagcacaacggaaaguugugcaagcugcgcggaguggcaccgcugcaccuuggaaagugcaacauugccggauggauccugggaaacccggagugcgaaagccugagcaccgcguccucaugguccuacaucguggaaaccccguccucugacaacggcaccuguuaccccggcgauuucaucgacuacgaagaacugcgggagcagcuguccuccguguccucguuugaacgcuucgagauuuucccuaagaccuccagcuggccuaaucacgauagcaacaagggcgugacggcagccugcccgcacgccggagcaaagucauucuacaagaaucugauuuggcucgugaagaaagggaacucauaccccaagcuguccaagucguacaucaacgacaagggaaaggaagugcucgugcucugggggauccaccacccauccaccuccgccgaccagcagagccuguaccagaacgccgaugcuuacguguuuguggguuccagccgguacuccaagaaguucaagccugaaaucgcgaucaggccuaaaguccgggaccgcgagggccgcaugaacuacuacuggacucucguggagccuggagacaagaucaccuucgaggccaccggaaaucucguggugccacgcuacgcuuucgccauggaacggaacgccggaagcggcaucaucauuagcgauacuccugugcaugacuguaacaccacgugccagacacccaagggcgccaucaacaccagccugccguuucaaaacauccaucccauuaccauugggaagugccccaaauacgucaaguccaccaagcugaggcuggcgaccggacugcggaacauuccgagcauccagucgagaggccuguucggugccaucgcgggauucaucgagggcggcuggacuggaaugguggacgguugguacggguaucaccaccaaaacgaacagggaucaggcuacgcggccgauuugaaguccacccagaacgccauugaugaaaucaccaacaaggucaacuccgugauugagaagaugaauacucaauucaccgccgugggcaaagaauucaaucaccuggagaagagaauagagaaccugaacaagaaggucgacgacggguuccucgacaucuggaccuauaacgccgaguugcucgugcugcuggaaaacgaacggacccuggacuaucacgacucgaacgugaagaaccuguacgagaaaguccgcucgcaacugaagaacaacgccaaggaaaucggaaaugguugcuucgaguucuaccauaagugcgacaacacuugcauggaguccgugaagaacggcacuuacgauuaccccaaguacuccgaagaggcuaaacuuaaccgggaagagaucgauggcgugaagcucgaguccaccagaaucuaccagauucucgccaucuacucgacuguggcaucgagccucguccuugucgugucccugggggccauuucauucuggaugugcuccaacgggucccugcagugccggauuugcaucuaa

SEQ ID NO: 9 - RNA, 1410 nt, Artificial Sequence
  A/Michigan/45/2015 (Mich15) neuraminidase
augaacccaaaccagaaaaucaucacgauuggcucgauuugcaugaccauuggaauggcgaaccuuauccuccaaauuggcaacauuaucucgaucugggucagccacucgauccagaucggcaaccaaucccagauugaaacuugcaaccagagcgugauuacuuacgaaaacaacacgugggugaaccagacuuacgucaauauuagcaacacuaacuucgccgcugggcagagcgucgucagcgugaagcucgccggaaauuccucgcucugccccguguccggcugggcgaucuacagcaaggauaacagcguccggauugguagcaagggcgacguuuucgugauccgcgaacccuucauaucaugcuccccgcucgaaugucgcacguucuuccugacccaaggcgcccugcugaacgacaagcacuccaauggcacuaucaaggaucggagcccuuaccggaccuugauguccugcccuauuggagaagugccuucaccauauaacucgcgcuuugaaagcguggcuuggucagccuccgccugccaugacgggauuaacuggcugaccauuggcauaagcggccccgauuccggcgccguggccguccugaaguacaacgggaucaucaccgacaccauuaaguccuggcgcaacaacauccugaggacccaggaguccgagugcgcgugcgugaacggguccugcuuuaccaucaugaccgacggaccguccgacggucaagccucguacaagaucuuccggaucgagaaaggaaagaucaucaagagcguggagaugaaggccccgaacuaccacuacgaggaauguucaugcuaucccgacucguccgagauuacuugcgugugccgcgacaauuggcacggauccaacaggccgugggucagcuucaaccagaaccuugaauaccagaugggauacauuugcagcggaguguucggggacaacccucgcccgaacgacaagaccggaucgugugggcccguguccuccaacggcgcaaacggcgucaagggauuuuccuucaaauacgggaacggggucuggaucggacggaccaagagcauuucaagcagaaagggauucgagaugauuugggacccgaacggcuggacugguaccgauaacaaauucagcaucaagcaggacaucgugggaauuaacgagugguccgguuacuccgggagcuucgugcagcaucccgaacucacuggacuggacugcauucggccgugcuuuuggguggaauugauccggggcagaccugaggagaacacgauuuggaccuccggcuccucgaucucguucugcggagugaacuccgacaccgugggaugguccuggcccgacggugcagagcugcccuucaccauugauaaguaa

SEQ ID NO: 10 - RNA, 1701 nt, Artificial Sequence
  A/Singapore.INFIMH160019/2016 (Sing16; H3N2)
augaaaaccauaaucgcgcucucauacauacuuugccuggucuuugcccaaaagaucccuggcaacgacaacucaaccgcgacccuuugccucggccaucacgccgugccgaacggcacuaucgucaagaccaucacaaacgaccgcaucgaagugaccaacgcgacugagcuagugcagaacuccagcauuggagagauuugcgauucuccacaccaaauccuggacggagagaauuguaccuugaucgacgcgcugcugggggauccgcagugcgacggauuccagaacaagaaaugggaccuuuucguggaacggagcaaggcauacucgaauugcuaccccuacgaugugcccgacuacgccucgcugcgguccuuggucgcuuccuccgggacccuggaauucaaaaacgagagcuuuaauuggaccggagugacccagaauggcaccucgagcgccugcauucggggcuccuccucgagcuucuucagccgccugaacuggcucacucaccucaacuacaccuacccggcacugaacgugaccaugccgaacaaggaacaauucgacaagcucuacauuuggggggugcaucacccggguaccgauaaggaccagaucuuccucuacgcccaauccucgggccggaucaccguguccacuaagcgcucgcagcaggccgugaucccgaacauuggaagcagaccccgcauucgcgacauuccaucgaggaucucgaucuacuggacgauugucaagccuggcgacauccuccucauuaacuccaccgggaaccucaucgccccucgggguuauuucaagauccgcagcgggaaguccuccaucaugagaagcgaugcccccauuggaaagugcaaguccgaguguaucacaccuaacggaagcauucccaaugacaagccauuccagaacgugaacagaauuaccuacggagcuugcccucgcuacgucaaacauucgacccucaaguuggcgacuggaaugcgcaacgugccggagaagcaaacccgggggaucuucggggcuaucgcgggauucaucgaaaauggaugggaaggaauggucgaugguugguacgguuucagacaccagaacuccgaggggcggggccaggccgcagaccugaaguccacucaggccgcgauugaccagaucaacggaaagcucaacagacucauuggaaagaccaacgaaaaguuccaccaaaucgaaaaggaauucuccgaaguggagggccgggugcaagaccuggagaaguacguggaggacacuaagaucgaccuuuggagcuauaacgcagaacuccuuguggcccuggaaaaccagcacaccaucgaccugaccgauucagagaugaacaagcucuuugagaaaacuaagaagcaacuccgggaaaacgcugaggacaugggaaauggaugcuuuaagaucuaccacaagugcgacaacgccugcauugaguccauacggaacgaaacuuacgaccauaacgucuaccgggaugaagcccugaacaacagauuccagaucaagggcguggagcugaaguccggcuacaaagauuggauccuguggauuuccuucgcgauuucaugcuucuugcucugcguggcccuccugggauucauaaugugggccugucagaagggcaacauuaggugcaacauaugcauauaa

SEQ ID NO: 11 - RNA, 1410 nt, Artificial Sequence
  Perth/16/2009 (H3N2) NA
augaacccuaaccagaagaucaucacaauuggaagcgugucccugaccauuucgacgauuugcuucuucaugcaaaucgcgaucuugauuaccaccgucacccugcauuucaagcaauacgaauucaacuccccgccaaacaaccaagucaugcucugcgagcccaccaucaucgaacgcaacaucaccgagaucguguaccuuaccaacacuaccaucgaaaaggagauuugccccaaguuggccgaauaccggaacuggagcaagccccagugugacaucacgggauuugcgccauucagcaaggauaacucgaucagacuuuccgccgggggcgacauuugggucacucgggagccuuacgugagcugcgacccggacaagugcuaccaauucgcacucggacaggguaccacccugaacaacguccauagcaacaacaccgugcgcgauagaaccccguaccgcacccuccucaugaacgaacugggagugccguuccacuugggaaccaaacaagucugcauugcaugguccuccuccuccugccacgacggcaaagccuggcuucacguuugcaucaccggcgacgacaagaaugcgacggccuccuucauauacaaugguagacucguggauagcguggugucaugguccaaggaaauucucaggacucaggagucagagugcgugugcaucaacgggacuugcacugucgugaugaccgacggaucggccuccggaaaggccgacacuaagauccucuucaucgaggagggaaagaucgugcacacuucuacccugagcggcucggcucagcaugucgaagagugcucgugcuacccccgguaucccgggguccgcugcgugugccgggacaauuggaaaggcucaaaccgccccaucguggacauuaacaucaaggaccacuccaucgugagcuccuacguaugcagcgggcuggucggggauaccccgcggaagaacgauuccucguccuccucccacugccuggacccuaacaacgaagagggaggccacggagugaagggaugggcuuuugacgauggcaacgacguguggaugggcaggacuauuuccgaaaagucccggcugggauacgaaaccuucaaggucaucgagggcugguccaacccgaagucaaagcuccagaucaaccgccaggucaucguggauaggggcaauagauccggcuacuccgggaucuucagcguggaagggaaguccugcauuaaccgaugcuucuacguggaacucauucggggucggaaggaggaaaccgaagugcuguggacuucgaacucaaucgugguguuuugugggaccuccggaacuuacggaacuggguccuggccugacggugccgacaucaaccuuaugccgaucuaa

SEQ ID NO: 12 - RNA, 1701 nt, Artificial Sequence
  A/Wisconsin/588/2019
augaaagccauccuuguugucaugcuguacacauucaccaccgcaaaugcggauacccuguguaucggcuaccacgcaaauaauuccaccgacaccguugauaccguccuggaaaagaacgugacagugacucacagcgucaaucuccuugaggauaaacauaauggcaagcugugcaagcugagaggcguggcuccccugcaucugggaaagugcaacaucgcugguuggauccucgggaacccagagugugagucccucucaaccgcacggucuuggucauacaucguggagacuagcaauucagacaacggcacaugcuaccccggugacuucauuaacuacgaggagcugagagaacagcugaguuccgugucauccuucgagagauucgaaaucuuccccaaaaccuccuccuggcccaaucaugacuccgacaauggagugacagccgcuuguccccacgccggugccaagaguuucuauaagaaccucaucuggcuggugaaaaagggcaaguccuaucccaaaauuaaccagaccuacauuaacgauaaggggaaagaaguccugguccugugggggauacaccaccccccuaccaucgccgaccagcagucucuguaucagaacgccgacgccuacguguucguggguaccagccguuauaguaaaaaguucaagccagaaauugccaccagaccuaaggugcgcgaccaggagggccgcaugaacuacuacuggacccugguggaaccuggcgacaagauuacauucgaggccacugggaaccugguggcacccagauacgccuuuacaauggaacgggaugcugggagcggaaucauuaucuccgauaccccuguccacgacugcaauacuaccugucagaccccagaaggcgcuaucaauaccucucugccuuuccaaaacgugcacccuaucacuaucgggaaaugucccaaguaugugaaaagcaccaaacugcgccuggcaaccggucugagaaaugugcccuccauccagucccgcggcuuguucggugcaaucgcuggcuuuaucgaggguggcuggacuggaauggucgauggcugguacggcuaccaucaccagaacgagcagggguccggguaugcugccgaccugaaaagcacucagaacgccaucgauaaaaucacuaacaaggugaacuccgugaucgaaaagaugaauacacaguucacagcaguuggcaaggaguucaaccaccuggaaaaacggauagagaaccugaauaagaaagucgaugauggcuuucuggacaucuggacuuacaaugccgagcugcuggugcuccuggaaaacgagcggacacuggauuaucacgacucaaacgugaagaaccuguaugaaaaggugcguaaccagcugaaaaacaacgccaaggaaaucggcaauggcuguuucgaauuuuaccacaagugugauaauaccuguauggagagcguuaagaacgggacuuacgacuacccaaaauacagcgaggaggccaagcugaaccgggagaagaucgacggcgucaaacucgacuccacuagaauauaccagauucucgccaucuauagcacaguggcaucaagucucguccugguggugucacugggagccaucagcuuuuggaugugcagcaauggaucccuccaguguaggaucugcaucuaa

SEQ ID NO: 13 - RNA, 1701 nt, Artificial Sequence
  A/Tasmania/503/2020
augaagaccaucaucgcucuguccuacauccugugccugguguuugcucagaaaauccccgggaaugacaauuccacugccacucucugccugggccaucaugccgugccaaauggaaccauugucaagacuauaacaaaugaccgcaucgaagugaccaacgcuaccgagcugguucagaacagcaguauuggagaaaucugcgauuccccacaccagauacuggauggcggcaacugcacccugaucgacgcacugcugggugacccucagugcgacggauuucagaauaaggagugggaccuuuucguugagcgcagcagagccaauagcaacugcuacccguacgacgugccggauuacgccagucuucgaagccuggucgcauccagcgggacacuggaguuuaagaaugaguccuuuaauuggacaggcgugaagcagaacgggacuagcagcgcaugcauucggggcaguagcucauccuucuuuagccgacugaacuggcugacccaccucaacuacacauaccccgcacugaaugugacuaugccaaacaaagaacaguuugacaaacuguacaucuggggagugcaccauccuagcacagacaaggaccagaucagccuguuugcccagcccagcggcaggauuaccguguccacaaaacggucacagcaagccgugaucccuaauauuggaucccgcccccggauaagggacaucccuagucgcaucaguaucuacuggaccaucgugaagcccggagauaucuugcucaucaauagcacuggcaaccucauugcccccaggggcuauuuuaagaucagaagcggcaaguccagcauuaugcgcagcgacgcacccauuggcaagugcaaguccgagugcaucacuccuaauggguccaucccaaacgacaagccauuccaaaaugucaacagaaucaccuacggggcuugcccccgcuacgugaagcagaguacacugaaacuggccaccgggaugcgcaacgugcccgagaagcaaacuagaggcaucuuuggagcuaucgcuggcuucauugagaauggcugggaggguaugguggacggcugguacggauuccgccaccagaauagcgaaggcagaggccaggcagcagacuugaaguccacccaggccgccauugaucagaucaacggcaaacugaaucggcuuauuggaaaaacaaacgagaaguuccaucagauugagaaggaguuuagcgagguggagggccgcgugcaggaucuggaaaaguacguugaagacaccaagaucgaccuguggucauacaaugcagagcugcucguugcccuggaaaaucagcacacaauugaccuuacagacuccgaaaugaauaagcucuuugaaaagaccaagaagcagcugcgcgagaacgccgaggauauggggaacgguuguuuuaagaucuaccacaagugugacaacgccugcauuggguccauccgaaaugaaacauacgaccacaacguguauagagaugaggcccugaacaaccgauuccagauuaagggagucgagcugaagaguggcuauaaggacuggauccuguggaucucauucgccaugucaugcuuccuucuguguauugcucugcucggcuucaucaugugggcuugccagaaaggcaauauccggugcaacaucugcaucuaa

SEQ ID NO: 14 - RNA, 1749 nt, Artificial Sequence
  B/Washington/02/2019
augaaagcaaucauagugcugcugaugguggugacuagcaaugccgaucggaucugcaccggcaucacuuccaguaacagcccucauguggucaaaaccgccacacagggcgaggugaacgugaccggagugauuccacugacaacuacaccaacgaagagucacuucgccaaccugaagggcaccgaaacacgaggcaagcucugccccaagugucugaauugcaccgaccuggacgucgcuuugggccgcccuaaauguaccggcaaaauaccuuccgccagaguguccauccugcacgaggugcgccccgugaccuccggguguuuucccauaaugcacgaccgcacuaaaauccgccagcugcccaaucuucugaggggguacgaacaugucaggcuguccacucacaacgugaucaacgcagaagacgcccccggaaggccuuaugagauuggaaccaguggguccugcccaaacauuaccaacggcaacggcuucuucgccacuauggccugggccgugccaaagaacaagaccgccaccaacccccugacaauugaagucccuuacaucugcacagagggagaggaucagaucaccguguggggguuucacucugauaacgaaacucagauggccaagcuguacggggauucuaaaccccagaaguucaccaguagcgcuaacggggugaccacccauuaugugucucagaucggagguuucccaaaucagaccgaggacggcggacugccccagucuggaaggaucguaguggacuauauggugcagaagaguggaaaaaccggcaccauuaccuaucagcgcggcauacugcugccacagaagguguggugugcuuccggcagguccaagguuaucaaagggucccucccccugaucggcgaagcagauugucugcacgagaaguacggcggacugaauaagagcaaacccuacuacaccggagaacacgcuaaggcaauugggaauuguccgaucugggugaagacgccccugaaacuggccaauggcacaaaauaccggccccccgcuaagcugcugaaggaacggggguucuucggcgccauagccggcuuucuggagggaggcugggagggcaugauagccggguggcacggcuacacuucccauggggcucacgggguggcuguggccgccgaccugaagucuacgcaggaagcuaucaacaaaaucacuaagaaccugaacagccugucggaauuggaggucaagaaucugcagcggcugagcggcgccauggaugagcugcacaaugagauccuggagcuugacgagaaggucgaugaucuucgggccgauacaauuaguagccaaauugaguuggccgugcugcucagcaacgaaggcauaaucaacagcgaggacgagcaccuccuggcucuggagagaaagcugaagaagaugcucggcccuagcgcaguugagaucggaaacggcugcuucgaaaccaagcacaagugcaaccagaccugccuggacaggaucgcggcaggaacauucgacgcuggggaauucagccuccccaccuucgacagccugaacaucacagccgccagucugaaugaugacggacuggauaaccauaccauccugcuguacuacucuaccgcugcuuccucccuggccgugacauugaugaucgcaaucuuugugguuuauauggugagccgagacaacgucaguugcaguaucugccuuuaa

SEQ ID NO: 15 - RNA, 1755 nt, Artificial Sequence
  B/Phuket/3073/2013
augaaagccaucauugugcugcugaugguuguuacaagcaacgccgaccgcaucugcaccgggauuacaagcagcaauagcccucacguggugaagacagcaacacagggagaggugaacgugaccggcgugauuccacugacaaccaccccaacuaaaucuuacuuugcaaaccugaaagggacacggaccagaggaaagcugugcccugauugccugaauugcacagaccuggacguggcccugggcagaccaaugugcgugggcacuacaccaagcgccaaggccuccauccuccaugaggugcggcccgugacuucuggauguuuccccauuaugcacgacagaaccaagauuagacagcugccaaaccugcuccgcggcuacgagaaaauucgccugucuacacagaaugugaucgacgccgagaaggcuccaggaggaccauacagacuggggacuucuggcagcugcccuaacgccaccucuaagaucggguucuucgcaaccauggcuugggccgugccuaaagacaauuacaagaaugccaccaauccacugacugucgaggugccauauauuugcacagagggggaggaccagaucacuguguggggcuuucauagcgauaauaagacucagaugaagucucucuacggcgacucuaacccucagaaguucaccuccucugccaacggggugacaacacacuacgugucccagaucggggacuuuccugaccagaccgaggauggaggacugccucagucuggacgcaucgugguggacuauaugaugcagaagccugggaagaccggcacuaucguguaccagaggggcgugcugcugccccaaaagguguggugugccuccggaagaagcaaagugauuaagggaucccugccucugauuggggaggccgauugccugcaugaagaguauggagggcugaacaaguccaagccauacuauacaggaaagcacgcaaaagccaucggcaacugucccaucugggucaaaacuccucugaagcuggccaacggcaccaaauaccgcccuccagccaagcugcugaaagaacgcggauucuucggcgccauugcaggguuucuggaaggaggcugggagggcaugauugcuggauggcacggauauaccucucacggcgcucacgggguggccguggccgccgaucugaaguccacacaggaggcaauuaacaagaucaccaagaaucugaauucacuguccgagcucgaagugaaaaaccugcagcgccuguccggcgccauggacgagcugcacaaugaaauccuggagcuggacgagaagguggacgaccugcgggcugacacuaucagcagccagaucgagcuggcagugcugcugagcaaugagggcaucaucaacucagaagacgaacaccuccuggcacuggaaaggaaacucaagaagaugcugggccccuccgcaguggacauugggaacggcuguuucgaaaccaagcauaaguguaaccagacuugucuggauaggaucgcagcaggaaccuucaacgccggcgaauuuucucugccaacauuugacucccugaacaucacagcugcaucccugaacgacgacggacuggacaaucacaccauccugcuguacuacucuacugccgcuagcucccuggccgugacccugaugcuggccaucuucaucguguacaugguuuccagggauaacgugucuuguagcauuugccuguaa

SEQ ID NO: 16-18 - skipped (3 entries)

SEQ ID NO: 19 - RNA, 140 nt, Artificial Sequence
  5UTR
ggacagaucgccuggagacgccauccacgcuguuuugaccuccauagaagacaccgggaccgauccagccuccgcggccgggaacggugcauuggaacgcggauuccccgugccaagagugacucaccguccuugacacg

SEQ ID NO: 20 - RNA, 100 nt, Artificial Sequence
  3UTR sequence
cggguggcaucccugugaccccuccccagugccucuccuggcccuggaaguugccacuccagugcccaccagccuuguccuaauaaaauuaaguugcauc

SEQ ID NO: 21 - skipped

SEQ ID NO: 22 - RNA, 11 nt, Artificial Sequence
  5 UTRs derived from CMV immediate-early 1 (IE1)
gggauccuacc

SEQ ID NO: 23 - RNA, 110 nt, Artificial Sequence
  Poly(A) tail
aaaaaaaaaaaaaaaaaaaaaaaaaaaaaagcauaugacuaaaaaaaaaaaaaaaaaaaaaaaaaaaaaaaaaaaaaaaaaaaaaaaaaaaaaaaaaaaaaaaaaaaaaa

SEQ ID NO: 24 - protein, 566 aa, Artificial Sequence
  CA09 H1 antigen
MKAILVVLLYTFATANADTLCIGYHANNSTDTVDTVLEKNVTVTHSVNLLEDKHNGKLCKLRGVAPLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGSSRYSKKFKPEIAIRPKVRDREGRMNYYWTLVEPGDKITFEATGNLVVPRYAFAMERNAGSGIIISDTPVHDCNTTCQTPKGAINTSLPFQNIHPITIGKCPKYVKSTKLRLATGLRNIPSIQSRGLFGAIAGFIEGGWTGMVDGWYGYHHQNEQGSGYAADLKSTQNAIDEITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDGVKLESTRIYQILAIYSTVASSLVLVVSLGAISFWMCSNGSLQCRICI

SEQ ID NO: 25 - RNA, 1941 nt, Artificial Sequence
  H3 sub type of Influenza virus hemagglutinin
ggacagaucgccuggagacgccauccacgcuguuuugaccuccauagaagacaccgggaccgauccagccuccgcggccgggaacggugcauuggaacgcggauuccccgugccaagagugacucaccguccuugacacgaugaaaaccauaaucgcgcucucauacauacuuugccuggucuuugcccaaaagaucccuggcaacgacaacucaaccgcgacccuuugccucggccaucacgccgugccgaacggcacuaucgucaagaccaucacaaacgaccgcaucgaagugaccaacgcgacugagcuagugcagaacuccagcauuggagagauuugcgauucuccacaccaaauccuggacggagagaauuguaccuugaucgacgcgcugcugggggauccgcagugcgacggauuccagaacaagaaaugggaccuuuucguggaacggagcaaggcauacucgaauugcuaccccuacgaugugcccgacuacgccucgcugcgguccuuggucgcuuccuccgggacccuggaauucaaaaacgagagcuuuaauuggaccggagugacccagaauggcaccucgagcgccugcauucggggcuccuccucgagcuucuucagccgccugaacuggcucacucaccucaacuacaccuacccggcacugaacgugaccaugccgaacaaggaacaauucgacaagcucuacauuuggggggugcaucacccggguaccgauaaggaccagaucuuccucuacgcccaauccucgggccggaucaccguguccacuaagcgcucgcagcaggccgugaucccgaacauuggaagcagaccccgcauucgcgacauuccaucgaggaucucgaucuacuggacgauugucaagccuggcgacauccuccucauuaacuccaccgggaaccucaucgccccucgggguuauuucaagauccgcagcgggaaguccuccaucaugagaagcgaugcccccauuggaaagugcaaguccgaguguaucacaccuaacggaagcauucccaaugacaagccauuccagaacgugaacagaauuaccuacggagcuugcccucgcuacgucaaacauucgacccucaaguuggcgacuggaaugcgcaacgugccggagaagcaaacccgggggaucuucggggcuaucgcgggauucaucgaaaauggaugggaaggaauggucgaugguugguacgguuucagacaccagaacuccgaggggcggggccaggccgcagaccugaaguccacucaggccgcgauugaccagaucaacggaaagcucaacagacucauuggaaagaccaacgaaaaguuccaccaaaucgaaaaggaauucuccgaaguggagggccgggugcaagaccuggagaaguacguggaggacacuaagaucgaccuuuggagcuauaacgcagaacuccuuguggcccuggaaaaccagcacaccaucgaccugaccgauucagagaugaacaagcucuuugagaaaacuaagaagcaacuccgggaaaacgcugaggacaugggaaauggaugcuuuaagaucuaccacaagugcgacaacgccugcauugaguccauacggaacgaaacuuacgaccauaacgucuaccgggaugaagcccugaacaacagauuccagaucaagggcguggagcugaaguccggcuacaaagauuggauccuguggauuuccuucgcgauuucaugcuucuugcucugcguggcccuccugggauucauaaugugggccugucagaagggcaacauuaggugcaacauaugcauauaacggguggcaucccugugaccccuccccagugccucuccuggcccuggaaguugccacuccagugcccaccagccuuguccuaauaaaauuaaguugcauc